(19) United States
(12) Patent Application Publication
(10) Pub. No.: US 2026/0109985 A1
(43) Pub. Date: Apr. 23, 2026

(54) THERAPY BY TRANS-SPLICING OF OPA1 PRE-MESSENGER RNAS FOR THE TREATMENT OF DISEASES ASSOCIATED WITH OPA1 GENE MUTATIONS

(71) Applicants: Centre National de la Recherche Scientifique, Paris (FR); Institut National de la Santé et de la Recherche Médicale, Paris (FR); Universite d'Angers, Angers (FR)

(72) Inventors: Yannick LE DANTEC, Angers (FR); Guy LENAERS, Avrille (FR); Olivier BARIS, Avrille (FR); Arnaud CHEVROLLIER, Savennieres (FR); Salim KHIATI, Avrille (FR)

(73) Assignees: Centre National de la Recherche Scientifique, Paris (FR); Institut National de la Santé et de la Recherche Médicale, Paris (FR); Universite d'Angers, Angers (FR)

(21) Appl. No.: 19/116,221

(22) PCT Filed: Sep. 29, 2023
(86) PCT No.: PCT/EP2023/077001

§ 371 Date: Mar. 27, 2025

(30) Foreign Application Priority Data
Sep. 30, 2022 (EP) 22199015.3

(51) Int. Cl. C12N 15/113; C12N 15/86

(52) U.S. Cl. CPC C12N 15/1137; C12N 15/86; C12Y 306/05005; C12N 2320/33; C12N 2750/14143

(57) ABSTRACT

The present invention provides pre-mRNA trans-splicing molecules (RTMs) that are useful for correcting mutations in the OPA1 gene. Also provided are methods of using the RTMs as gene therapy (e.g., ex vivo and in vivo gene therapy) for the treatment or prevention of diseases or disorders associated with OPA1 mutations.

### RELATED APPLICATION

The present application claims priority to European Patent Application No. EP 22 199 015 filed on Sep. 30, 2023, which is incorporated herein by reference in its entirety.

### BACKGROUND OF THE INVENTION

Hereditary Optic Neuropathies (HONs) are a genetically diverse group of disorders mainly characterized by visual loss and optic atrophy. Several genetic defects altering primary mitochondrial functions have been proposed to contribute to the development of syndromic and non-syndromic optic neuropathies. A subset of HONs, including Autosomal Dominant Optic Atrophy (ADOA), Autosomal Dominant Optic Atrophy plus (ADOA+ or ADOAplus) and Behr's Syndrome, is caused by mutations in the OPA1 gene. HONs collectively affect nearly 400,000 people worldwide, with 37,000 in Europe and 3,000 in France (Lenaers et al., Progress in Retinal and Eye Research, 2021, 83:100935; Kjer et al., Acta Ophthalmol Scand., 1996, 74 (1): 3-7).

ADOA is believed to be the most common HON with a disease prevalence in the range of 1:10,000 to 1:50,000 (Kjer et al., Acta Ophthalmol. Scand., 1996, 4 (1): 3-7). ADOA is characterized by bilateral loss of vision in the central visual field and impaired color perception due to progressive degeneration of the retinal ganglion cells (RGCs) and their axons forming the optic nerve. Because of this degeneration, visual information is no longer transmitted to the higher visual areas of the brain. ADOA onset generally occurs in the first decade of life, and is usually diagnosed before adulthood, but signs have been reported as early as 1 year of age. It is primarily a pediatric disease and those affected will typically show signs of nerve deterioration slowing by the age of 15. ADOA+is a syndromic ADOA and accounts for approximately 20% of all ADOA cases. Symptoms of ADOA+presentation typically start to occur within the first decade of life and are characterized by bilateral and symmetric progressive visual loss. Sensorineural deafness along with other extra-ocular manifestations may appear, such as chronic progressive external ophthalmoplegia, proximal myopathy, ataxia and axonal sensory motor polyneuropathy, beginning in the second and third decades of life. Behr's syndrome, related to bi-allelic OPA1 variants, is characterized by the association of early onset optic atrophy with spinocerebellar degeneration resulting in ataxia, pyramidal signs, peripheral neuropathy and developmental delay.

There is currently no established medical treatment for OPA1-related inherited optic neuropathies. Coenzyme Q-10 (CoQ), Idebenone, and nutritional supplements, such as vitamin B12, vitamin C and lutein, have been suggested to reduce reactive oxygen species-induced stress in the optic nerve (Carelli et al., Curr. Opin. Neurol., 2013, 26 (1): 52-58). Topical agents deemed to be neuroprotective or antiapoptotic, such as brimonidine, have also been recommended, although evidence of their efficacy remains anecdotal (Carelli et al., Curr. Opin. Neurol., 2013, 26 (1): 52-58). Glasses or contact lenses, which may correct coexisting farsightedness, nearsightedness and astigmatism, will not repair or correct the vision loss caused by ADOA. ADOA patients may find improvement in visual functioning with low-vision aids (e.g., magnifiers, large-print devices, talking watches, tinted lenses, electronic magnifiers, or other adaptive devices).

AODA, AODA+ and Behr's Syndrome are commonly associated with mutations in the nuclear OPA1 gene. OPA1 encodes a mitochondrial dynamin-related GTPase that is involved in mitochondrial membrane dynamics and structural organization (Newman et al., Am. J. Ophthal. 2005, 140 (3): 517-523; Kline et al., Arch. Ophthalmol., 1979, 97 (9): 245-251; Delettre et al., Mol. Genet. Metab., 2002, 75 (2): 97-107). Abnormal mitochondrial metabolism and impaired oxidative phosphorylation lead to increases in reactive oxygen species levels and retinal ganglion cells (RGCs) apoptosis (Chun et al., Semin. Pediatr. Neurol., 2017, 24 (2): 129-134; Chun et al., Curr. Opin. Ophthalmol. 2016, 27 (6): 475-480). RGCs degeneration, predominantly in the papillomacular bundle, has been suggested as underlying the visual defect (Delettre et al., Mol. Genet. Metab., 2002, 75 (2): 97-107).

The discovery of OPA1 mutations as causative for ADOA was pivotal to deepen the understanding of its pathogenesis, to possibly establish effective therapies, in particular strategies based on gene therapy approaches. Thus, adeno-associated virus (AAV) delivered OPA1 isoform 1 showed significant protection of RGCs in a heterozygous mouse model of pathogenic OPA1 mutation (Opa1del^{TTAG/+}) but did not result in a significant increase in visual acuity (Sarzi et al., Sci Rep., 2018, 8:2468). Overexpression of OPA1 isoform 1 or 7 was found to correct mitochondrial dysfunctions and partially restore visual perception and integration (Maloney et al., Front. Neurosci., 2020, 14:571479). However, this study also confirmed that overexpression of OPA1 isoforms is more deleterious than the pathological condition because it induces an alteration of the mitochondrial network.

It has been reported (Amati-Bonneau et al., Brain, 2008, 131:338-351; Hudson et al., Brain 2008, 131:329-337) that OPA1 mutations can also be associated with other neuropathies such as for example sensorineural hearing loss or sensorimotor neuropathy, or with ataxia, progressive external ophthalmoplegia and mitochondrial myopathy.

Although new therapeutic approaches are being developed and tested, no disease-modifying treatments of hereditary diseases, especially neuropathies, in particular optic neuropathies associated with mutations in the OPA1 gene are yet available. Therefore, there is still a need in the art for novel strategies, in particular strategies allowing a fine regulation of the expression of all the eight OPA1 protein isoforms, which is essential to obtain a therapeutic benefit.

### SUMMARY OF THE INVENTION

The present invention consists in the creation of a versatile gene therapy approach using Spliceosome-Mediated RNA Trans-splicing (SMART™) for the treatment of diseases associated with mutations of the OPA1 gene. In particular, the present Inventors have identified specific target intronic and exonic sequences on the mutated endogenous OPA1 pre-messenger RNA (pre-mRNA), which are located downstream of the alternatively spliced exons and upstream of the main exons carrying mutations. Therapeutic molecules, such as pre-mRNA trans-splicing molecules (RTMs or PTMs), have been designed based on the specific target intronic sequences, that function to compensate the defective OPA1 gene by trans-splicing of the OPA1 pre-mRNAs, in order to eliminate the mutations of the mature RNAs of OPA1. The present invention allows to maintain the endogenous regulation of gene expression as well as the respective abundance of the eight isoforms of OPA1. It makes possible the correction of transcripts resulting from dominant negative and haploinsufficient alleles. A pre-mRNA trans-splicing molecule according to the invention can correct nearly 90% of all known pathogenic mutations within the human OPA1 gene. In addition, a pre-mRNA trans-splicing molecule may be used to treat not only non-syndromic patients suffering from blindness but also syndromic patients who also experience sensorineural deafness and polyneuropathy. Indeed, only the vectorization of the pre-mRNA trans-splicing molecule needs to be adapted to the transduction of the target cells (either retinal ganglion cells or cells of the auditory nerve or cells of the Central Nervous System (CNS) or Peripheral Nervous System (PNS)), the pre-mRNA trans-splicing molecule remains unchanged.

Consequently, the present invention provides a nucleic acid OPA1 pre-mRNA trans-splicing molecule comprising operatively linked in a 5′-to-3′ direction or a 3′-to-5′ direction:
(a) a binding domain configured to bind to an OPA1 target sequence comprising an intron, an intron/exon junction or an exon;
(b) a splicing domain configured to mediate trans-splicing; and
(c) a coding domain comprising at least one functional OPA1 exon, wherein the nucleic acid OPA1 pre-mRNA trans-splicing molecule is configured to trans-splice the endogenous mutated OPA1 pre-mRNA adjacent to the OPA1 target sequence, thereby replacing the endogenous mutated OPA1 exon with the functional OPA1 exon and correcting a mutation in OPA1 transcripts.

In certain embodiments, the OPA1 target sequence comprising an intron, an intron/exon junction or an exon is Homo sapiens OPA1 intron 8-9/Exon 9.

In certain embodiments, the OPA1 target sequence comprising an intron, an intron/exon junction or an exon is-Homo sapiens OPA1 intron 8-9/Exon 9 consisting of SEQ ID NO: 1.

In certain embodiments, the binding domain binds to the OPA1 target sequence at a binding site within or encompassing nucleotides 1 to 52 of SEQ ID NO: 1, or a binding site within or encompassing nucleotides 1 to 50 of SEQ ID NO: 1, or at a binding site within or encompassing nucleotides 261 to 315 of SEQ ID NO: 1, or at a binding site within or encompassing nucleotides 361 to 603 of SEQ ID NO: 1, or at a binding site within or encompassing nucleotides 361 to 460 of SEQ ID NO: 1, or at a binding site within or encompassing nucleotides 375 to 460 of SEQ ID NO: 1; or nucleotides 461 to 603 of SEQ ID NO: 1, or at a binding site within or encompassing nucleotides 510 to 784 of SEQ ID NO: 1, or at a binding site within or encompassing nucleotides 510 to 756 of SEQ ID NO 1, or at a binding site within or encompassing nucleotides 510 to 726 of SEQ ID NO 1, or at a binding site within or encompassing nucleotides 518 to 784 of SEQ ID NO 1, or at a binding site within or encompassing nucleotides 518 to 756 of SEQ ID NO 1, or at a binding site within or encompassing nucleotides 518 to 726 of SEQ ID NO 1, or at a binding site within or encompassing nucleotides 561 to 784 of SEQ ID NO 1, or at a binding site within or encompassing nucleotides 561 to 756 of SEQ ID NO 1, or at a binding site within or encompassing nucleotides 561 to 726 of SEQ ID NO 1, or at a binding site within or encompassing nucleotides 627 to 784 of SEQ ID NO 1, or at a binding site within or encompassing nucleotides 627 to 756 of SEQ ID NO 1, or at a binding site within or encompassing nucleotides 627 to 726 of SEQ ID NO 1, or at a combination of said binding sites.

In certain embodiments, the binding domain is complementary to six or more consecutive nucleotides of a binding site within or encompassing nucleotides 1 to 52 of SEQ ID NO: 1, or nucleotides 1 to 50 of SEQ ID NO: 1, or nucleotides 261 to 315 of SEQ ID NO: 1, or nucleotides 361 to 603 of SEQ ID NO: 1, or nucleotides 361 to 460 of SEQ ID NO: 1, or nucleotides 375 to 460 of SEQ ID NO: 1; or nucleotides 461 to 603 of SEQ ID NO: 1, or nucleotides 510 to 784 of SEQ ID NO: 1, or nucleotides 510 to 756 of SEQ ID NO 1, or nucleotides 510 to 726 of SEQ ID NO 1, or nucleotides 518 to 784 of SEQ ID NO 1, or nucleotides 518 to 756 of SEQ ID NO 1, or nucleotides 518 to 726 of SEQ ID NO 1, or nucleotides 561 to 784 of SEQ ID NO 1, or nucleotides 561 to 756 of SEQ ID NO 1, or nucleotides 561 to 726 of SEQ ID NO 1, or nucleotides 627 to 784 of SEQ ID NO 1, or nucleotides 627 to 756 of SEQ ID NO 1, or nucleotides 627 to 726 of SEQ ID NO 1, or at a combination of said binding sites.

In certain embodiments, the binding domain comprises, or consists of SEQ ID NO: 2 (BD #114) or is a fragment thereof; or the binding domain comprises, or consists of, SEQ ID NO: 3 (BD #128) or is a fragment thereof; or the binding domain comprises, or consists of, SEQ ID NO: 4 (BD #162) or is a fragment thereof; or the binding domain comprises, or consists of, SEQ ID NO: 5 (BD #135) or is a fragment thereof; or the binding domain comprises, or consists of, SEQ ID NO: 6 (BD #106) or is a fragment thereof; or the binding domain comprises, or consists of, SEQ ID NO: 7 (BD #100) or is a fragment thereof; or the binding domain comprises, or consists of, SEQ ID NO: 8 (BD #130) or is a fragment thereof; or the binding domain comprises, or consists of, SEQ ID NO: 9 (BD #158) or is a fragment thereof; or the binding domain comprises, or consists of, SEQ ID NO: 10 (BD WO 2024/068898-6-PCT/EP2023/077001 #166) or is a fragment thereof; or the binding domain comprises, or consists of, SEQ ID NO: 11 (BD #196) or is a fragment thereof; or the binding domain comprises, or consists of, SEQ ID NO: 12 (BD #224) or is a fragment thereof; or the binding domain comprises, or consists of, SEQ ID NO: 13 (BD #209) or is a fragment thereof; or the binding domain comprises, or consists of, SEQ ID NO: 14 (BD #239) or is a fragment thereof; or the binding domain comprises, or consists of, SEQ ID NO: 15 (BD #267) or is a fragment thereof; or the binding domain comprises, or consists of, SEQ ID NO: 16 (BD #217) or is a fragment thereof; or the binding domain comprises, or consists of, SEQ ID NO: 17 (BD #247) or is a fragment thereof; or the binding domain comprises, or consists of, SEQ ID NO: 18 (BD #275) or is a fragment thereof; or the binding domain is a combination of said binding domains.

In certain embodiments, the mutation is in any one of OPA1 exons 9 to 31.

In certain embodiments, the mutation is associated with a hereditary optic neuropathy, in particular Autosomal Dominant Optic Atrophy (ADOA), Autosomal Dominant Optic Atrophy Plus (ADOA+) and Behr's Syndrome, or an extra-ophthalmic disease, in particular sensorineural deafness, peripheral neuropathies, Parkinson syndrome, mitochondrial myopathy, and heart disease.

In certain embodiments, the mutation is associated with a hereditary optic neuropathy selected from Autosomal Dominant Optic Atrophy (ADOA), Autosomal Dominant Optic Atrophy Plus (ADOA+) and Behr's Syndrome.

In certain embodiments, the mutation is associated with the optic neuropathy: susceptibility to normal tension glaucoma.

In certain embodiments, the mutation is associated with an extra-ophthalmic disease selected from sensorineural deafness, peripheral neuropathies, Parkinson syndrome and mitochondrial myopathy.

In certain embodiments, the mitochondrial myopathy is mitochondrial depletion syndrome 14.

In certain embodiments, the coding domain comprises functional OPA1 exons 9 to 31.

In certain embodiments, the nucleic acid OPA1 pre-mRNA trans-splicing molecule consists of SEQ ID NO: 27 (OPA1 RTMₜₕ#114), or SEQ ID NO: 28 (OPA1 RTMₜₕ#128), or SEQ ID NO: 30 (OPA1 RTMₜₕ#162), or SEQ ID NO: 29 (OPA1 RTMₜₕ#135) or SEQ ID NO: 26 (OPA1 RTMₜₕ#106), or SEQ ID NO: 31 (OPA1 RTMₜₕ#100), or SEQ ID NO: 32 (OPA1 RTMₜₕ#130), or SEQ ID NO: 33 (OPA1 RTMₜₕ#158), or SEQ ID NO: 34 (OPA1 RTMₜₕ#166), or SEQ ID NO: 35 (OPA1 RTMₜₕ#196), or SEQ ID NO: 36 (OPA1 RTMₜₕ#224), or SEQ ID NO: 37 (OPA1 RTMₜₕ#209), or SEQ ID NO: 38 (OPA1 RTMₜₕ#239), or SEQ ID NO: 39 (OPA1 RTMₜₕ#267), or SEQ ID NO: 40 (OPA1 RTMₜₕ#217), or SEQ ID NO: 41 (OPA1 RTMₜₕ#247), or SEQ ID NO: 42 (OPA1 RTMₜₕ#275).

The present invention further provides a cloning vector recombinant adeno-associated virus comprising a nucleic acid OPA1 trans-splicing molecule as described above.

In certain embodiments, the cloning vector is selected from plasmids, minicircles, cosmids, YAC vectors, BAC vectors, and viral vectors. The viral vector may be an Adeno-Associated Virus (AAV), in particular a recombinant Adeno-Associated Virus (rAAV), a single-stranded Adeno-Associated Virus (ssAAV) or a self-complementary Adeno-Associated Virus (scAAV).

In certain embodiments, the recombinant adeno-associated virus targets retinal ganglion cells, photoreceptor cells, amacrine cells, horizontal cells, bipolar cells, or cells of the auditory nerve.

The present invention further provides a cell comprising a nucleic acid OPA1 trans-splicing molecule as described above or a cloning vector as described above.

The present invention also provides a pharmaceutical composition comprising a nucleic acid OPA1 trans-splicing molecule as described above, or a cloning vector as described above, or a cell as described above, and at least one pharmaceutically acceptable carrier or excipient.

The present invention further provides a nucleic acid OPA1 trans-splicing molecule as described above, or a cloning vector as described above, or a cell as described above, or a pharmaceutical composition as described above, for use in the treatment or prevention of a disease or disorder associated with a OPA1 gene mutation in subject.

In certain embodiments, the disease or disorder associated with a OPA1 gene mutation is a hereditary optic neuropathy, in particular Autosomal Dominant Optic Atrophy (ADOA), Autosomal Dominant Optic Atrophy Plus (ADOA+) and Behr's Syndrome, or an extra-ophthalmic disease, in particular sensorineural deafness, peripheral neuropathies, Parkinson syndrome, mitochondrial myopathy, and heart disease.

In certain embodiments, the disease or disorder associated with a OPA1 gene mutation is a hereditary optic neuropathy selected from Autosomal Dominant Optic Atrophy (ADOA), Autosomal Dominant Optic Atrophy Plus (ADOA+) and Behr's Syndrome.

In certain embodiments, the disease or disorder associated with OPA1 gene mutation is the optic neuropathy: susceptibility to normal tension glaucoma.

In certain embodiments, the disease or disorder associated with a OPA1 gene mutation is an extra-ophthalmic disease selected from sensorineural deafness, peripheral neuropathies, Parkinson syndrome and mitochondrial myopathy.

In certain embodiments, the mitochondrial myopathy is mitochondrial depletion syndrome 14.

These and other objects, advantages and features of the present invention will become apparent to those of ordinary skill in the art having read the following detailed description of the preferred embodiments.

### BRIEF DESCRIPTION OF THE DRAWING

FIG. 1. Schematization of endogenous OPA1 pre-mRNA cis-splicing regulation and OPA1 pre-mRNA trans-splicing therapeutic approach. In the pathogenic context, alternate splicing of endogenous OPA1 pre-mRNA generates eight pathogenic OPA1 transcripts to ADOA/ADOA+/Behr syndrome either by haploinsufficiency or negative dominance of OPA1 protein isoforms. In the therapeutic context, the OPA1 trans-splicing approach allows the correction of the eight pathogenic OPA1 transcripts by OPA1-specific pre-mRNA trans-splicing molecule (OPA1-RTM or OPA1-PTM) containing antisense and complementary OPA1 binding domain, intronic splicing enhancer and corrective exons. The trans-splicing reaction, mediated by the endogenous spliceosome ribonucleoprotein complex, leads to the pathogenic exons replacement by the exogenous corrective exons, generating eight trans-spliced (chimeric) mRNAs and the correction of the haploinsufficiency or negative dominance of the OPA1 protein isoforms.

FIG. 2. Homo sapiens intron8-9/EXON9 OPA1-minigene (6606 bp). Restriction map of the Homo sapiens intron8-9/EXON9 OPA1-minigene plasmid used in a minigene assay for the identification of efficient trans-splicing binding domains. The intron8-9/EXON9 sequence of the OPA1 Homo sapiens gene was cloned between the EcoRV and NotI restriction sites through the Gibson cloning assembly method.

FIG. 3. Agarose gel electrophoresis of Homo sapiens OPA1 intron8-9/EXON 9 antisense binding domains banks edition via CviJI* endonuclease digest (A) or sonication (B).

FIG. 4. Restriction map of the RTM0 plasmid used in the minigene assay for the identification of efficient trans-splicing binding domains. The RTM0 plasmid contains a 27-bp spacer, a 8-bp branch point (BP), a 16-pb polypyrimidine Tract (PPT), a 3′-Acceptor Splice Site (3′ASS), the 3′ half part of the AcGFP1 encoding sequence (384-bp), an Internal Ribosome Entry Site (IRES), a 678-bp DsRed monomer sequence preceded by a Kozak sequence.

FIG. 5. Intronic Splicing Enhancer (ISE) cassette into which Homo sapiens OPA1 antisense binding domains were linked. The ISE cassette contains a 27-bp spacer, an 8-bp branch point (BP), a 16-bp PolyPyrimidine Tract (PPT), and a 3′-Acceptor Splice Site (3′ASS).

FIG. 6. Homo sapiens OPA1-minigene GFP reconstitution assay. (A) Homo sapiens OPA1-minigene trans-splicing principle. Homo sapiens OPA1-minigene encodes a pre-mRNA containing the 5′ half part of the AcGFP1 encoding sequence (336 bp) followed by the Homo sapiens OPA1 intron 8-9/EXON9 (193,637,282-193,638,065) considered as the target. Co-expression of OPA1-specific RTM containing Homo sapiens OPA1 antisense binding domain leads to the hybridization of both pre-mRNAs and competition between OPA1-minigene cis-splicing and trans-splicing with the RTM. Efficient trans-splicing induces the reconstitution of the complete open reading frame of the sequence encoding the fluorescent protein AcGFP1. Trans-splicing events are visualized with fluorescence microscopy (B) showing co-localization between RTM-expressing cells (DsRed+ cells) and trans-splicing-cells (AcGFP1+ cells). OPA1-targeting RTMs inducing the highest GFP+ cells were selected by fluorescence-activated cell sorting (FACS) (C-D) and confirmed by (E) anti-GFP western blotting and (F) RT-PCR analysis.

FIG. 7. Restriction map of the pEF1a-IRES-AcGFP1 bicistronic mammalian expression vector used for therapeutic OPA1 trans-splicing molecule expression.

FIG. 8. Therapeutic effects of OPA1 pre-mRNA trans-splicing in in vitro experiments on DOA-related patients' fibroblasts with bi-allelic OPA1 variants. OPA1 pre-mRNA trans-splicing restored physiological levels of OPA1 isoforms in Western blot (A) and mitochondrial network fusion in comparison to control cell line, without negative impact on the mitochondrial network of control fibroblasts devoid of OPA1 pathogenic variants (B-C).

### DEFINITIONS

Throughout the specification, several terms are employed that are defined in the following paragraphs.

As used herein, the term “subject” refers to a human or another mammal (e.g., primate, mouse, rat, rabbit, dog, cat, horse, pig, livestock, and the like), including laboratory animals, that may or may not have a disease associated with a mutation in the OPA1 gene. Non-human subjects may be transgenic or otherwise modified animals. In many embodiments of the present invention, the subject is a human being. In such embodiments, the subject is often referred to as an “individual” or a “patient”. The terms “individual” and “patient” do not denote a particular age. The term “patient” or “affected subject” more specifically refers to an individual suffering from a disease or condition associated with mutation(s) in the OPA1 gene (i.e., to an individual showing clinical signs of the disease or disorder), or to an individual at risk of developing such a disease or disorder. A subject can be characterized as “at risk” of developing a disease by identifying a mutation in the OPA1 gene associated with the disease. In some embodiments, the subject who is at risk of developing a disease has one or more OPA1 mutations associated with the disease. Additionally, or alternatively, a subject can be characterized as “at risk” of developing a disease if the subject has a family history of the disease.

The term “mutation”, as used herein, refers to any aberrant nucleic acid sequence that causes a defective protein product (e.g., a non-functional protein product, a protein product having reduced function, a protein product having aberrant function, and/or a protein product that is produced in less than normal or greater than normal quantities). Mutations include base pair mutations (e.g., single nucleotide polymorphisms), missense mutations, frameshift mutations, deletions, insertions, splice mutations, and cryptic mutations. In some embodiments, a mutation refers to a nucleic acid sequence that is different in one or more portions of its sequence than a corresponding wild-type nucleic acid sequence or functional variant thereof. In some embodiments, a mutation refers to a nucleic acid sequence that encodes a protein having an amino acid sequence that is different than the sequence of the wild-type protein or functional variant thereof. A “mutated exon” refers to an exon containing a mutation or an exon sequence that reflects a mutation in a different region, such as a cryptic exon resulting from a mutation in an intron. A “mutated intron” refers to an intron containing a mutation or a deletion or an insertion or an inversion that affects the sequence or the level of expression of the gene of interest.

As used herein, the terms “disease or disorder associated with a mutation” or “mutation associated with a disease or disorder” refer to a correlation between a disease or disorder and a mutation. In some embodiments, a disease or disorder associated with a mutation is known or suspected to be wholly or partially, or directly or indirectly, caused by the mutation. For example, a subject having the mutation may be at risk of developing the disease or disorder, and the risk may additionally depend on other factors, such as other (e.g., independent) mutations (e.g., in the same or a different gene), or environmental factors.

The term “OPA1” refers to the human nuclear gene that encodes a mitochondrial protein with similarity to dynamin-related GTPases. The encoded protein localizes to the inner mitochondrial membrane and helps to regulate mitochondrial membrane dynamics and structures. More specifically, the term “OPA1” refers to the human OPA1 gene that is located on chromosome 3q28-929, and more precisely from base pair 193,593,144 to base pair 193,697,811 forward strand on chromosome 3. Mutations in this gene have been associated with neuro-pathologies, more particularly ocular pathologies and/or hearing pathologies. As used herein, the term “disease or disorder associated with mutation(s) in the OPA1 gene” refers to any disease or disorder that is known or suspected to be wholly or partially, or directly or indirectly, caused by a mutation in the human OPA1 gene. Examples of such diseases include Autosomal Dominant Optic Atrophy (ADOA), Autosomal Dominant Optic Atrophy plus (ADOA+) and Behr's Syndrome.

The term “treatment” is used herein to characterize a method or process that is aimed at (1) delaying or preventing the onset of a disease or condition (herein a disease or disorder associated with a mutation in the OPA1 gene); (2) slowing down or stopping the progression, aggravation, or deterioration of at least one symptom of the disease or condition; (3) bringing about amelioration of at least one symptom of the disease or condition; or (4) curing the disease or condition. A treatment may be administered prior to the onset of the disease or condition, for a prophylactic or preventive action. Alternatively, or additionally, a treatment may be administered after initiation of the disease or condition, for a therapeutic action. As used herein, the term “prevention” of a disorder is defined as reducing the risk of onset of a disease, e.g., as a prophylactic therapy for a subject who is at risk of developing a disorder associated with a mutation on the OPA1 gene.

A “pharmaceutical composition” is defined herein as comprising an effective amount of at least one nucleic acid OPA1 trans-splicing molecule according to the invention, and at least one pharmaceutically acceptable carrier or excipient.

As used herein, the term “effective amount” refers to any amount of a compound (e.g., a nucleic acid OPA1 trans-splicing molecule), or composition that is sufficient to fulfil its intended purpose(s), e.g., a desired biological or medicinal response in a cell, tissue, system or subject. An effective amount of a nucleic acid OPA1 trans-splicing molecule is an amount that can elicit a measurable amount of a desirable outcome, e.g., in a method of treatment, an effective amount is an amount that can reduce or ameliorate by a measurable amount, a symptom of the disease or condition that is being treated.

As used herein, the term “pharmaceutically acceptable carrier or excipient” refers to a carrier medium which does not interfere with the effectiveness of the biological activity of the active ingredient(s) and which is not excessively toxic to the host at the concentration at which it is administered. The term includes solvents, dispersion media, antibacterial and antifungal agents, isotonic agents, and the like. The use of such media and agents for pharmaceutically active substances is well known in the art (see for example “Remington's Pharmaceutical Sciences”, E. W. Martin, 18th Ed., 1990, Mack Publishing Co.: Easton, PA, which is incorporated herein by reference in its entirety). In specific embodiments, the term “pharmaceutically acceptable” means approved by a regulatory agency or listed in the U.S./E.U. Pharmacopeia or other generally recognized pharmacopeia for use in mammals, and more particularly in humans.

The terms “approximately” and “about”, as used herein in reference to a number, generally include numbers that fall within a range of 10% in either direction of the number (greater than or less than the number) unless otherwise stated or otherwise evident from the context (e.g., where such number would exceed 100% of a possible value).

### DETAILED DESCRIPTION OF CERTAIN PREFERRED EMBODIMENTS

As mentioned above, the present invention provides nucleic acid trans-splicing molecules (e.g., pre-mRNA trans-splicing molecules (RTMs also called PTMs)) that are useful for correcting mutations in the OPA1 gene. Also provided are methods of using the nucleic acid trans-splicing molecules as gene therapy (e.g., ex vivo and in vivo gene therapy) for the treatment or prevention of diseases or disorders associated with OPA1 mutations, in particular ADOA, ADOA+ and Behr's Syndrome.

### I-Nucleic Acid OPA1 Trans-Splicing Molecules

The present invention is based on the use of spliceosome-mediated RNA trans-splicing (SMaRT™), a technology with the ability to reprogram mRNAs and the proteins they encode. SMART™ is here employed to correct mutations in the human OPA1 gene.

### 1. OPA1 Gene Mutations

The human OPA1 gene is composed of 30 coding exons (exons 1 to 28, exon 4b, exon 5b) distributed across more than 100 kb of genomic DNA on chromosome 3q28-q29. Alternative splicing of exons 4, 4b, and 5b leads to eight mRNA variants, encoding eight isoforms of 924 to 1015 amino acids (Delettre et al., Hum. Genet., 2001, 109 (6): 584-591), the exact balance of which is essential for proper OPA1 functions. The OPA1 proteins are classified as large GTPases of the dynamin-like family, which are imported into the mitochondria by their amino-terminal import-sequence, mitochondrial targeting sequence (MTS), and which are necessary for mitochondrial inner and outer membrane fusions.

As of April 2021, the Human Gene Mutation Database (HGMD) (Stenson et al., Hum. Genet., 2017; 136:665-677; Weisschuch et al., PLOS One, 2021, 16 (7): e0253987) lists more than 400 disease-causing variants in OPA1 while the Leiden Open Variation Database for OPA1 (website: www.lovd.nl/OPA1) (Le Roux et al., Orphanet J. Rare Dis., 2019, 14:214), which entries overlap largely but not completely with HGMD, lists 593 unique public variants. Dominant-negative and haploinsufficiency effects have been proposed as the predominant pathomechanisms for OPA1 variants (Pesch et al., Hum. Mol. Genet., 2001, 10:1359-1368; Le Roux et al., Orphanet J. Rare Dis., 2019, 14:214). Accordingly, the majority of disease-causing variants are predicted to give rise to OPA1 missense amino acid changes, or truncated polypeptides, either due to nonsense, frameshift or splice variants, the latter including deep intronic variants causing the inclusion of pseudoexons into the transcript, or a partial gene deletions or duplications (Bonifert et al., Brain, 2014, 137:2164-2177; Bonifert et al., Mol. Ther. Nucleic Acids, 2016, 5: e390). In addition, structural variants such as copy number variants (CNVs) and inversions are part of the mutation spectrum of OPA1 (Furhmann et al., J Med Genet., 2009; 46:136-44; Almind et al., BMC Med Genet. 2011; 12:49; Weisschuh et al., BMC Med Genet. 2020; 21:236). More than 95% of disease-causing variants are located downstream of the alternative exons in the OPA1 gene.

### 2. Spliceosome-Mediated RNA Trans-Splicing (SMART™)

Splicing is a naturally occurring process that takes place during the generation of fully active messenger RNA (mRNA) and is mediated by the cell's own spliceosome. Within a cell, a pre-mRNA intermediate exists that includes non-coding nucleic acid sequences (introns) and coding nucleic acid sequences (exons) that encode the amino acids forming the gene product. In the pre-mRNA, the introns are interspersed between the exons; they are ultimately excised from the pre-mRNA and the exons are fused together to form the mature RNA (mRNA). The predominant splicing form in eukaryotic cells is cis-splicing, where the exons from one pre-mRNA transcript are joint together. In contrast, RNA trans-splicing involves the joining of exons originating from more than one pre-mRNA transcript. Trans-spliced RNA can encode new proteins or non-coding regulatory transcripts, not only resulting in increased proteome complexity, but also contributing to the regulation of gene expression.

Spliceosome-mediated RNA trans-splicing (SMART™), which utilizes the endogenous cellular splicing machinery to repair inherited genetic defects or mistakes at the pre-mRNA level by replacing mutant exon or exons by their wild-type counterparts, has emerged as a novel technology for the correction of monogenic disorders. It employs an engineered pre-mRNA trans-splicing molecule (RTM) that binds specifically to target pre-mRNA in the nucleus and triggers trans-splicing in a process mediated by the spliceosome. SMART™ can be used to replace 5′, 3′, or internal sequences in an exon-wise manner. This methodology is described in, for example, Puttaraju et al., Nature Biotechnol. 1999, 17:246-252; Gruber et al., Mol. Oncol., 2013, 7 (6): 1056; Avale, Hum. Mol. Genet., 2013, 22 (13): 2603-2611; Rindt et al., Cell Mol. Life Sci. 2012, 69 (24): 4191; Liemberger et al., Int. J. Mol. Sci., 2018, 19:762; US Patent Application Publication Nos. 2006/0246422, 2013/0059901, and U.S. Pat. Nos. 6,083,702; 6,013,487; 6,280,978; 7,399,753; 7,968,334 8,053,232; 8,735,366; 9,303,078 and 9,655,979, which are incorporated herein by reference. See also Berger et al., Mol. Ther., 2015, 23 (5): 918-930; Koller et al., Nucleic Acids Res., 2011, 39 (16): e10⁸; Koller et al., Int. J. Mol. Sci., 2015, 16 (1): 1179-1191; Murauer et al., Hum. Gene Ther. Methods, 2013, 24 (1): 19-27; Rindt et al., Front. Neurosci., 2017, 11:544; Tockner et al., Gene Ther., 2016, 23 (11): 775-784.

The replacement of exonic portions provides several advantages relative to other gene therapeutic approaches, such as complementary DNA (cDNA) therapy. First, for very large genes, the sequence to be replaced can be reduced to a size that facilitates viral packaging and transduction. Second, the increase of wild-type alleles and the concomitant decrease of mutated alleles leads to an enhanced shift in their quantitative proportions, making this technology suitable for the correction of dominant mutations. Third, as trans-splicing takes place at the pre-messenger RNA (pre-mRNA) level, target gene expression remains under the control of the respective endogenous promoter, and problems arising from transgene overexpression can be excluded.

Spliceosome-mediated RNA trans-splicing requires the coexistence of three distinct components: the spliceosome, target pre-mRNA transcripts, and pre-mRNA trans-splicing molecules (RTMs). The spliceosome and target pre-mRNA transcripts are naturally provided by the cells, while the RTMs are artificially engineered molecules that are able to bind specifically to target pre-mRNAs in the nucleus. There, they trigger the trans-splicing event between the endogenous pre-mRNA and the exogenous RTM in a process mediated by the spliceosome.

Each pre-mRNA trans-splicing molecule (RTM) is engineered to contain (a) a binding domain (BD) hybridizing to a selected target region to bring the RTM and target transcript into close proximity, (b) a splicing domain that includes splicing elements essential to mediate trans-splicing, and (c) a coding domain of a wild-type gene that is defective in the endogenous pre-mRNA. Similar to RNA cis-splicing processes, the binding domain and splicing domain sequences of the RTM are excised after trans-splicing and are not retained in the reprogrammed final mRNA products. Depending on the location of the mutation to be corrected or the region to be replaced, three types of SMART™ approaches are available. The two major types are replacement of upstream (5′) coding cassette (5′-trans-splicing) and replacement of a downstream (3′) coding cassette (3′-trans-splicing). The third type is internal exon(s) replacement.

### 3. Nucleic Acid OPA1 Trans-Splicing Molecules

In a first aspect, the invention provides OPA1 pre-mRNA trans-splicing molecules. More specifically, the invention provides a nucleic acid OPA1 trans-splicing molecule comprising, operatively linked in a 5′-to-3′ direction or in a 3′-to-5′ direction: (a) a binding domain configured to bind to a OPA1 target sequence comprising an intron, an intron/exon junction or an exon; (b) a splicing domain configured to mediate trans-splicing; and (c) a coding domain comprising functional OPA1 exons, wherein the nucleic acid OPA1 trans-splicing molecule is configured to trans-splice the coding domain to an endogenous OPA1 exon adjacent to the OPA1 target sequence, thereby replacing the endogenous OPA1 exons with the functional OPA1 exons and correcting a mutation in OPA1.

a. Human OPA1 Target Sequence. An OPA1 target sequence may be any fragment of the Homo sapiens OPA1 gene (Chromosome 3:193,593,144 to 193,697,811), which comprises an intron, an intron/exon junction or an exon.

However, the present invention is based, at least in part, on Applicant's discovery that a particular region of the human OPA1 genomic sequence provides highly efficient binding sites for binding domains of trans-splicing molecules and efficiently mediates trans-splicing. This targeted region warranties both the conservation and the trans-splicing of the eight OPA1 pre-mRNA transcripts, to correct the eight different OPA1 mRNAs and the corresponding OPA1 protein isoforms.

The human OPA1 trans-splicing targeted sequence is the Homo sapiens OPA1 intron 8-9 (193,637,282 to 193,637,951)/EXON 9 (ENSE00003604184: 193,637,952 to 193,638,065) genomic sequence. In certain embodiments, the Homo sapiens OPA1 intron 8-9/Exon 9 genomic sequence is the sequence set forth in SEQ ID NO: 1.

SEQ ID NO: 1
gtatgtgaaa aattgatagt gaacttgcca attagcaaaa aaagaagcag
cttagcttcc taaaaattat gtgtatatat gtacacatac acatatatac
atactagatg taggcattta tattttttat gtaatcttac atgttccaag
taatgtctta agcaatatta tttgactatt ttagttcatt ataaatatta
ataaatataa gtacattata tttatgagtt actgtatgtg ttataaagga
agatatttgg ctttatatgt cttaatatta gtaatattta aatactgaac
agtggattaa attagccata tgcgtgaaat ttaagctaat agaattgaaa
atgtgtttgt aaacagtaaa ctagctatgg aaaagattta tggaaagtta
ataacctggt tttagaaata ctggtttaaa ttagcacaag tttttaaaat
aaaaattagg ctataaacag tggatccagt tatagttttg ctgttcctat
tttcaatgtg cacacatgca tcaatcacca tttttttacg gattttaaaa
tattttttca cctgtagaaa ttttaaaaga ctaaaaaact cagagcagca
ttacaaatag gttttaattt taatttggta tcagaaaaat atgaataagt
gttctttgtt ttgtgggaag GTTGTTGTGG TTGGAGATCA GAGTGCTGGA
AAGACTAGTG TGTTGGAAAT GATTGCCCAA GCTCGAATAT TCCCAAGAGG
ATCTGGGGAG ATGATGACAC GTTCTCCAGT TAAG

In this 784-nucleotide (nt) sequence, Homo sapiens OPA1 intron 8-9 (670 bp) and Homo sapiens OPA1 EXON 9 (114 bp) are respectively indicated in lowercases and uppercases. The human OPA1 trans-splicing targeted sequence is located downstream to OPA1 alternate spliced exons (4, 4b, 5b) and upstream to the pathogenic variants observed in Hereditary Optic Neuropathies associated with mutations in the OPA1 gene.

Thus, in preferred embodiments of the present invention, the OPA1 target sequence comprising an intron, an intron/exon junction or an exon is the human OPA1 trans-splicing targeted sequence of SEQ ID NO: 1.

b. Binding Domains. A binding domain present in a nucleic acid OPA1 trans-splicing molecule according to the invention is able to bind specifically to the human OPA1 intron 8-9/Exon 9 target sequence of SEQ ID NO: 1, or to the complementary sequence thereof. As used herein, the term “specific binding” between a binding domain and the OPA1 target sequence refers to hydrogen bonding between the binding domain and the OPA1 target sequence in a degree sufficient to mediate trans-splicing by bringing the trans-splicing molecule into association with the target pre-mRNA. Preferably, the hydrogen bonds between the binding domain and the OPA1 target sequence are between nucleotide bases that are complementary to and in antisense orientation from one another (in which case the term “specific binding” refers to “specific hybridization” to one another). As will be understood by one skilled in the art, the term “specific binding” encompasses the binding the binding domain and a portion of the OPA1 target sequence. Such a portion is called herein “binding site”.

The binding domain may be 100% complementary to the OPA1 target sequence of SEQ ID NO: 1, or to the complementary sequence thereof, or have sufficient complementarity to be able to hybridize stably with the target pre-mRNA. For example, in some embodiments, the binding domain is 80%, 81%, 82%, 83%, 84%, 85%, 86%, 87%, 88%, 89%, 90%, 91%, 92%, 93%, 94%, 95%, 96%, 97%, 98%, 99%, or 100% complementary to the OPA1 target sequence of SEQ ID NO: 1, or to the complementary sequence thereof. The degree of complementarity is selected by one of skill in the art based on the need to keep the trans-splicing molecule and the nucleic acid construct containing the necessary sequences for expression and for inclusion in a recombinant adeno-associated virus within a 3,000 or up to 4,000 nucleotide base limit. The selection of this sequence and strength of hybridization depends on the complementarity and the length of the nucleic acid (See, for example, Sambrook et al., 1989, Molecular Cloning, A Laboratory Manual, 2d Ed., Cold Spring Harbor Laboratory Press, Cold Spring Harbor, N.Y.).

In the practice of the present invention, a binding domain can be 6 to 500 nucleotides in length. In some embodiments, the binding domain is from 20 to 400 nucleotides in length. In some embodiments, the binding domain is from 50 to 300 nucleotides in length. In some embodiments, the binding domain is from 100 to 200 nucleotides in length. In some embodiments, the binding domain is from 10-20 nucleotides in length (e.g., 10, 11, 12, 13, 14, 15, 16, 17, 18, 19, or 20 nucleotides in length), 20-30 nucleotides in length (e.g., 20, 21, 22, 23, 24, 25, 26, 27, 28, 29, or 30 nucleotides in length), 30-40 nucleotides in length (e.g., 30, 31, 32, 33, 34, 35, 36, 37, 38, 39, or 40 nucleotides in length), 40-50 nucleotides in length (e.g., 40, 41, 42, 43, 44, 45, 46, 47, 48, 49, 50 nucleotides in length), 50-60 nucleotides in length (e.g., 50, 51, 52, 53, 54, 55, 56, 57, 58, 59, or 60 nucleotides in length), 60-70 nucleotides in length (e.g., 60, 61, 62, 63, 64, 65, 66, 67, 68, 69, or 70 nucleotides in length), 70-80 nucleotides in length (e.g., 70, 71, 72, 73, 74, 75, 76, 77, 78, 79, or 80 nucleotides in length), 80-90 nucleotides in length (e.g., 80, 81, 82, 83, 84, 85, 86, 87, 88, 89, or 90 nucleotides in length), 90-100 nucleotides in length (e.g., 90, 91, 92, 93, 94, 95, 96, 97, 98, 99, or 100 nucleotides in length), 100-110 nucleotides in length (e.g., 100, 101, 102, 103, 104, 105, 106, 107, 108, 109, or 110 nucleotides in length), 110-120 nucleotides in length (e.g., 110, 111, 112, 113, 114, 115, 116, 117, 118, 119, or 120 nucleotides in length), 120-130 nucleotides in length (e.g., 120, 121, 122, 123, 124, 125, 126, 127, 128, 129, or 130 nucleotides in length), 130-140 nucleotides in length (e.g., 130, 131, 132, 133, 134, 135, 136, 137, 138, 139, or 140 nucleotides in length), 140-150 nucleotides in length (e.g., 140, 141, 142, 143, 144, 145, 146, 147, 148, 149, or 150 nucleotides in length), 150-160 nucleotides in length (e.g., 150, 151, 152, 153, 154, 155, 156, 157, 158, 159, or 160 nucleotides in length), 160-170 nucleotides in length (e.g., 160, 161, 162, 163, 164, 165, 166, 167, 168, 169, or 170 nucleotides in length), 170-180 nucleotides in length (e.g., 170, 171, 172, 173, 174, 175, 176, 177, 178, 179, or 180 nucleotides in length), 180-190 nucleotides in length (e.g., 180, 181, 182, 183, 184, 185, 186, 187, 188, 189, or 190 nucleotides in length), 190-200 nucleotides in length (e.g., 190, 191, 192, 193, 194, 195, 196, 197, 198, 199, or 200 nucleotides in length), 200-210 nucleotides in length (e.g., 200, 201, 202, 203, 204, 205, 206, 207, 208, 209 or 210 nucleotides in length), 210-220 nucleotides in length (e.g., 210, 211, 212, 213, 214, 215, 216, 217, 218, 219 or 220 nucleotides in length), 220-230 nucleotides in length (e.g., 220, 221, 222, 223, 224, 225, 226, 227, 228, 229 or 230 nucleotides in length), 230-240 nucleotides in length (e.g., 230, 231, 232, 233, 234, 235, 236, 237, 238, 239 or 240 nucleotides in length), 240-250 nucleotides in length (e.g., 240, 241, 242, 243, 244, 245, 246, 247, 248, 249 or 250 nucleotides in length), 250-260 nucleotides in length (e.g., 250, 251, 252, 253, 254, 255, 256, 257, 258, 259 or 260 nucleotides in length), 260-270 nucleotides in length (e.g., 260, 261, 262, 263, 264, 265, 266, 267, 268, 269 or 270 nucleotides in length), 270-280 nucleotides in length (e.g., 270, 271, 272, 273, 274, 275, 276, 277, 278, 279 or 280 nucleotides in length), 280-290 nucleotides in length (e.g., 280, 281, 282, 283, 284, 285, 286, 287, 288, 289 or 290 nucleotides in length), 290-300 nucleotides in length (e.g., 290, 291, 292, 293, 294, 295, 296, 297, 298, 299 or 300 nucleotides in length), 300-350 nucleotides in length, 350-400 nucleotides in length, 400-450 nucleotides in length, or 450-500 nucleotides in length.

In certain embodiments, the binding domain is a single binding domain, i.e., its sequence specifically hybridizes to a single portion (or binding site) of the OPA1 target sequence of SEQ ID NO: 1. In other embodiments, the binding domain contains sequences that specifically hybridize to more than one portion (or binding site) of the OPA1 target sequence of SEQ ID NO: 1. For example, the binding domain may be a double binding domain, specifically hybridizing to two distinct binding sites of the OPA1 target sequence of SEQ ID NO: 1, wherein the distinct binding sites are separated by tens or hundreds of nucleotides. More generally, the binding domain may be a multi-binding domain, specifically hybridizing to two or more distinct binding sites of the OPA1 target sequence of SEQ ID NO: 1, wherein the distinct binding sites are separated by tens or hundreds of nucleotides.

A binding site within the OPA1 target sequence of SEQ ID NO: 1 may be 6 to 300 nucleotides in length. For example, a binding site within the OPA1 target sequence of SEQ ID NO: 1 may comprise from 6 to 12, from 12 to 18, from 18 to 24, from 24 to 50, from 50 to 100, from 100 to 150, or from 150 to 200 nucleotides, or from 200 to 250 nucleotides or yet from 250 to 300 nucleotides.

In some embodiments, the binding domain of a nucleic acid OPA1 trans-splicing molecule according to the present invention is configured to bind (i.e., specifically binds to) the OPA1 target sequence at a binding site within or encompassing nucleotides 1 to 52 of SEQ ID NO: 1. Thus, in certain embodiments, the binding domain of a nucleic acid OPA1 trans-splicing molecule according to the present invention is complementary (e.g., antisense) to six or more consecutive nucleotides of a binding site within or encompassing nucleotides 1 to 52 of SEQ ID NO: 1. In particular, the binding domain may be complementary (e.g., antisense) to six or more consecutive nucleotides of a binding site within or encompassing nucleotides 1 to 50 of SEQ ID NO: 1. In certain embodiments, the binding site consists of nucleotides 1 to 52 of SEQ ID NO: 1. In certain embodiments, the binding site consists of nucleotides 1 to 50 of SEQ ID NO: 1.

In some embodiments, the binding domain of a nucleic acid OPA1 trans-splicing molecule according to the present invention specifically binds to the OPA1 target sequence at a binding site within or encompassing nucleotides 261 to 315 of SEQ I NO: 1. Thus, in certain embodiments, the binding domain of a nucleic acid OPA1 trans-splicing molecule according to the present invention is complementary (e.g., antisense) to six or more consecutive nucleotides of a binding site within or encompassing nucleotides 261 to 315 of SEQ ID NO: 1. In certain embodiments, the binding site consists of nucleotides 261 to 315 of SEQ ID NO: 1.

In some embodiments, the binding domain of a nucleic acid OPA1 trans-splicing molecule according to the present invention specifically binds to the OPA1 target sequence at a binding site within or encompassing nucleotides 361 to 603 of SEQ ID NO: 1, or at a binding site within or encompassing nucleotides 361 to 460 of SEQ ID NO: 1, or at a binding site within or encompassing nucleotides 375 to 460 of SEQ ID NO: 1 or at a binding site within or encompassing nucleotides 461 to 603 of SEQ ID NO: 1. Thus, in certain embodiments, the binding domain of a nucleic acid OPA1 trans-splicing molecule according to the present invention is complementary (e.g., antisense) to six or more consecutive nucleotides of a binding site within or encompassing nucleotides 361 to 603 of SEQ ID NO: 1. In particular, the binding domain may be complementary (e.g., antisense) to six or more consecutive nucleotides of a binding site within or encompassing nucleotides 361 to 460 of SEQ ID NO: 1, or of a binding site within or encompassing nucleotides 375 to 460 of SEQ ID NO: 1, or of a binding site within or encompassing nucleotides 461 to 603 of SEQ ID NO: 1. In certain embodiments, the binding site consists of nucleotides 361 to 603 of SEQ ID NO: 1. In other embodiments, the binding site consists of nucleotides 361 to 460 of SEQ ID NO: 1. In still other embodiments, the binding site consists of nucleotides 375 to 460 of SEQ ID NO: 1. In yet other embodiments, the binding site consists of nucleotides 461 to 603 of SEQ ID NO: 1.

In some embodiments, the binding domain of a nucleic acid OPA1 trans-splicing molecule according to the present invention specifically binds to the OPA1 target sequence at a binding site within or encompassing nucleotides 510 to 784 of SEQ ID NO: 1, or at a binding site within or encompassing nucleotides 510 to 756 of SEQ ID NO 1, or at a binding site within or encompassing nucleotides 510 to 726 of SEQ ID NO 1, or at a binding site within or encompassing nucleotides 518 to 784 of SEQ ID NO 1, or at a binding site within or encompassing nucleotides 518 to 756 of SEQ ID NO 1, or at a binding site within or encompassing nucleotides 518 to 726 of SEQ ID NO 1, or at a binding site within or encompassing nucleotides 561 to 784 of SEQ ID NO 1, or at a binding site within or encompassing nucleotides 561 to 756 of SEQ ID NO 1, or at a binding site within or encompassing nucleotides 561 to 726 of SEQ ID NO 1, or at a binding site within or encompassing nucleotides 627 to 784 of SEQ ID NO 1, or at a binding site within or encompassing nucleotides 627 to 756 of SEQ ID NO 1, or at a binding site within or encompassing nucleotides 627 to 726 of SEQ ID NO 1. Thus, in certain embodiments, the binding domain of a nucleic acid OPA1 trans-splicing molecule according to the present invention is complementary (e.g., antisense) to six or more consecutive nucleotides of a binding site within or encompassing nucleotides 510 to 784 of SEQ ID NO: 1. In particular, the binding domain may be complementary (e.g., antisense) to six or more consecutive nucleotides of a binding site within or encompassing nucleotides 510 to 756 of SEQ ID NO 1, or nucleotides 510 to 726 of SEQ ID NO 1, or nucleotides 518 to 784 of SEQ ID NO 1, or nucleotides 518 to 756 of SEQ ID NO 1, or nucleotides 518 to 726 of SEQ ID NO 1, or nucleotides 561 to 784 of SEQ ID NO 1, or nucleotides 561 to 756 of SEQ ID NO 1, or nucleotides 561 to 726 of SEQ ID NO 1, or nucleotides 627 to 784 of SEQ ID NO 1, or nucleotides 627 to 756 of SEQ ID NO 1, or nucleotides 627 to 726 of SEQ ID NO 1. In certain embodiments, the binding site consists of nucleotides 510 to 784 of SEQ ID NO: 1. In other embodiments, the binding site consists of nucleotides 510 to 756 of SEQ ID NO 1, or nucleotides 510 to 726 of SEQ ID NO 1, or nucleotides 518 to 784 of SEQ ID NO 1, or nucleotides 518 to 756 of SEQ ID NO 1, or nucleotides 518 to 726 of SEQ ID NO 1, or nucleotides 561 to 784 of SEQ ID NO 1, or nucleotides 561 to 756 of SEQ ID NO 1, or nucleotides 561 to 726 of SEQ ID NO 1, or nucleotides 627 to 784 of SEQ ID NO 1, or nucleotides 627 to 756 of SEQ ID NO 1, or nucleotides 627 to 726 of SEQ ID NO 1.

In some preferred embodiments, a binding domain of a nucleic acid OPA1 trans-splicing molecule according to the present invention comprises, or consists of, the single binding domain with the following 55-nt sequence (3′ to 5′): ctaatttaatccactgttcagtatttaaatattactaatattaagacatataaag (SEQ ID NO: 2-BD #114), which hybridizes to the OPA1 target sequence at a binding site consisting of nucleotides 261 to 315 of SEQ ID NO: 1. In certain embodiments, the binding domain is a fragment of SEQ ID NO: 2.

The term “fragment”, as used herein in relation to a binding domain refers to a portion of said binding domain which consists of consecutive nucleotides of the binding domain and which hybridizes to a binding site of the OPA1 target sequence of SEQ ID NO: 1. In other words, a fragment of a binding domain is a binding domain as defined herein.

In some preferred embodiments, a binding domain of a nucleic acid OPA1 trans-splicing molecule according to the present invention comprises, or consists of, the single binding domain with the following 86-nt sequence (3′ to 5′): cctaatttttattttaaaaacttgtgctaatttaaaccagtatttctaaaaccaggttattaac tttccataaatcttttccatag (SEQ ID NO: 3-BD #128), which hybridizes to the OPA1 target sequence at a binding site consisting of nucleotides 375 to 460 of SEQ ID NO:1. In certain embodiments, the binding domain is a fragment of SEQ ID NO: 3-BD #128.

In other preferred embodiments, a binding domain of a nucleic acid OPA1 trans-splicing molecule according to the present invention comprises, or consists of, the single binding domain with the following 143-nt sequence (3′ to 5′): taatgctgctctgagttttttagtcttttaaaatttctacaggtgaaaaaatattttaaaatcc gtaaaaaaatggtgattgatgcatgtgtgcacattgaaaataggaacagcaaaactataactgga tccactgtttatag (SEQ ID NO: 4-BD #162), which hybridizes to the OPA1 target sequence at a binding site consisting of nucleotides 461 to 603 of SEQ ID NO: 1. In certain embodiments, the binding domain is a fragment of SEQ ID NO: 4-BD #162.

In yet other preferred embodiments, a binding domain of a nucleic acid OPA1 trans-splicing molecule according to the present invention comprises, or consists of, the binding domain with the following 105-nt sequence (1× antisense+1× sense): ctgcttctttttttgctaattggcaagttcactatcaatttttcacatacctttatatgtctta atattagtaatatttaaatactgaacagtggattaaattag (SEQ ID NO: 5-BD #135), which hybridizes to the OPA1 target sequence at a binding site consisting of nucleotides 1 to 50 of SEQ ID NO: 1. In certain embodiments, the binding domain is a fragment of SEQ ID NO: 5-BD #135.

In still other preferred embodiments, a binding domain of a nucleic acid OPA1 trans-splicing molecule according to the present invention comprises, or consists of, the multiple binding domain with the following 250-nt sequence (4× antisense+1× sense): cctaatttttattttaaaaacttgtgctaatttaaaccagtatttctaaaaccaggttattaac tttccataaatcttttccatagctgcttctttttttgctaattggcaagttcactatcaatttt tcacatacctttatatgtcttaatattagtaatatttaaatactgaacagtggattaaattagc ttaaatttcacgcatatggctagtttactgtttacaaacacattttcaattctattag (SEQ ID NO: 6-BD #106), which hybridizes to the OPA1 target sequence at a first binding site consisting of nucleotides 375 to 460 of SEQ ID NO: 1 (like BD #128), at a second binding site consisting of nucleotides 1 to 50 of SEQ ID NO: 1 (like BD #135), to a third binding site consisting of nucleotides 316 to 335 of SEQ ID NO: 1, and to a fourth binding site consisting of nucleotides 336 to 374 of SEQ ID NO: 1.

In certain embodiments, a binding domain of a nucleic acid OPA1 trans-splicing molecule according to the present invention comprises, or consists of a combination of at least two of the binding domains described above, i.e., BD #114 (SEQ ID NO: 2), BD #128 (SEQ ID NO: 3), BD #162 (SEQ ID NO: 4), BD #135 (SEQ ID NO: 5), and BD #106 (SEQ ID NO: 6). For example, the binding domain may be a combination of BD #114 (SEQ ID NO: 2) and BD #128 (SEQ ID NO: 3), or of BD #114 (SEQ ID NO: 2) and BD #162 (SEQ ID NO: 4), or of BD #128 (SEQ ID NO: 3) and BD #162 (SEQ ID NO: 4), etc.,

Other antisense binding domains have also been generated by rational PCR.

Thus, in certain preferred embodiments, a binding domain of a nucleic acid OPA1 trans-splicing molecule according to the present invention comprises, or consists of, the binding domain with the following 100-nt sequence (3′ to 5′): GCAATCATTTCCAACACACTAGTCTTTCCAGCACTCTGATCTCCAACCACAACAACcttcccac aaaacaaagaacacttattcatatttttctgatacc (SEQ ID NO: 7-BD #100), which hybridizes to the OPA1 target sequence at a binding site consisting of nucleotides 627 to 726 of SEQ ID NO: 1. In certain embodiments, the binding domain is a fragment of SEQ ID NO: 7.

In some preferred embodiments, a binding domain of a nucleic acid OPA1 trans-splicing molecule according to the present invention comprises, or consists of, the binding domain with the following 130-nt sequence (3′ to 5′): CCAGATCCTCTTGGGAATATTCGAGCTTGGGCAATCATTTCCAACACACTAGTCTTTCCAGCAC TCTGATCTCCAACCACAACAACcttcccacaaaacaaagaacacttattcatatttttctgata cc (SEQ ID NO: 8-BD #130), which hybridizes to the OPA1 target sequence at a binding site consisting of nucleotides 627 to 756 of SEQ ID NO: 1. In certain embodiments, the binding domain is a fragment of SEQ ID NO: 8.

In some preferred embodiments, a binding domain of a nucleic acid OPA1 trans-splicing molecule according to the present invention comprises, or consists of, the binding domain with the following 158-nt sequence (3′ to 5′): CTTAACTGGAGAACGTGTCATCATCTCCCCAGATCCTCTTGGGAATATTCGAGCTTGGGCAATC ATTTCCAACACACTAGTCTTTCCAGCACTCTGATCTCCAACCACAACAACcttcccacaaaaca aagaacacttattcatatttttctgatacc (SEQ ID NO: 9-BD #158), which hybridizes to the OPA1 target sequence at a binding site consisting of nucleotides 627 to 784 of SEQ ID NO: 1. In certain embodiments, the binding domain is a fragment of SEQ ID NO: 9.

In some preferred embodiments, a binding domain of a nucleic acid OPA1 trans-splicing molecule according to the present invention comprises, or consists of, the binding domain with the following 166-nt sequence (3′ to 5′): GCAATCATTTCCAACACACTAGTCTTTCCAGCACTCTGATCTCCAACCACAACAACcttcccac aaaacaaagaacacttattcatatttttctgataccaaattaaaattaaaacctatttgtaatg ctgctctgagttttttagtcttttaaaatttctacagg (SEQ ID NO: 10-BD #166), which hybridizes to the OPA1 target sequence at a binding site consisting of nucleotides 561 to 726 of SEQ ID NO: 1. In certain embodiments, the binding domain is a fragment of SEQ ID NO: 10.

In some preferred embodiments, a binding domain of a nucleic acid OPA1 trans-splicing molecule according to the present invention comprises, or consists of, the binding domain with the following 196-nt sequence (3′ to 5′): CCAGATCCTCTTGGGAATATTCGAGCTTGGGCAATCATTTCCAACACACTAGTCTTTCCAGCAC TCTGATCTCCAACCACAACAACcttcccacaaaacaaagaacacttattcatatttttctgata ccaaattaaaattaaaacctatttgtaatgctgctctgagttttttagtcttttaaaatttcta cagg (SEQ ID NO: 11-BD #196), which hybridizes to the OPA1 target sequence at a binding site consisting of nucleotides 561 to 756 of SEQ ID NO: 1. In certain embodiments, the binding domain is a fragment of SEQ ID NO: 11.

In some preferred embodiments, a binding domain of a nucleic acid OPA1 trans-splicing molecule according to the present invention comprises, or consists of, the binding domain with the following 224-nt sequence (3′ to 5′): CTTAACTGGAGAACGTGTCATCATCTCCCCAGATCCTCTTGGGAATATTCGAGCTTGGGCAATC ATTTCCAACACACTAGTCTTTCCAGCACTCTGATCTCCAACCACAACAACcttcccacaaaaca aagaacacttattcatatttttctgataccaaattaaaattaaaacctatttgtaatgctgctc tgagttttttagtcttttaaaatttctacagg (SEQ ID NO: 12-BD #224), which hybridizes to the OPA1 target sequence at a binding site consisting of nucleotides 561 to 784 of SEQ ID NO: 1. In certain embodiments, the binding domain is a fragment of SEQ ID NO: 12.

In some preferred embodiments, a binding domain of a nucleic acid OPA1 trans-splicing molecule according to the present invention comprises, or consists of, the binding domain with the following 209-nt sequence (3′ to 5′): GCAATCATTTCCAACACACTAGTCTTTCCAGCACTCTGATCTCCAACCACAACAACcttcccac aaaacaaagaacacttattcatatttttctgataccaaattaaaattaaaacctatttgtaatg ctgctctgagttttttagtcttttaaaatttctacaggtgaaaaaatattttaaaatccgtaaa aaaatggtgattgatgc (SEQ ID NO: 13-BD #209), which hybridizes to the OPA1 target sequence at a binding site consisting of nucleotides 518 to 726 of SEQ ID NO: 1. In certain embodiments, the binding domain is a fragment of SEQ ID NO: 13.

In some preferred embodiments, a binding domain of a nucleic acid OPA1 trans-splicing molecule according to the present invention comprises, or consists of, the binding domain with the following 239-nt sequence (3′ to 5′): CCAGATCCTCTTGGGAATATTCGAGCTTGGGCAATCATTTCCAACACACTAGTCTTTCCAGCAC TCTGATCTCCAACCACAACAACcttcccacaaaacaaagaacacttattcatatttttctgata ccaaattaaaattaaaacctatttgtaatgctgctctgagttttttagtcttttaaaatttcta caggtgaaaaaatattttaaaatccgtaaaaaaatggtgattgatgc (SEQ ID NO: 14-BD #239), which hybridizes to the OPA1 target sequence at a binding site consisting of nucleotides 518 to 756 of SEQ ID NO: 1. In certain embodiments, the binding domain is a fragment of SEQ ID NO: 14.

In some preferred embodiments, a binding domain of a nucleic acid OPA1 trans-splicing molecule according to the present invention comprises, or consists of, the binding domain with the following 267-nt sequence (3′ to 5′): CTTAACTGGAGAACGTGTCATCATCTCCCCAGATCCTCTTGGGAATATTCGAGCTTGGGCAATC ATTTCCAACACACTAGTCTTTCCAGCACTCTGATCTCCAACCACAACAACcttcccacaaaaca aagaacacttattcatatttttctgataccaaattaaaattaaaacctatttgtaatgctgctc tgagttttttagtcttttaaaatttctacaggtgaaaaaatattttaaaatccgtaaaaaaatg gtgattgatgc (SEQ ID NO: 15-BD #267), which hybridizes to the OPA1 target sequence at a binding site consisting of nucleotides 518 to 784 of SEQ ID NO: 1. In certain embodiments, the binding domain is a fragment of SEQ ID NO: 15.

In some preferred embodiments, a binding domain of a nucleic acid OPA1 trans-splicing molecule according to the present invention comprises, or consists of, the binding domain with the following 217-nt sequence (3′ to 5′): gcaatcatttccaacacactagtctttccagcactctgatctccaaccacaacaaccttcccac aaaacaaagaacacttattcatatttttctgataccaaattaaaattaaaacctatttgtaatg ctgctctgagttttttagtcttttaaaatttctacaggtgaaaaaatattttaaaatccgtaaa aaaatggtgattgatgcatgtgtgc (SEQ ID NO: 16-BD #217), which hybridizes to the OPA1 target sequence at a binding site consisting of nucleotides 510 to 726 of SEQ ID NO: 1. In certain embodiments, the binding domain is a fragment of SEQ ID NO: 16.

In some preferred embodiments, a binding domain of a nucleic acid OPA1 trans-splicing molecule according to the present invention comprises, or consists of, the binding domain with the following 247-nt sequence (3′ to 5′): CCAGATCCTCTTGGGAATATTCGAGCTTGGGCAATCATTTCCAACACACTAGTCTTTCCAGCAC TCTGATCTCCAACCACAACAACcttcccacaaaacaaagaacacttattcatatttttctgata ccaaattaaaattaaaacctatttgtaatgctgctctgagttttttagtcttttaaaatttcta caggtgaaaaaatattttaaaatccgtaaaaaaatggtgattgatgcatgtgtgc (SEQ ID NO: 17-BD #247), which hybridizes to the OPA1 target sequence at a binding site consisting of nucleotides 510 to 756 of SEQ ID NO: 1. In certain embodiments, the binding domain is a fragment of SEQ ID NO: 17.

In some preferred embodiments, a binding domain of a nucleic acid OPA1 trans-splicing molecule according to the present invention comprises, or consists of, the binding domain with the following 275-nt sequence (3′ to 5′): CTTAACTGGAGAACGTGTCATCATCTCCCCAGATCCTCTTGGGAATATTCGAGCTTGGGCAATC ATTTCCAACACACTAGTCTTTCCAGCACTCTGATCTCCAACCACAACAACcttcccacaaaaca aagaacacttattcatatttttctgataccaaattaaaattaaaacctatttgtaatgctgctc tgagttttttagtcttttaaaatttctacaggtgaaaaaatattttaaaatccgtaaaaaaatg gtgattgatgcatgtgtgc (SEQ ID NO: 18-BD #275), which is hybridizes to the OPA1 target sequence at a binding site consisting of nucleotides 510 to 784 of SEQ ID NO: 1. In certain embodiments, the binding domain is a fragment of SEQ ID NO: 18.

c. Splicing Domain. A nucleic acid OPA1 trans-splicing molecule also comprises a splicing domain. The terms “splicing domain”, and “splice region” are used herein interchangeably. They refer to a nucleic acid sequence having motifs that are recognized by the spliceosome and that mediate trans-splicing.

In a 3′-trans-splicing molecule such as a nucleic acid OPA1 trans-splicing molecule according to the present invention, a splicing domain comprises a branch point (BP), a polypyrimidine tract (PPT), and an acceptor splice site mediating trans-splicing. The splice site is a 3′ acceptor splice site (AG or YAG).

Splicing domains may be selected by one skilled in the art according to known methods and principles. Consensus sequences for splicing domains used in RNA splicing are well known in the art (See Moore et al., 1993, The RNA World, Cold Spring Harbor Laboratory Press, p. 303-358). However, modified consensus sequences that maintain the ability to function as a splicing domain may be used in the practice of the present invention.

In certain embodiments, a splicing domain of a nucleic acid OPA1 trans-splicing molecule according to the present invention comprises a branch point (BP), a polypyrimidine tract (PPT) and a 3′ acceptor splice site, wherein the PPT is located between the branch point and the splice site.

In certain embodiments, the branch point consists of the 8-nucleotides (nt) nucleic acid sequence: 5′-tactaact-3′ (SEQ ID NO: 19).

In certain embodiments, the polypyrimidine tract consists of the 16-nt nucleic acid sequence: 5′-tcttcttttttttctg-3′ (SEQ ID NO: 20).

In certain embodiments, the 3′ acceptor splice site consists of 3-nt the nucleic acid sequence: 5′-cag-3′ (SEQ ID NO: 21).

The splicing domain may be included as part of an intronic splicing enhancer (ISE), which may include one or more additional components. For example, a spacer region may be present within an intronic splicing enhancer (ISE) to separate the splicing domain from the binding domain in the pre-mRNA trans-splicing molecule. The spacer region may be designed to include features such as (1) stop codons which function to block translation of any unspliced trans-splicing molecule and/or (2) sequences that enhance trans-splicing to the target pre-mRNA. The spacer may be 3 to 30 nucleotides or more depending on the lengths of the other components of the trans-splicing molecule.

In certain embodiments, the spacer consists of the following 27-nt nucleic acid sequence: 5′-gagaacattattatagcgttgctcgag-3′ (SEQ ID NO: 22).

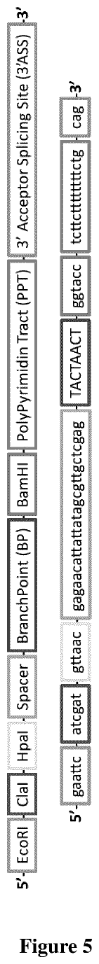

In certain embodiments, the binding domain of a nucleic acid OPA1 trans-splicing molecule according to the present invention is linked to an Intronic Splicing Enhancer cassette, which contains the 27-nt spacer, the 8-nt branch point, the 16-nt polypyrimidine tract and the 3-nt 3′ acceptor splice site, defined above. For example, such an Intronic Splicing Enhancer cassette is as presented in FIG. 5, and consists of the following nucleic acid sequence:

(SEQ ID NO: 23)
5′-gagaacattattatagcgttgctcgagtactaactggtacctcttc
ttttttttctgcag-3′

d. Coding Domain. The third component present in a nucleic acid OPA1 pre-mRNA trans-splicing molecule according to the present invention is a coding domain. The term “coding domain”, as used herein, refers to a sequence (e.g., a wild-type or corrected coding sequence of interest) configured to be trans-spliced onto the target OPA1 endogenous pre-mRNA, to replace one or more endogenous, mutated exons in the target pre-mRNA. Preferably, the coding domain comprises a wild-type or corrected coding sequence (usually corresponding to one or more functional OPA1 exons) that is necessary to repair the targeted mutation(s) or defect(s) that cause(s) diseases or disorders associated with OPA1 mutations, in particular the hereditary optic neuropathy of interest such as ADOA, ADOA+ and Behr′ syndrome. Thus, in one embodiment, the coding domain comprises a single exon of the target OPA1 gene, which contains the normal, wild-type sequence lacking the disease-causing mutation. In another embodiment, the coding domain comprises multiple exons of the target OPA1 gene, each exon containing the normal, wild-type sequence lacking the disease-causing mutation.

In some embodiments of the present invention, the coding domain includes complementary DNA (cDNA). For example, one or more functional OPA1 exons within the coding domain can be a cDNA sequence. In some embodiments, the entire coding domain is a cDNA sequence. Additionally, or alternatively, all or a portion of the coding domain, or one or more functional OPA1 exons thereof, can be a naturally-occurring (i.e., wild-type) sequence (e.g., a sequence having 90%, 91%, 92%, 93%, 94%, 95%, 96%, 97%, 98%, 99%, or 100% sequence identity with an endogenous, normal OPA1 exon.

All or a portion of the coding domain, or one or more functional OPA1 exons thereof, is a codon optimized sequence. Codon optimization refers to modifying a nucleic acid sequence to change individual nucleic acids without any resulting change in the encoded amino acid. Sequences modified in this way are referred to herein as “codon-optimized” in which a nucleic acid sequence has been modified, e.g., to enhance expression or stability, without resulting in a change in the encoded amino acid. Codon optimization is known in the art (see for example, U.S. Pat. Nos. 7,561,972, 7,561,973, and 7,888,112). The sequence surrounding the translation start site can be converted to a consensus Kozak sequence according to known methods (See, e.g., Kozak et al., Nucleic Acids Res., 1987, 15 (20): 8125-8148).

For delivery via a recombinant Adeno-Associated Viral (AAV) vector, the coding domain can be a nucleic acid sequence of up to 4,000 nucleotide bases in length (e.g., from 1,000 to 4,000 nucleotide bases in length, from 1,500 to 3,000 nucleotide bases in length, or from 2,000 to 2,500 nucleotide bases in length).

In preferred embodiments, the coding domain of a nucleic acid OPA1 3′-pre-mRNA trans-splicing molecule according to the present invention comprises functional OPA1 exons 9-31, i.e., all the exons that are 3′ to the binding region of the binding domain to the OPA1 target (i.e., of intron 8-9). In certain embodiments, the coding domain comprises the following 2013-nt nucleic acid sequence (an optimized and corrective OPA1 cDNA, which corresponds to the wild-type Homo sapiens OPA1 amino acid sequence, encoded by Exon9 to the Stop codon) (in 5′-3′):

(SEQ ID NO: 24)
GTAGTAGTCGTAGGTGATCAGAGTGCTGGAAAGACTAGTGTGTTGGAAATGATTGCCCAAGCTCGAA
TATTCCCAAGAGGATCTGGGGAGATGATGACACGTTCTCCAGTTAAGGTGACTCTGAGTGAAGGTCC
TCACCATGTGGCCCTATTTAAAGATAGTTCTCGGGAGTTTGATCTTACCAAAGAAGAAGATCTTGCA
GCATTAAGACATGAAATAGAACTTCGAATGAGGAAAAATGTGAAAGAAGGCTGTACCGTTAGCCCTG
AGACCATATCCTTAAATGTAAAAGGCCCTGGACTACAGAGGATGGTGCTTGTTGACTTACCAGGTGT
GATTAATACTGTGACATCAGGCATGGCTCCTGACACAAAGGAAACTATTTTCAGTATCAGCAAAGCT
TACATGCAGAATCCTAATGCCATCATACTGTGTATTCAAGATGGATCTGTGGATGCTGAACGCAGTA
TTGTTACAGACTTGGTCAGTCAAATGGACCCTCATGGAAGGAGAACCATATTCGTTTTGACCAAAGT
AGACCTGGCAGAGAAAAATGTAGCCAGTCCAAGCAGGATTCAGCAGATAATTGAAGGAAAGCTCTTC
CCAATGAAAGCTTTAGGTTATTTTGCTGTTGTAACAGGAAAAGGGAACAGCTCTGAAAGCATTGAAG
CTATAAGAGAATATGAAGAAGAGTTTTTTCAGAATTCAAAGCTCCTAAAGACAAGCATGCTAAAGGC
ACACCAAGTGACTACAAGAAATTTAAGCCTTGCAGTATCAGACTGCTTTTGGAAAATGGTACGAGAG
TCTGTTGAACAACAGGCTGATAGTTTCAAAGCAACACGTTTTAACCTTGAAACTGAATGGAAGAATA
ACTATCCTCGCCTGCGGGAACTTGACCGGAATGAACTATTTGAAAAAGCTAAAAATGAAATCCTTGA
TGAAGTTATCAGTCTGAGCCAGGTTACACCAAAACATTGGGAGGAAATCCTTCAACAATCTTTGTGG
GAAAGAGTATCAACTCATGTGATTGAAAACATCTACCTTCCAGCTGCGCAGACCATGAATTCAGGAA
CTTTTAACACCACAGTGGATATCAAGCTTAAACAGTGGACTGATAAACAACTTCCTAATAAAGCAGT
AGAGGTTGCTTGGGAGACCCTACAAGAAGAATTTTCCCGCTTTATGACAGAACCGAAAGGGAAAGAG
CATGATGACATATTTGATAAACTTAAAGAGGCTGTTAAGGAAGAAAGTATTAAACGACACAAGTGGA
ATGACTTTGCGGAGGACAGCTTGAGGGTTATTCAACACAATGCTTTGGAAGACCGATCCATATCTGA
TAAACAGCAATGGGATGCAGCTATTTATTTTATGGAAGAGGCTCTGCAGGCTCGTCTCAAGGATACT
GAAAATGCAATTGAAAACATGGTGGGTCCAGACTGGAAAAAGAGGTGGTTATACTGGAAGAATCGGA
CCCAAGAACAGTGTGTTCACAATGAAACCAAGAATGAATTGGAGAAGATGTTGAAATGTAATGAGGA
GCACCCAGCTTATCTTGCAAGTGATGAAATAACCACAGTCCGGAAGAACCTTGAATCCCGAGGAGTA
GAAGTAGATCCAAGCTTGATTAAGGATACTTGGCATCAAGTTTATAGAAGACATTTTTTAAAAACAG
CTCTAAACCATTGTAACCTTTGTCGAAGAGGTTTTTATTACTACCAAAGGCATTTTGTAGATTCTGA
GTTGGAATGCAATGATGTGGTCTTGTTTTGGCGTATACAGCGCATGCTTGCTATCACCGCAAATACT
TTAAGGCAACAACTTACAAATACTGAAGTTAGGCGATTAGAGAAAAATGTTAAAGAGGTATTGGAAG
ATTTTGCTGAAGATGGTGAGAAGAAGATTAAATTGCTTACTGGTAAACGCGTTCAACTGGCGGAAGA
CCTCAAGAAAGTTAGAGAAATTCAAGAAAAACTTGATGCTTTCATTGAAGCTCTTCATCAGGAGAAA
TAA,

or a codon-optimized version thereof.

This coding domain may be made compatible with the Intronic Splicing Enhancer cassette described above (see SEQ ID NO: 25 presented in the Examples below)

e. Optional Components and Modifications of a Nucleic Acid OPA1 pre-mRNA Trans-Splicing Molecule. In certain embodiments, a nucleic acid OPA1 trans-splicing molecule according to the present invention comprises 3′UTR sequences or ribozyme sequences added to the 3′ or 5′ end.

In some embodiments, splicing enhancers such as, for example, sequences referred to as exonic splicing enhancers may also be included in the structure of synthetic nucleic acid OPA1 pre-mRNA trans-splicing molecules according to the present invention. Additional features can be added to a pre-mRNA trans-splicing molecule, such as polyadenylation signals to modify RNA expression/stability, additional binding regions, “safety”-self complementary regions, additional splice sites, or protective groups to modulate the stability of the molecule and prevent degradation. In addition, stop codons may be included in the pre-mRNA trans-splicing molecule structure to prevent translation of unspliced pre-mRNA trans-splicing molecules. Further elements such as a 3′ hairpin structure, circularized RNA, nucleotide base modification, or synthetic analogs can be incorporated into a nucleic acid OPA1 pre-mRNA trans-splicing molecule according to the present invention to promote or facilitate nuclear localization and spliceosomal incorporation, and intra-cellular stability.

When nucleic acid OPA1 pre-mRNA trans-splicing molecules are synthesized in vitro, such trans-splicing molecules can be modified at the base moiety, sugar moiety, or phosphate backbone, for example, to improve stability of the molecule, hybridization to the target pre-mRNA, transport into the cell and/or nucleus, stability in the cells to enzymatic cleavage, etc. For example, modification of a pre-mRNA trans-splicing molecule to reduce the overall charge can enhance the cellular uptake of the molecule. In addition, modifications can be made to reduce susceptibility to nuclease or chemical degradation. The nucleic acid OPA1 pre-mRNA trans-splicing molecules may be synthesized in such a way as to be conjugated to another molecule, e.g., a peptide, hybridization triggered cross-linking agent, transport agent, hybridization-triggered cleavage agent, etc.

Various other well-known modifications to the nucleic acid molecules can be introduced as a means of increasing intracellular stability and half-life. Possible modifications are known to the art. Modifications, which may be made to the structure of synthetic pre-mRNA trans-splicing molecules include backbone modifications.

f. Specific Nucleic Acid OPA1 pre-mRNA Trans-Splicing Molecule. The present invention provides several specific nucleic acid OPA1 pre-mRNA trans-splicing molecules, each containing one of the Binding Domains defined above. In particular, the present invention provides several specific nucleic acid OPA1 pre-mRNA trans-splicing molecules, whose sequence, in the 5′-to-3′ direction, consists of: one of the specific Binding Domains defined above, the 27-nt spacer, the 8-nt Branch Point, KpnI (a restriction cloning site), the 16-nt polypyrimidine tract, the 3-nt 3′ acceptor splice site, and the Coding Domain corresponding to the 2013-nt wild-type or codon-optimized Homo sapiens OPA1 cDNA from Exon9 to the STOP codon, as defined above.

The specific nucleic acid OPA1 trans-splicing molecules are:
The 2326-nt sequence SEQ ID NO: 26 (designed as OPA1 RTMₜₕ#106 in the Examples section):

CCTAATTTTTATTTTAAAAACTTGTGCTAATTTAAACCAGTATTTCTAAAACCAGGTTATTAAC
TTTCCATAAATCTTTTCCATAGCTGCTTCTTTTTTTGCTAATTGGCAAGTTCACTATCAATTTT
TCACATACCTTTATATGTCTTAATATTAGTAATATTTAAATACTGAACAGTGGATTAAATTAGC
TTAAATTTCACGCATATGGCTAGTTTACTGTTTACAAACACATTTTCAATTCTATTAGAACGAG
AACATTATTATAGCGTTGCTCGAGTACTAACTGGTACCTCTTCTTTTTTTTCTGCAGGTAGTAGT
CGTAGGTGATCAGAGTGCTGGAAAGACTAGTGTGTTGGAAATGATTGCCCAAGCTCGAATATTCCCA
AGAGGATCTGGGGAGATGATGACACGTTCTCCAGTTAAGGTGACTCTGAGTGAAGGTCCTCACCATG
TGGCCCTATTTAAAGATAGTTCTCGGGAGTTTGATCTTACCAAAGAAGAAGATCTTGCAGCATTAAG
ACATGAAATAGAACTTCGAATGAGGAAAAATGTGAAAGAAGGCTGTACCGTTAGCCCTGAGACCATA
TCCTTAAATGTAAAAGGCCCTGGACTACAGAGGATGGTGCTTGTTGACTTACCAGGTGTGATTAATA
CTGTGACATCAGGCATGGCTCCTGACACAAAGGAAACTATTTTCAGTATCAGCAAAGCTTACATGCA
GAATCCTAATGCCATCATACTGTGTATTCAAGATGGATCTGTGGATGCTGAACGCAGTATTGTTACA
GACTTGGTCAGTCAAATGGACCCTCATGGAAGGAGAACCATATTCGTTTTGACCAAAGTAGACCTGG
CAGAGAAAAATGTAGCCAGTCCAAGCAGGATTCAGCAGATAATTGAAGGAAAGCTCTTCCCAATGAA
AGCTTTAGGTTATTTTGCTGTTGTAACAGGAAAAGGGAACAGCTCTGAAAGCATTGAAGCTATAAGA
GAATATGAAGAAGAGTTTTTTCAGAATTCAAAGCTCCTAAAGACAAGCATGCTAAAGGCACACCAAG
TGACTACAAGAAATTTAAGCCTTGCAGTATCAGACTGCTTTTGGAAAATGGTACGAGAGTCTGTTGA
ACAACAGGCTGATAGTTTCAAAGCAACACGTTTTAACCTTGAAACTGAATGGAAGAATAACTATCCT
CGCCTGCGGGAACTTGACCGGAATGAACTATTTGAAAAAGCTAAAAATGAAATCCTTGATGAAGTTA
TCAGTCTGAGCCAGGTTACACCAAAACATTGGGAGGAAATCCTTCAACAATCTTTGTGGGAAAGAGT
ATCAACTCATGTGATTGAAAACATCTACCTTCCAGCTGCGCAGACCATGAATTCAGGAACTTTTAAC
ACCACAGTGGATATCAAGCTTAAACAGTGGACTGATAAACAACTTCCTAATAAAGCAGTAGAGGTTG
CTTGGGAGACCCTACAAGAAGAATTTTCCCGCTTTATGACAGAACCGAAAGGGAAAGAGCATGATGA
CATATTTGATAAACTTAAAGAGGCTGTTAAGGAAGAAAGTATTAAACGACACAAGTGGAATGACTTT
GCGGAGGACAGCTTGAGGGTTATTCAACACAATGCTTTGGAAGACCGATCCATATCTGATAAACAGC
AATGGGATGCAGCTATTTATTTTATGGAAGAGGCTCTGCAGGCTCGTCTCAAGGATACTGAAAATGC
AATTGAAAACATGGTGGGTCCAGACTGGAAAAAGAGGTGGTTATACTGGAAGAATCGGACCCAAGAA
CAGTGTGTTCACAATGAAACCAAGAATGAATTGGAGAAGATGTTGAAATGTAATGAGGAGCACCCAG
CTTATCTTGCAAGTGATGAAATAACCACAGTCCGGAAGAACCTTGAATCCCGAGGAGTAGAAGTAGA
TCCAAGCTTGATTAAGGATACTTGGCATCAAGTTTATAGAAGACATTTTTTAAAAACAGCTCTAAAC
CATTGTAACCTTTGTCGAAGAGGTTTTTATTACTACCAAAGGCATTTTGTAGATTCTGAGTTGGAAT
GCAATGATGTGGTCTTGTTTTGGCGTATACAGCGCATGCTTGCTATCACCGCAAATACTTTAAGGCA
ACAACTTACAAATACTGAAGTTAGGCGATTAGAGAAAAATGTTAAAGAGGTATTGGAAGATTTTGCT
GAAGATGGTGAGAAGAAGATTAAATTGCTTACTGGTAAACGCGTTCAACTGGCGGAAGACCTCAAGA
AAGTTAGAGAAATTCAAGAAAAACTTGATGCTTTCATTGAAGCTCTTCATCAGGAGAAATAA;

The 2131-nt sequence SEQ ID NO: 27 (designed as OPA1 RTMₜₕ#114 in the Examples section):

CTAATTTAATCCACTGTTCAGTATTTAAATATTACTAATATTAAGACATATAAAGAACGAGAAC
ATTATTATAGCGTTGCTCGAGTACTAACTGGTACCTCTTCTTTTTTTTCTGCAGGTAGTAGTCGT
AGGTGATCAGAGTGCTGGAAAGACTAGTGTGTTGGAAATGATTGCCCAAGCTCGAATATTCCCAAGA
GGATCTGGGGAGATGATGACACGTTCTCCAGTTAAGGTGACTCTGAGTGAAGGTCCTCACCATGTGG
CCCTATTTAAAGATAGTTCTCGGGAGTTTGATCTTACCAAAGAAGAAGATCTTGCAGCATTAAGACA
TGAAATAGAACTTCGAATGAGGAAAAATGTGAAAGAAGGCTGTACCGTTAGCCCTGAGACCATATCC
TTAAATGTAAAAGGCCCTGGACTACAGAGGATGGTGCTTGTTGACTTACCAGGTGTGATTAATACTG
TGACATCAGGCATGGCTCCTGACACAAAGGAAACTATTTTCAGTATCAGCAAAGCTTACATGCAGAA
TCCTAATGCCATCATACTGTGTATTCAAGATGGATCTGTGGATGCTGAACGCAGTATTGTTACAGAC
TTGGTCAGTCAAATGGACCCTCATGGAAGGAGAACCATATTCGTTTTGACCAAAGTAGACCTGGCAG
AGAAAAATGTAGCCAGTCCAAGCAGGATTCAGCAGATAATTGAAGGAAAGCTCTTCCCAATGAAAGC
TTTAGGTTATTTTGCTGTTGTAACAGGAAAAGGGAACAGCTCTGAAAGCATTGAAGCTATAAGAGAA
TATGAAGAAGAGTTTTTTCAGAATTCAAAGCTCCTAAAGACAAGCATGCTAAAGGCACACCAAGTGA
CTACAAGAAATTTAAGCCTTGCAGTATCAGACTGCTTTTGGAAAATGGTACGAGAGTCTGTTGAACA
ACAGGCTGATAGTTTCAAAGCAACACGTTTTAACCTTGAAACTGAATGGAAGAATAACTATCCTCGC
CTGCGGGAACTTGACCGGAATGAACTATTTGAAAAAGCTAAAAATGAAATCCTTGATGAAGTTATCA
GTCTGAGCCAGGTTACACCAAAACATTGGGAGGAAATCCTTCAACAATCTTTGTGGGAAAGAGTATC
AACTCATGTGATTGAAAACATCTACCTTCCAGCTGCGCAGACCATGAATTCAGGAACTTTTAACACC
ACAGTGGATATCAAGCTTAAACAGTGGACTGATAAACAACTTCCTAATAAAGCAGTAGAGGTTGCTT
GGGAGACCCTACAAGAAGAATTTTCCCGCTTTATGACAGAACCGAAAGGGAAAGAGCATGATGACAT
ATTTGATAAACTTAAAGAGGCTGTTAAGGAAGAAAGTATTAAACGACACAAGTGGAATGACTTTGCG
GAGGACAGCTTGAGGGTTATTCAACACAATGCTTTGGAAGACCGATCCATATCTGATAAACAGCAAT
GGGATGCAGCTATTTATTTTATGGAAGAGGCTCTGCAGGCTCGTCTCAAGGATACTGAAAATGCAAT
TGAAAACATGGTGGGTCCAGACTGGAAAAAGAGGTGGTTATACTGGAAGAATCGGACCCAAGAACAG
TGTGTTCACAATGAAACCAAGAATGAATTGGAGAAGATGTTGAAATGTAATGAGGAGCACCCAGCTT
ATCTTGCAAGTGATGAAATAACCACAGTCCGGAAGAACCTTGAATCCCGAGGAGTAGAAGTAGATCC
AAGCTTGATTAAGGATACTTGGCATCAAGTTTATAGAAGACATTTTTTAAAAACAGCTCTAAACCAT
TGTAACCTTTGTCGAAGAGGTTTTTATTACTACCAAAGGCATTTTGTAGATTCTGAGTTGGAATGCA
ATGATGTGGTCTTGTTTTGGCGTATACAGCGCATGCTTGCTATCACCGCAAATACTTTAAGGCAACA
ACTTACAAATACTGAAGTTAGGCGATTAGAGAAAAATGTTAAAGAGGTATTGGAAGATTTTGCTGAA
GATGGTGAGAAGAAGATTAAATTGCTTACTGGTAAACGCGTTCAACTGGCGGAAGACCTCAAGAAAG
TTAGAGAAATTCAAGAAAAACTTGATGCTTTCATTGAAGCTCTTCATCAGGAGAAATAA;

The 2162-nt sequence SEQ ID NO: 28 (designed as OPA1 RTMₜₕ#128 in the Examples section):

CCTAATTTTTATTTTAAAAACTTGTGCTAATTTAAACCAGTATTTCTAAAACCAGGTTATTAAC
TTTCCATAAATCTTTTCCATAGAACGAGAACATTATTATAGCGTTGCTCGAGTACTAACTGGTA
CCTCTTCTTTTTTTTCTGCAGGTAGTAGTCGTAGGTGATCAGAGTGCTGGAAAGACTAGTGTGTTG
GAAATGATTGCCCAAGCTCGAATATTCCCAAGAGGATCTGGGGAGATGATGACACGTTCTCCAGTTA
AGGTGACTCTGAGTGAAGGTCCTCACCATGTGGCCCTATTTAAAGATAGTTCTCGGGAGTTTGATCT
TACCAAAGAAGAAGATCTTGCAGCATTAAGACATGAAATAGAACTTCGAATGAGGAAAAATGTGAAA
GAAGGCTGTACCGTTAGCCCTGAGACCATATCCTTAAATGTAAAAGGCCCTGGACTACAGAGGATGG
TGCTTGTTGACTTACCAGGTGTGATTAATACTGTGACATCAGGCATGGCTCCTGACACAAAGGAAAC
TATTTTCAGTATCAGCAAAGCTTACATGCAGAATCCTAATGCCATCATACTGTGTATTCAAGATGGA
TCTGTGGATGCTGAACGCAGTATTGTTACAGACTTGGTCAGTCAAATGGACCCTCATGGAAGGAGAA
CCATATTCGTTTTGACCAAAGTAGACCTGGCAGAGAAAAATGTAGCCAGTCCAAGCAGGATTCAGCA
GATAATTGAAGGAAAGCTCTTCCCAATGAAAGCTTTAGGTTATTTTGCTGTTGTAACAGGAAAAGGG
AACAGCTCTGAAAGCATTGAAGCTATAAGAGAATATGAAGAAGAGTTTTTTCAGAATTCAAAGCTCC
TAAAGACAAGCATGCTAAAGGCACACCAAGTGACTACAAGAAATTTAAGCCTTGCAGTATCAGACTG
CTTTTGGAAAATGGTACGAGAGTCTGTTGAACAACAGGCTGATAGTTTCAAAGCAACACGTTTTAAC
CTTGAAACTGAATGGAAGAATAACTATCCTCGCCTGCGGGAACTTGACCGGAATGAACTATTTGAAA
AAGCTAAAAATGAAATCCTTGATGAAGTTATCAGTCTGAGCCAGGTTACACCAAAACATTGGGAGGA
AATCCTTCAACAATCTTTGTGGGAAAGAGTATCAACTCATGTGATTGAAAACATCTACCTTCCAGCT
GCGCAGACCATGAATTCAGGAACTTTTAACACCACAGTGGATATCAAGCTTAAACAGTGGACTGATA
AACAACTTCCTAATAAAGCAGTAGAGGTTGCTTGGGAGACCCTACAAGAAGAATTTTCCCGCTTTAT
GACAGAACCGAAAGGGAAAGAGCATGATGACATATTTGATAAACTTAAAGAGGCTGTTAAGGAAGAA
AGTATTAAACGACACAAGTGGAATGACTTTGCGGAGGACAGCTTGAGGGTTATTCAACACAATGCTT
TGGAAGACCGATCCATATCTGATAAACAGCAATGGGATGCAGCTATTTATTTTATGGAAGAGGCTCT
GCAGGCTCGTCTCAAGGATACTGAAAATGCAATTGAAAACATGGTGGGTCCAGACTGGAAAAAGAGG
TGGTTATACTGGAAGAATCGGACCCAAGAACAGTGTGTTCACAATGAAACCAAGAATGAATTGGAGA
AGATGTTGAAATGTAATGAGGAGCACCCAGCTTATCTTGCAAGTGATGAAATAACCACAGTCCGGAA
GAACCTTGAATCCCGAGGAGTAGAAGTAGATCCAAGCTTGATTAAGGATACTTGGCATCAAGTTTAT
AGAAGACATTTTTTAAAAACAGCTCTAAACCATTGTAACCTTTGTCGAAGAGGTTTTTATTACTACC
AAAGGCATTTTGTAGATTCTGAGTTGGAATGCAATGATGTGGTCTTGTTTTGGCGTATACAGCGCAT
GCTTGCTATCACCGCAAATACTTTAAGGCAACAACTTACAAATACTGAAGTTAGGCGATTAGAGAAA
AATGTTAAAGAGGTATTGGAAGATTTTGCTGAAGATGGTGAGAAGAAGATTAAATTGCTTACTGGTA
AACGCGTTCAACTGGCGGAAGACCTCAAGAAAGTTAGAGAAATTCAAGAAAAACTTGATGCTTTCAT
TGAAGCTCTTCATCAGGAGAAATAA;

The 2181-nt acid sequence SEQ ID NO: 29 (designed as OPA1 RTMₜₕ #135 in the Examples section):

CTGCTTCTTTTTTTGCTAATTGGCAAGTTCACTATCAATTTTTCACATACCTTTATATGTCTTA
ATATTAGTAATATTTAAATACTGAACAGTGGATTAAATTAGAACGAGAACATTATTATAGCGTT
GCTCGAGTACTAACTGGTACCTCTTCTTTTTTTTCTGCAGGTAGTAGTCGTAGGTGATCAGAGTG
CTGGAAAGACTAGTGTGTTGGAAATGATTGCCCAAGCTCGAATATTCCCAAGAGGATCTGGGGAGAT
GATGACACGTTCTCCAGTTAAGGTGACTCTGAGTGAAGGTCCTCACCATGTGGCCCTATTTAAAGAT
AGTTCTCGGGAGTTTGATCTTACCAAAGAAGAAGATCTTGCAGCATTAAGACATGAAATAGAACTTC
GAATGAGGAAAAATGTGAAAGAAGGCTGTACCGTTAGCCCTGAGACCATATCCTTAAATGTAAAAGG
CCCTGGACTACAGAGGATGGTGCTTGTTGACTTACCAGGTGTGATTAATACTGTGACATCAGGCATG
GCTCCTGACACAAAGGAAACTATTTTCAGTATCAGCAAAGCTTACATGCAGAATCCTAATGCCATCA
TACTGTGTATTCAAGATGGATCTGTGGATGCTGAACGCAGTATTGTTACAGACTTGGTCAGTCAAAT
GGACCCTCATGGAAGGAGAACCATATTCGTTTTGACCAAAGTAGACCTGGCAGAGAAAAATGTAGCC
AGTCCAAGCAGGATTCAGCAGATAATTGAAGGAAAGCTCTTCCCAATGAAAGCTTTAGGTTATTTTG
CTGTTGTAACAGGAAAAGGGAACAGCTCTGAAAGCATTGAAGCTATAAGAGAATATGAAGAAGAGTT
TTTTCAGAATTCAAAGCTCCTAAAGACAAGCATGCTAAAGGCACACCAAGTGACTACAAGAAATTTA
AGCCTTGCAGTATCAGACTGCTTTTGGAAAATGGTACGAGAGTCTGTTGAACAACAGGCTGATAGTT
TCAAAGCAACACGTTTTAACCTTGAAACTGAATGGAAGAATAACTATCCTCGCCTGCGGGAACTTGA
CCGGAATGAACTATTTGAAAAAGCTAAAAATGAAATCCTTGATGAAGTTATCAGTCTGAGCCAGGTT
ACACCAAAACATTGGGAGGAAATCCTTCAACAATCTTTGTGGGAAAGAGTATCAACTCATGTGATTG
AAAACATCTACCTTCCAGCTGCGCAGACCATGAATTCAGGAACTTTTAACACCACAGTGGATATCAA
GCTTAAACAGTGGACTGATAAACAACTTCCTAATAAAGCAGTAGAGGTTGCTTGGGAGACCCTACAA
GAAGAATTTTCCCGCTTTATGACAGAACCGAAAGGGAAAGAGCATGATGACATATTTGATAAACTTA
AAGAGGCTGTTAAGGAAGAAAGTATTAAACGACACAAGTGGAATGACTTTGCGGAGGACAGCTTGAG
GGTTATTCAACACAATGCTTTGGAAGACCGATCCATATCTGATAAACAGCAATGGGATGCAGCTATT
TATTTTATGGAAGAGGCTCTGCAGGCTCGTCTCAAGGATACTGAAAATGCAATTGAAAACATGGTGG
GTCCAGACTGGAAAAAGAGGTGGTTATACTGGAAGAATCGGACCCAAGAACAGTGTGTTCACAATGA
AACCAAGAATGAATTGGAGAAGATGTTGAAATGTAATGAGGAGCACCCAGCTTATCTTGCAAGTGAT
GAAATAACCACAGTCCGGAAGAACCTTGAATCCCGAGGAGTAGAAGTAGATCCAAGCTTGATTAAGG
ATACTTGGCATCAAGTTTATAGAAGACATTTTTTAAAAACAGCTCTAAACCATTGTAACCTTTGTCG
AAGAGGTTTTTATTACTACCAAAGGCATTTTGTAGATTCTGAGTTGGAATGCAATGATGTGGTCTTG
TTTTGGCGTATACAGCGCATGCTTGCTATCACCGCAAATACTTTAAGGCAACAACTTACAAATACTG
AAGTTAGGCGATTAGAGAAAAATGTTAAAGAGGTATTGGAAGATTTTGCTGAAGATGGTGAGAAGAA
GATTAAATTGCTTACTGGTAAACGCGTTCAACTGGCGGAAGACCTCAAGAAAGTTAGAGAAATTCAA
GAAAAACTTGATGCTTTCATTGAAGCTCTTCATCAGGAGAAATAA;

The 2219-nt sequence SEQ ID NO: 30 (designed as OPA1 RTMₜₕ#162 in the Examples section):

TAATGCTGCTCTGAGTTTTTTAGTCTTTTAAAATTTCTACAGGTGAAAAAATATTTTAAAATCC
GTAAAAAAATGGTGATTGATGCATGTGTGCACATTGAAAATAGGAACAGCAAAACTATAACTGGA
TCCACTGTTTATAGAACGAGAACATTATTATAGCGTTGCTCGAGTACTAACTGGTACCTCTTCTT
TTTTTTCTGCAGGTAGTAGTCGTAGGTGATCAGAGTGCTGGAAAGACTAGTGTGTTGGAAATGATT
GCCCAAGCTCGAATATTCCCAAGAGGATCTGGGGAGATGATGACACGTTCTCCAGTTAAGGTGACTC
TGAGTGAAGGTCCTCACCATGTGGCCCTATTTAAAGATAGTTCTCGGGAGTTTGATCTTACCAAAGA
AGAAGATCTTGCAGCATTAAGACATGAAATAGAACTTCGAATGAGGAAAAATGTGAAAGAAGGCTGT
ACCGTTAGCCCTGAGACCATATCCTTAAATGTAAAAGGCCCTGGACTACAGAGGATGGTGCTTGTTG
ACTTACCAGGTGTGATTAATACTGTGACATCAGGCATGGCTCCTGACACAAAGGAAACTATTTTCAG
TATCAGCAAAGCTTACATGCAGAATCCTAATGCCATCATACTGTGTATTCAAGATGGATCTGTGGAT
GCTGAACGCAGTATTGTTACAGACTTGGTCAGTCAAATGGACCCTCATGGAAGGAGAACCATATTCG
TTTTGACCAAAGTAGACCTGGCAGAGAAAAATGTAGCCAGTCCAAGCAGGATTCAGCAGATAATTGA
AGGAAAGCTCTTCCCAATGAAAGCTTTAGGTTATTTTGCTGTTGTAACAGGAAAAGGGAACAGCTCT
GAAAGCATTGAAGCTATAAGAGAATATGAAGAAGAGTTTTTTCAGAATTCAAAGCTCCTAAAGACAA
GCATGCTAAAGGCACACCAAGTGACTACAAGAAATTTAAGCCTTGCAGTATCAGACTGCTTTTGGAA
AATGGTACGAGAGTCTGTTGAACAACAGGCTGATAGTTTCAAAGCAACACGTTTTAACCTTGAAACT
GAATGGAAGAATAACTATCCTCGCCTGCGGGAACTTGACCGGAATGAACTATTTGAAAAAGCTAAAA
ATGAAATCCTTGATGAAGTTATCAGTCTGAGCCAGGTTACACCAAAACATTGGGAGGAAATCCTTCA
ACAATCTTTGTGGGAAAGAGTATCAACTCATGTGATTGAAAACATCTACCTTCCAGCTGCGCAGACC
ATGAATTCAGGAACTTTTAACACCACAGTGGATATCAAGCTTAAACAGTGGACTGATAAACAACTTC
CTAATAAAGCAGTAGAGGTTGCTTGGGAGACCCTACAAGAAGAATTTTCCCGCTTTATGACAGAACC
GAAAGGGAAAGAGCATGATGACATATTTGATAAACTTAAAGAGGCTGTTAAGGAAGAAAGTATTAAA
CGACACAAGTGGAATGACTTTGCGGAGGACAGCTTGAGGGTTATTCAACACAATGCTTTGGAAGACC
GATCCATATCTGATAAACAGCAATGGGATGCAGCTATTTATTTTATGGAAGAGGCTCTGCAGGCTCG
TCTCAAGGATACTGAAAATGCAATTGAAAACATGGTGGGTCCAGACTGGAAAAAGAGGTGGTTATAC
TGGAAGAATCGGACCCAAGAACAGTGTGTTCACAATGAAACCAAGAATGAATTGGAGAAGATGTTGA
AATGTAATGAGGAGCACCCAGCTTATCTTGCAAGTGATGAAATAACCACAGTCCGGAAGAACCTTGA
ATCCCGAGGAGTAGAAGTAGATCCAAGCTTGATTAAGGATACTTGGCATCAAGTTTATAGAAGACAT
TTTTTAAAAACAGCTCTAAACCATTGTAACCTTTGTCGAAGAGGTTTTTATTACTACCAAAGGCATT
TTGTAGATTCTGAGTTGGAATGCAATGATGTGGTCTTGTTTTGGCGTATACAGCGCATGCTTGCTAT
CACCGCAAATACTTTAAGGCAACAACTTACAAATACTGAAGTTAGGCGATTAGAGAAAAATGTTAAA
GAGGTATTGGAAGATTTTGCTGAAGATGGTGAGAAGAAGATTAAATTGCTTACTGGTAAACGCGTTC
AACTGGCGGAAGACCTCAAGAAAGTTAGAGAAATTCAAGAAAAACTTGATGCTTTCATTGAAGCTCT
TCATCAGGAGAAATAA;

and

The 2185-nt sequence SEQ ID NO: 31 (designed as OPA1 RTMₜₕ#100 in the Examples section):

GCAATCATTTCCAACACACTAGTCTTTCCAGCACTCTGATCTCCAACCACAACAACCTTCCCAC
AAAACAAAGAACACTTATTCATATTTTTCTGATACCATCGATGTTAACGAGAACATTATTATAG
CGTTGCTCGAGTACTAACTGGTACCTCTTCTTTTTTTTCTGCAGGTAGTAGTCGTAGGTGATCA
GAGTGCTGGAAAGACTAGTGTGTTGGAAATGATTGCCCAAGCTCGAATATTCCCAAGAGGATCT
GGGGAGATGATGACACGTTCTCCAGTTAAGGTGACTCTGAGTGAAGGTCCTCACCATGTGGCCC
TATTTAAAGATAGTTCTCGGGAGTTTGATCTTACCAAAGAAGAAGATCTTGCAGCATTAAGACA
TGAAATAGAACTTCGAATGAGGAAAAATGTGAAAGAAGGCTGTACCGTTAGCCCTGAGACCATA
TCCTTAAATGTAAAAGGCCCTGGACTACAGAGGATGGTGCTTGTTGACTTACCAGGTGTGATTA
ATACTGTGACATCAGGCATGGCTCCTGACACAAAGGAAACTATTTTCAGTATCAGCAAAGCTTA
CATGCAGAATCCTAATGCCATCATACTGTGTATTCAAGATGGATCTGTGGATGCTGAACGCAGT
ATTGTTACAGACTTGGTCAGTCAAATGGACCCTCATGGAAGGAGAACCATATTCGTTTTGACCA
AAGTAGACCTGGCAGAGAAAAATGTAGCCAGTCCAAGCAGGATTCAGCAGATAATTGAAGGAAA
GCTCTTCCCAATGAAAGCTTTAGGTTATTTTGCTGTTGTAACAGGAAAAGGGAACAGCTCTGAA
AGCATTGAAGCTATAAGAGAATATGAAGAAGAGTTTTTTCAGAATTCAAAGCTCCTAAAGACAA
GCATGCTAAAGGCACACCAAGTGACTACAAGAAATTTAAGCCTTGCAGTATCAGACTGCTTTTG
GAAAATGGTACGAGAGTCTGTTGAACAACAGGCTGATAGTTTCAAAGCAACACGTTTTAACCTT
GAAACTGAATGGAAGAATAACTATCCTCGCCTGCGGGAACTTGACCGGAATGAACTATTTGAAA
AAGCTAAAAATGAAATCCTTGATGAAGTTATCAGTCTGAGCCAGGTTACACCAAAACATTGGGA
GGAAATCCTTCAACAATCTTTGTGGGAAAGAGTATCAACTCATGTGATTGAAAACATCTACCTT
CCAGCTGCGCAGACCATGAATTCAGGAACTTTTAACACCACAGTGGATATCAAGCTTAAACAGT
GGACTGATAAACAACTTCCTAATAAAGCAGTAGAGGTTGCTTGGGAGACCCTACAAGAAGAATT
TTCCCGCTTTATGACAGAACCGAAAGGGAAAGAGCATGATGACATATTTGATAAACTTAAAGAG
GCTGTTAAGGAAGAAAGTATTAAACGACACAAGTGGAATGACTTTGCGGAGGACAGCTTGAGGG
TTATTCAACACAATGCTTTGGAAGACCGATCCATATCTGATAAACAGCAATGGGATGCAGCTAT
TTATTTTATGGAAGAGGCTCTGCAGGCTCGTCTCAAGGATACTGAAAATGCAATTGAAAACATG
GTGGGTCCAGACTGGAAAAAGAGGTGGTTATACTGGAAGAATCGGACCCAAGAACAGTGTGTTC
ACAATGAAACCAAGAATGAATTGGAGAAGATGTTGAAATGTAATGAGGAGCACCCAGCTTATCT
TGCAAGTGATGAAATAACCACAGTCCGGAAGAACCTTGAATCCCGAGGAGTAGAAGTAGATCCA
AGCTTGATTAAGGATACTTGGCATCAAGTTTATAGAAGACATTTTTTAAAAACAGCTCTAAACC
ATTGTAACCTTTGTCGAAGAGGTTTTTATTACTACCAAAGGCATTTTGTAGATTCTGAGTTGGA
ATGCAATGATGTGGTCTTGTTTTGGCGTATACAGCGCATGCTTGCTATCACCGCAAATACTTTA
AGGCAACAACTTACAAATACTGAAGTTAGGCGATTAGAGAAAAATGTTAAAGAGGTATTGGAAG
ATTTTGCTGAAGATGGTGAGAAGAAGATTAAATTGCTTACTGGTAAACGCGTTCAACTGGCGGA
AGACCTCAAGAAAGTTAGAGAAATTCAAGAAAAACTTGATGCTTTCATTGAAGCTCTTCATCAG
GAGAAATAA;

The 2215-nt sequence SEQ ID NO: 32 (designed as OPA1 RTMₜₕ #130 in the Examples section):

CCAGATCCTCTTGGGAATATTCGAGCTTGGGCAATCATTTCCAACACACTAGTCTTTCCAGCAC
TCTGATCTCCAACCACAACAACCTTCCCACAAAACAAAGAACACTTATTCATATTTTTCTGATA
CCATCGATGTTAACGAGAACATTATTATAGCGTTGCTCGAGTACTAACTGGTACCTCTTCTTTT
TTTTCTGCAGGTAGTAGTCGTAGGTGATCAGAGTGCTGGAAAGACTAGTGTGTTGGAAATGATT
GCCCAAGCTCGAATATTCCCAAGAGGATCTGGGGAGATGATGACACGTTCTCCAGTTAAGGTGA
CTCTGAGTGAAGGTCCTCACCATGTGGCCCTATTTAAAGATAGTTCTCGGGAGTTTGATCTTAC
CAAAGAAGAAGATCTTGCAGCATTAAGACATGAAATAGAACTTCGAATGAGGAAAAATGTGAAA
GAAGGCTGTACCGTTAGCCCTGAGACCATATCCTTAAATGTAAAAGGCCCTGGACTACAGAGGA
TGGTGCTTGTTGACTTACCAGGTGTGATTAATACTGTGACATCAGGCATGGCTCCTGACACAAA
GGAAACTATTTTCAGTATCAGCAAAGCTTACATGCAGAATCCTAATGCCATCATACTGTGTATT
CAAGATGGATCTGTGGATGCTGAACGCAGTATTGTTACAGACTTGGTCAGTCAAATGGACCCTC
ATGGAAGGAGAACCATATTCGTTTTGACCAAAGTAGACCTGGCAGAGAAAAATGTAGCCAGTCC
AAGCAGGATTCAGCAGATAATTGAAGGAAAGCTCTTCCCAATGAAAGCTTTAGGTTATTTTGCT
GTTGTAACAGGAAAAGGGAACAGCTCTGAAAGCATTGAAGCTATAAGAGAATATGAAGAAGAGT
TTTTTCAGAATTCAAAGCTCCTAAAGACAAGCATGCTAAAGGCACACCAAGTGACTACAAGAAA
TTTAAGCCTTGCAGTATCAGACTGCTTTTGGAAAATGGTACGAGAGTCTGTTGAACAACAGGCT
GATAGTTTCAAAGCAACACGTTTTAACCTTGAAACTGAATGGAAGAATAACTATCCTCGCCTGC
GGGAACTTGACCGGAATGAACTATTTGAAAAAGCTAAAAATGAAATCCTTGATGAAGTTATCAG
TCTGAGCCAGGTTACACCAAAACATTGGGAGGAAATCCTTCAACAATCTTTGTGGGAAAGAGTA
TCAACTCATGTGATTGAAAACATCTACCTTCCAGCTGCGCAGACCATGAATTCAGGAACTTTTA
ACACCACAGTGGATATCAAGCTTAAACAGTGGACTGATAAACAACTTCCTAATAAAGCAGTAGA
GGTTGCTTGGGAGACCCTACAAGAAGAATTTTCCCGCTTTATGACAGAACCGAAAGGGAAAGAG
CATGATGACATATTTGATAAACTTAAAGAGGCTGTTAAGGAAGAAAGTATTAAACGACACAAGT
GGAATGACTTTGCGGAGGACAGCTTGAGGGTTATTCAACACAATGCTTTGGAAGACCGATCCAT
ATCTGATAAACAGCAATGGGATGCAGCTATTTATTTTATGGAAGAGGCTCTGCAGGCTCGTCTC
AAGGATACTGAAAATGCAATTGAAAACATGGTGGGTCCAGACTGGAAAAAGAGGTGGTTATACT
GGAAGAATCGGACCCAAGAACAGTGTGTTCACAATGAAACCAAGAATGAATTGGAGAAGATGTT
GAAATGTAATGAGGAGCACCCAGCTTATCTTGCAAGTGATGAAATAACCACAGTCCGGAAGAAC
CTTGAATCCCGAGGAGTAGAAGTAGATCCAAGCTTGATTAAGGATACTTGGCATCAAGTTTATA
GAAGACATTTTTTAAAAACAGCTCTAAACCATTGTAACCTTTGTCGAAGAGGTTTTTATTACTA
CCAAAGGCATTTTGTAGATTCTGAGTTGGAATGCAATGATGTGGTCTTGTTTTGGCGTATACAG
CGCATGCTTGCTATCACCGCAAATACTTTAAGGCAACAACTTACAAATACTGAAGTTAGGCGAT
TAGAGAAAAATGTTAAAGAGGTATTGGAAGATTTTGCTGAAGATGGTGAGAAGAAGATTAAATT
GCTTACTGGTAAACGCGTTCAACTGGCGGAAGACCTCAAGAAAGTTAGAGAAATTCAAGAAAAA
CTTGATGCTTTCATTGAAGCTCTTCATCAGGAGAAATAA;

The 2243-nt sequence SEQ ID NO: 33 (designed as OPA1 RTMₜₕ#158 in the Examples section):

CTTAACTGGAGAACGTGTCATCATCTCCCCAGATCCTCTTGGGAATATTCGAGCTTGGGCAATC
ATTTCCAACACACTAGTCTTTCCAGCACTCTGATCTCCAACCACAACAACCTTCCCACAAAACA
AAGAACACTTATTCATATTTTTCTGATACCATCGATGTTAACGAGAACATTATTATAGCGTTGC
TCGAGTACTAACTGGTACCTCTTCTTTTTTTTCTGCAGGTAGTAGTCGTAGGTGATCAGAGTGC
TGGAAAGACTAGTGTGTTGGAAATGATTGCCCAAGCTCGAATATTCCCAAGAGGATCTGGGGAG
ATGATGACACGTTCTCCAGTTAAGGTGACTCTGAGTGAAGGTCCTCACCATGTGGCCCTATTTA
AAGATAGTTCTCGGGAGTTTGATCTTACCAAAGAAGAAGATCTTGCAGCATTAAGACATGAAAT
AGAACTTCGAATGAGGAAAAATGTGAAAGAAGGCTGTACCGTTAGCCCTGAGACCATATCCTTA
AATGTAAAAGGCCCTGGACTACAGAGGATGGTGCTTGTTGACTTACCAGGTGTGATTAATACTG
TGACATCAGGCATGGCTCCTGACACAAAGGAAACTATTTTCAGTATCAGCAAAGCTTACATGCA
GAATCCTAATGCCATCATACTGTGTATTCAAGATGGATCTGTGGATGCTGAACGCAGTATTGTT
ACAGACTTGGTCAGTCAAATGGACCCTCATGGAAGGAGAACCATATTCGTTTTGACCAAAGTAG
ACCTGGCAGAGAAAAATGTAGCCAGTCCAAGCAGGATTCAGCAGATAATTGAAGGAAAGCTCTT
CCCAATGAAAGCTTTAGGTTATTTTGCTGTTGTAACAGGAAAAGGGAACAGCTCTGAAAGCATT
GAAGCTATAAGAGAATATGAAGAAGAGTTTTTTCAGAATTCAAAGCTCCTAAAGACAAGCATGC
TAAAGGCACACCAAGTGACTACAAGAAATTTAAGCCTTGCAGTATCAGACTGCTTTTGGAAAAT
GGTACGAGAGTCTGTTGAACAACAGGCTGATAGTTTCAAAGCAACACGTTTTAACCTTGAAACT
GAATGGAAGAATAACTATCCTCGCCTGCGGGAACTTGACCGGAATGAACTATTTGAAAAAGCTA
AAAATGAAATCCTTGATGAAGTTATCAGTCTGAGCCAGGTTACACCAAAACATTGGGAGGAAAT
CCTTCAACAATCTTTGTGGGAAAGAGTATCAACTCATGTGATTGAAAACATCTACCTTCCAGCT
GCGCAGACCATGAATTCAGGAACTTTTAACACCACAGTGGATATCAAGCTTAAACAGTGGACTG
ATAAACAACTTCCTAATAAAGCAGTAGAGGTTGCTTGGGAGACCCTACAAGAAGAATTTTCCCG
CTTTATGACAGAACCGAAAGGGAAAGAGCATGATGACATATTTGATAAACTTAAAGAGGCTGTT
AAGGAAGAAAGTATTAAACGACACAAGTGGAATGACTTTGCGGAGGACAGCTTGAGGGTTATTC
AACACAATGCTTTGGAAGACCGATCCATATCTGATAAACAGCAATGGGATGCAGCTATTTATTT
TATGGAAGAGGCTCTGCAGGCTCGTCTCAAGGATACTGAAAATGCAATTGAAAACATGGTGGGT
CCAGACTGGAAAAAGAGGTGGTTATACTGGAAGAATCGGACCCAAGAACAGTGTGTTCACAATG
AAACCAAGAATGAATTGGAGAAGATGTTGAAATGTAATGAGGAGCACCCAGCTTATCTTGCAAG
TGATGAAATAACCACAGTCCGGAAGAACCTTGAATCCCGAGGAGTAGAAGTAGATCCAAGCTTG
ATTAAGGATACTTGGCATCAAGTTTATAGAAGACATTTTTTAAAAACAGCTCTAAACCATTGTA
ACCTTTGTCGAAGAGGTTTTTATTACTACCAAAGGCATTTTGTAGATTCTGAGTTGGAATGCAA
TGATGTGGTCTTGTTTTGGCGTATACAGCGCATGCTTGCTATCACCGCAAATACTTTAAGGCAA
CAACTTACAAATACTGAAGTTAGGCGATTAGAGAAAAATGTTAAAGAGGTATTGGAAGATTTTG
CTGAAGATGGTGAGAAGAAGATTAAATTGCTTACTGGTAAACGCGTTCAACTGGCGGAAGACCT
CAAGAAAGTTAGAGAAATTCAAGAAAAACTTGATGCTTTCATTGAAGCTCTTCATCAGGAGAAA
TAA;

The 2251-nt sequence SEQ ID NO: 34 (designed as OPA1 RTMₜₕ#166 in the Examples section):

GCAATCATTTCCAACACACTAGTCTTTCCAGCACTCTGATCTCCAACCACAACAACCTTCCCAC
AAAACAAAGAACACTTATTCATATTTTTCTGATACCAAATTAAAATTAAAACCTATTTGTAATG
CTGCTCTGAGTTTTTTAGTCTTTTAAAATTTCTACAGGATCGATGTTAACGAGAACATTATTAT
AGCGTTGCTCGAGTACTAACTGGTACCTCTTCTTTTTTTTCTGCAGGTAGTAGTCGTAGGTGAT
CAGAGTGCTGGAAAGACTAGTGTGTTGGAAATGATTGCCCAAGCTCGAATATTCCCAAGAGGAT
CTGGGGAGATGATGACACGTTCTCCAGTTAAGGTGACTCTGAGTGAAGGTCCTCACCATGTGGC
CCTATTTAAAGATAGTTCTCGGGAGTTTGATCTTACCAAAGAAGAAGATCTTGCAGCATTAAGA
CATGAAATAGAACTTCGAATGAGGAAAAATGTGAAAGAAGGCTGTACCGTTAGCCCTGAGACCA
TATCCTTAAATGTAAAAGGCCCTGGACTACAGAGGATGGTGCTTGTTGACTTACCAGGTGTGAT
TAATACTGTGACATCAGGCATGGCTCCTGACACAAAGGAAACTATTTTCAGTATCAGCAAAGCT
TACATGCAGAATCCTAATGCCATCATACTGTGTATTCAAGATGGATCTGTGGATGCTGAACGCA
GTATTGTTACAGACTTGGTCAGTCAAATGGACCCTCATGGAAGGAGAACCATATTCGTTTTGAC
CAAAGTAGACCTGGCAGAGAAAAATGTAGCCAGTCCAAGCAGGATTCAGCAGATAATTGAAGGA
AAGCTCTTCCCAATGAAAGCTTTAGGTTATTTTGCTGTTGTAACAGGAAAAGGGAACAGCTCTG
AAAGCATTGAAGCTATAAGAGAATATGAAGAAGAGTTTTTTCAGAATTCAAAGCTCCTAAAGAC
AAGCATGCTAAAGGCACACCAAGTGACTACAAGAAATTTAAGCCTTGCAGTATCAGACTGCTTT
TGGAAAATGGTACGAGAGTCTGTTGAACAACAGGCTGATAGTTTCAAAGCAACACGTTTTAACC
TTGAAACTGAATGGAAGAATAACTATCCTCGCCTGCGGGAACTTGACCGGAATGAACTATTTGA
AAAAGCTAAAAATGAAATCCTTGATGAAGTTATCAGTCTGAGCCAGGTTACACCAAAACATTGG
GAGGAAATCCTTCAACAATCTTTGTGGGAAAGAGTATCAACTCATGTGATTGAAAACATCTACC
TTCCAGCTGCGCAGACCATGAATTCAGGAACTTTTAACACCACAGTGGATATCAAGCTTAAACA
GTGGACTGATAAACAACTTCCTAATAAAGCAGTAGAGGTTGCTTGGGAGACCCTACAAGAAGAA
TTTTCCCGCTTTATGACAGAACCGAAAGGGAAAGAGCATGATGACATATTTGATAAACTTAAAG
AGGCTGTTAAGGAAGAAAGTATTAAACGACACAAGTGGAATGACTTTGCGGAGGACAGCTTGAG
GGTTATTCAACACAATGCTTTGGAAGACCGATCCATATCTGATAAACAGCAATGGGATGCAGCT
ATTTATTTTATGGAAGAGGCTCTGCAGGCTCGTCTCAAGGATACTGAAAATGCAATTGAAAACA
TGGTGGGTCCAGACTGGAAAAAGAGGTGGTTATACTGGAAGAATCGGACCCAAGAACAGTGTGT
TCACAATGAAACCAAGAATGAATTGGAGAAGATGTTGAAATGTAATGAGGAGCACCCAGCTTAT
CTTGCAAGTGATGAAATAACCACAGTCCGGAAGAACCTTGAATCCCGAGGAGTAGAAGTAGATC
CAAGCTTGATTAAGGATACTTGGCATCAAGTTTATAGAAGACATTTTTTAAAAACAGCTCTAAA
CCATTGTAACCTTTGTCGAAGAGGTTTTTATTACTACCAAAGGCATTTTGTAGATTCTGAGTTG
GAATGCAATGATGTGGTCTTGTTTTGGCGTATACAGCGCATGCTTGCTATCACCGCAAATACTT
TAAGGCAACAACTTACAAATACTGAAGTTAGGCGATTAGAGAAAAATGTTAAAGAGGTATTGGA
AGATTTTGCTGAAGATGGTGAGAAGAAGATTAAATTGCTTACTGGTAAACGCGTTCAACTGGCG
GAAGACCTCAAGAAAGTTAGAGAAATTCAAGAAAAACTTGATGCTTTCATTGAAGCTCTTCATC
AGGAGAAATAA;

The 2281-nt sequence SEQ ID NO: 35 (designed as OPA1 RTMₜₕ#196 in the Examples section):

CCAGATCCTCTTGGGAATATTCGAGCTTGGGCAATCATTTCCAACACACTAGTCTTTCCAGCAC
TCTGATCTCCAACCACAACAACCTTCCCACAAAACAAAGAACACTTATTCATATTTTTCTGATA
CCAAATTAAAATTAAAACCTATTTGTAATGCTGCTCTGAGTTTTTTAGTCTTTTAAAATTTCTA
CAGGATCGATGTTAACGAGAACATTATTATAGCGTTGCTCGAGTACTAACTGGTACCTCTTCTT
TTTTTTCTGCAGGTAGTAGTCGTAGGTGATCAGAGTGCTGGAAAGACTAGTGTGTTGGAAATGA
TTGCCCAAGCTCGAATATTCCCAAGAGGATCTGGGGAGATGATGACACGTTCTCCAGTTAAGGT
GACTCTGAGTGAAGGTCCTCACCATGTGGCCCTATTTAAAGATAGTTCTCGGGAGTTTGATCTT
ACCAAAGAAGAAGATCTTGCAGCATTAAGACATGAAATAGAACTTCGAATGAGGAAAAATGTGA
AAGAAGGCTGTACCGTTAGCCCTGAGACCATATCCTTAAATGTAAAAGGCCCTGGACTACAGAG
GATGGTGCTTGTTGACTTACCAGGTGTGATTAATACTGTGACATCAGGCATGGCTCCTGACACA
AAGGAAACTATTTTCAGTATCAGCAAAGCTTACATGCAGAATCCTAATGCCATCATACTGTGTA
TTCAAGATGGATCTGTGGATGCTGAACGCAGTATTGTTACAGACTTGGTCAGTCAAATGGACCC
TCATGGAAGGAGAACCATATTCGTTTTGACCAAAGTAGACCTGGCAGAGAAAAATGTAGCCAGT
CCAAGCAGGATTCAGCAGATAATTGAAGGAAAGCTCTTCCCAATGAAAGCTTTAGGTTATTTTG
CTGTTGTAACAGGAAAAGGGAACAGCTCTGAAAGCATTGAAGCTATAAGAGAATATGAAGAAGA
GTTTTTTCAGAATTCAAAGCTCCTAAAGACAAGCATGCTAAAGGCACACCAAGTGACTACAAGA
AATTTAAGCCTTGCAGTATCAGACTGCTTTTGGAAAATGGTACGAGAGTCTGTTGAACAACAGG
CTGATAGTTTCAAAGCAACACGTTTTAACCTTGAAACTGAATGGAAGAATAACTATCCTCGCCT
GCGGGAACTTGACCGGAATGAACTATTTGAAAAAGCTAAAAATGAAATCCTTGATGAAGTTATC
AGTCTGAGCCAGGTTACACCAAAACATTGGGAGGAAATCCTTCAACAATCTTTGTGGGAAAGAG
TATCAACTCATGTGATTGAAAACATCTACCTTCCAGCTGCGCAGACCATGAATTCAGGAACTTT
TAACACCACAGTGGATATCAAGCTTAAACAGTGGACTGATAAACAACTTCCTAATAAAGCAGTA
GAGGTTGCTTGGGAGACCCTACAAGAAGAATTTTCCCGCTTTATGACAGAACCGAAAGGGAAAG
AGCATGATGACATATTTGATAAACTTAAAGAGGCTGTTAAGGAAGAAAGTATTAAACGACACAA
GTGGAATGACTTTGCGGAGGACAGCTTGAGGGTTATTCAACACAATGCTTTGGAAGACCGATCC
ATATCTGATAAACAGCAATGGGATGCAGCTATTTATTTTATGGAAGAGGCTCTGCAGGCTCGTC
TCAAGGATACTGAAAATGCAATTGAAAACATGGTGGGTCCAGACTGGAAAAAGAGGTGGTTATA
CTGGAAGAATCGGACCCAAGAACAGTGTGTTCACAATGAAACCAAGAATGAATTGGAGAAGATG
TTGAAATGTAATGAGGAGCACCCAGCTTATCTTGCAAGTGATGAAATAACCACAGTCCGGAAGA
ACCTTGAATCCCGAGGAGTAGAAGTAGATCCAAGCTTGATTAAGGATACTTGGCATCAAGTTTA
TAGAAGACATTTTTTAAAAACAGCTCTAAACCATTGTAACCTTTGTCGAAGAGGTTTTTATTAC
TACCAAAGGCATTTTGTAGATTCTGAGTTGGAATGCAATGATGTGGTCTTGTTTTGGCGTATAC
AGCGCATGCTTGCTATCACCGCAAATACTTTAAGGCAACAACTTACAAATACTGAAGTTAGGCG
ATTAGAGAAAAATGTTAAAGAGGTATTGGAAGATTTTGCTGAAGATGGTGAGAAGAAGATTAAA
TTGCTTACTGGTAAACGCGTTCAACTGGCGGAAGACCTCAAGAAAGTTAGAGAAATTCAAGAAA
AACTTGATGCTTTCATTGAAGCTCTTCATCAGGAGAAATAA;

The 2309-nt sequence SEQ ID NO: 36 (designed as OPA1 RTMₜₕ#224 in the Examples section):

CTTAACTGGAGAACGTGTCATCATCTCCCCAGATCCTCTTGGGAATATTCGAGCTTGGGCAATC
ATTTCCAACACACTAGTCTTTCCAGCACTCTGATCTCCAACCACAACAACCTTCCCACAAAACA
AAGAACACTTATTCATATTTTTCTGATACCAAATTAAAATTAAAACCTATTTGTAATGCTGCTC
TGAGTTTTTTAGTCTTTTAAAATTTCTACAGGATCGATGTTAACGAGAACATTATTATAGCGTT
GCTCGAGTACTAACTGGTACCTCTTCTTTTTTTTCTGCAGGTAGTAGTCGTAGGTGATCAGAGT
GCTGGAAAGACTAGTGTGTTGGAAATGATTGCCCAAGCTCGAATATTCCCAAGAGGATCTGGGG
AGATGATGACACGTTCTCCAGTTAAGGTGACTCTGAGTGAAGGTCCTCACCATGTGGCCCTATT
TAAAGATAGTTCTCGGGAGTTTGATCTTACCAAAGAAGAAGATCTTGCAGCATTAAGACATGAA
ATAGAACTTCGAATGAGGAAAAATGTGAAAGAAGGCTGTACCGTTAGCCCTGAGACCATATCCT
TAAATGTAAAAGGCCCTGGACTACAGAGGATGGTGCTTGTTGACTTACCAGGTGTGATTAATAC
TGTGACATCAGGCATGGCTCCTGACACAAAGGAAACTATTTTCAGTATCAGCAAAGCTTACATG
CAGAATCCTAATGCCATCATACTGTGTATTCAAGATGGATCTGTGGATGCTGAACGCAGTATTG
TTACAGACTTGGTCAGTCAAATGGACCCTCATGGAAGGAGAACCATATTCGTTTTGACCAAAGT
AGACCTGGCAGAGAAAAATGTAGCCAGTCCAAGCAGGATTCAGCAGATAATTGAAGGAAAGCTC
TTCCCAATGAAAGCTTTAGGTTATTTTGCTGTTGTAACAGGAAAAGGGAACAGCTCTGAAAGCA
TTGAAGCTATAAGAGAATATGAAGAAGAGTTTTTTCAGAATTCAAAGCTCCTAAAGACAAGCAT
GCTAAAGGCACACCAAGTGACTACAAGAAATTTAAGCCTTGCAGTATCAGACTGCTTTTGGAAA
ATGGTACGAGAGTCTGTTGAACAACAGGCTGATAGTTTCAAAGCAACACGTTTTAACCTTGAAA
CTGAATGGAAGAATAACTATCCTCGCCTGCGGGAACTTGACCGGAATGAACTATTTGAAAAAGC
TAAAAATGAAATCCTTGATGAAGTTATCAGTCTGAGCCAGGTTACACCAAAACATTGGGAGGAA
ATCCTTCAACAATCTTTGTGGGAAAGAGTATCAACTCATGTGATTGAAAACATCTACCTTCCAG
CTGCGCAGACCATGAATTCAGGAACTTTTAACACCACAGTGGATATCAAGCTTAAACAGTGGAC
TGATAAACAACTTCCTAATAAAGCAGTAGAGGTTGCTTGGGAGACCCTACAAGAAGAATTTTCC
CGCTTTATGACAGAACCGAAAGGGAAAGAGCATGATGACATATTTGATAAACTTAAAGAGGCTG
TTAAGGAAGAAAGTATTAAACGACACAAGTGGAATGACTTTGCGGAGGACAGCTTGAGGGTTAT
TCAACACAATGCTTTGGAAGACCGATCCATATCTGATAAACAGCAATGGGATGCAGCTATTTAT
TTTATGGAAGAGGCTCTGCAGGCTCGTCTCAAGGATACTGAAAATGCAATTGAAAACATGGTGG
GTCCAGACTGGAAAAAGAGGTGGTTATACTGGAAGAATCGGACCCAAGAACAGTGTGTTCACAA
TGAAACCAAGAATGAATTGGAGAAGATGTTGAAATGTAATGAGGAGCACCCAGCTTATCTTGCA
AGTGATGAAATAACCACAGTCCGGAAGAACCTTGAATCCCGAGGAGTAGAAGTAGATCCAAGCT
TGATTAAGGATACTTGGCATCAAGTTTATAGAAGACATTTTTTAAAAACAGCTCTAAACCATTG
TAACCTTTGTCGAAGAGGTTTTTATTACTACCAAAGGCATTTTGTAGATTCTGAGTTGGAATGC
AATGATGTGGTCTTGTTTTGGCGTATACAGCGCATGCTTGCTATCACCGCAAATACTTTAAGGC
AACAACTTACAAATACTGAAGTTAGGCGATTAGAGAAAAATGTTAAAGAGGTATTGGAAGATTT
TGCTGAAGATGGTGAGAAGAAGATTAAATTGCTTACTGGTAAACGCGTTCAACTGGCGGAAGAC
CTCAAGAAAGTTAGAGAAATTCAAGAAAAACTTGATGCTTTCATTGAAGCTCTTCATCAGGAGA
AATAA;

The 2294-nt sequence SEQ ID NO: 37 (designed as OPA1 RTMₜₕ#209 in the Examples section):

GCAATCATTTCCAACACACTAGTCTTTCCAGCACTCTGATCTCCAACCACAACAACCTTCCCAC
AAAACAAAGAACACTTATTCATATTTTTCTGATACCAAATTAAAATTAAAACCTATTTGTAATG
CTGCTCTGAGTTTTTTAGTCTTTTAAAATTTCTACAGGTGAAAAAATATTTTAAAATCCGTAAA
AAAATGGTGATTGATGCATCGATGTTAACGAGAACATTATTATAGCGTTGCTCGAGTACTAACT
GGTACCTCTTCTTTTTTTTCTGCAGGTAGTAGTCGTAGGTGATCAGAGTGCTGGAAAGACTAGT
GTGTTGGAAATGATTGCCCAAGCTCGAATATTCCCAAGAGGATCTGGGGAGATGATGACACGTT
CTCCAGTTAAGGTGACTCTGAGTGAAGGTCCTCACCATGTGGCCCTATTTAAAGATAGTTCTCG
GGAGTTTGATCTTACCAAAGAAGAAGATCTTGCAGCATTAAGACATGAAATAGAACTTCGAATG
AGGAAAAATGTGAAAGAAGGCTGTACCGTTAGCCCTGAGACCATATCCTTAAATGTAAAAGGCC
CTGGACTACAGAGGATGGTGCTTGTTGACTTACCAGGTGTGATTAATACTGTGACATCAGGCAT
GGCTCCTGACACAAAGGAAACTATTTTCAGTATCAGCAAAGCTTACATGCAGAATCCTAATGCC
ATCATACTGTGTATTCAAGATGGATCTGTGGATGCTGAACGCAGTATTGTTACAGACTTGGTCA
GTCAAATGGACCCTCATGGAAGGAGAACCATATTCGTTTTGACCAAAGTAGACCTGGCAGAGAA
AAATGTAGCCAGTCCAAGCAGGATTCAGCAGATAATTGAAGGAAAGCTCTTCCCAATGAAAGCT
TTAGGTTATTTTGCTGTTGTAACAGGAAAAGGGAACAGCTCTGAAAGCATTGAAGCTATAAGAG
AATATGAAGAAGAGTTTTTTCAGAATTCAAAGCTCCTAAAGACAAGCATGCTAAAGGCACACCA
AGTGACTACAAGAAATTTAAGCCTTGCAGTATCAGACTGCTTTTGGAAAATGGTACGAGAGTCT
GTTGAACAACAGGCTGATAGTTTCAAAGCAACACGTTTTAACCTTGAAACTGAATGGAAGAATA
ACTATCCTCGCCTGCGGGAACTTGACCGGAATGAACTATTTGAAAAAGCTAAAAATGAAATCCT
TGATGAAGTTATCAGTCTGAGCCAGGTTACACCAAAACATTGGGAGGAAATCCTTCAACAATCT
TTGTGGGAAAGAGTATCAACTCATGTGATTGAAAACATCTACCTTCCAGCTGCGCAGACCATGA
ATTCAGGAACTTTTAACACCACAGTGGATATCAAGCTTAAACAGTGGACTGATAAACAACTTCC
TAATAAAGCAGTAGAGGTTGCTTGGGAGACCCTACAAGAAGAATTTTCCCGCTTTATGACAGAA
CCGAAAGGGAAAGAGCATGATGACATATTTGATAAACTTAAAGAGGCTGTTAAGGAAGAAAGTA
TTAAACGACACAAGTGGAATGACTTTGCGGAGGACAGCTTGAGGGTTATTCAACACAATGCTTT
GGAAGACCGATCCATATCTGATAAACAGCAATGGGATGCAGCTATTTATTTTATGGAAGAGGCT
CTGCAGGCTCGTCTCAAGGATACTGAAAATGCAATTGAAAACATGGTGGGTCCAGACTGGAAAA
AGAGGTGGTTATACTGGAAGAATCGGACCCAAGAACAGTGTGTTCACAATGAAACCAAGAATGA
ATTGGAGAAGATGTTGAAATGTAATGAGGAGCACCCAGCTTATCTTGCAAGTGATGAAATAACC
ACAGTCCGGAAGAACCTTGAATCCCGAGGAGTAGAAGTAGATCCAAGCTTGATTAAGGATACTT
GGCATCAAGTTTATAGAAGACATTTTTTAAAAACAGCTCTAAACCATTGTAACCTTTGTCGAAG
AGGTTTTTATTACTACCAAAGGCATTTTGTAGATTCTGAGTTGGAATGCAATGATGTGGTCTTG
TTTTGGCGTATACAGCGCATGCTTGCTATCACCGCAAATACTTTAAGGCAACAACTTACAAATA
CTGAAGTTAGGCGATTAGAGAAAAATGTTAAAGAGGTATTGGAAGATTTTGCTGAAGATGGTGA
GAAGAAGATTAAATTGCTTACTGGTAAACGCGTTCAACTGGCGGAAGACCTCAAGAAAGTTAGA
GAAATTCAAGAAAAACTTGATGCTTTCATTGAAGCTCTTCATCAGGAGAAATAA;

The 2324-nt sequence SEQ ID NO: 38 (designed as OPA1 RTMₜₕ#239 in the Examples section):

CCAGATCCTCTTGGGAATATTCGAGCTTGGGCAATCATTTCCAACACACTAGTCTTTCCAGCAC
TCTGATCTCCAACCACAACAACCTTCCCACAAAACAAAGAACACTTATTCATATTTTTCTGATA
CCAAATTAAAATTAAAACCTATTTGTAATGCTGCTCTGAGTTTTTTAGTCTTTTAAAATTTCTA
CAGGTGAAAAAATATTTTAAAATCCGTAAAAAAATGGTGATTGATGCATCGATGTTAACGAGAA
CATTATTATAGCGTTGCTCGAGTACTAACTGGTACCTCTTCTTTTTTTTCTGCAGGTAGTAGTC
GTAGGTGATCAGAGTGCTGGAAAGACTAGTGTGTTGGAAATGATTGCCCAAGCTCGAATATTCC
CAAGAGGATCTGGGGAGATGATGACACGTTCTCCAGTTAAGGTGACTCTGAGTGAAGGTCCTCA
CCATGTGGCCCTATTTAAAGATAGTTCTCGGGAGTTTGATCTTACCAAAGAAGAAGATCTTGCA
GCATTAAGACATGAAATAGAACTTCGAATGAGGAAAAATGTGAAAGAAGGCTGTACCGTTAGCC
CTGAGACCATATCCTTAAATGTAAAAGGCCCTGGACTACAGAGGATGGTGCTTGTTGACTTACC
AGGTGTGATTAATACTGTGACATCAGGCATGGCTCCTGACACAAAGGAAACTATTTTCAGTATC
AGCAAAGCTTACATGCAGAATCCTAATGCCATCATACTGTGTATTCAAGATGGATCTGTGGATG
CTGAACGCAGTATTGTTACAGACTTGGTCAGTCAAATGGACCCTCATGGAAGGAGAACCATATT
CGTTTTGACCAAAGTAGACCTGGCAGAGAAAAATGTAGCCAGTCCAAGCAGGATTCAGCAGATA
ATTGAAGGAAAGCTCTTCCCAATGAAAGCTTTAGGTTATTTTGCTGTTGTAACAGGAAAAGGGA
ACAGCTCTGAAAGCATTGAAGCTATAAGAGAATATGAAGAAGAGTTTTTTCAGAATTCAAAGCT
CCTAAAGACAAGCATGCTAAAGGCACACCAAGTGACTACAAGAAATTTAAGCCTTGCAGTATCA
GACTGCTTTTGGAAAATGGTACGAGAGTCTGTTGAACAACAGGCTGATAGTTTCAAAGCAACAC
GTTTTAACCTTGAAACTGAATGGAAGAATAACTATCCTCGCCTGCGGGAACTTGACCGGAATGA
ACTATTTGAAAAAGCTAAAAATGAAATCCTTGATGAAGTTATCAGTCTGAGCCAGGTTACACCA
AAACATTGGGAGGAAATCCTTCAACAATCTTTGTGGGAAAGAGTATCAACTCATGTGATTGAAA
ACATCTACCTTCCAGCTGCGCAGACCATGAATTCAGGAACTTTTAACACCACAGTGGATATCAA
GCTTAAACAGTGGACTGATAAACAACTTCCTAATAAAGCAGTAGAGGTTGCTTGGGAGACCCTA
CAAGAAGAATTTTCCCGCTTTATGACAGAACCGAAAGGGAAAGAGCATGATGACATATTTGATA
AACTTAAAGAGGCTGTTAAGGAAGAAAGTATTAAACGACACAAGTGGAATGACTTTGCGGAGGA
CAGCTTGAGGGTTATTCAACACAATGCTTTGGAAGACCGATCCATATCTGATAAACAGCAATGG
GATGCAGCTATTTATTTTATGGAAGAGGCTCTGCAGGCTCGTCTCAAGGATACTGAAAATGCAA
TTGAAAACATGGTGGGTCCAGACTGGAAAAAGAGGTGGTTATACTGGAAGAATCGGACCCAAGA
ACAGTGTGTTCACAATGAAACCAAGAATGAATTGGAGAAGATGTTGAAATGTAATGAGGAGCAC
CCAGCTTATCTTGCAAGTGATGAAATAACCACAGTCCGGAAGAACCTTGAATCCCGAGGAGTAG
AAGTAGATCCAAGCTTGATTAAGGATACTTGGCATCAAGTTTATAGAAGACATTTTTTAAAAAC
AGCTCTAAACCATTGTAACCTTTGTCGAAGAGGTTTTTATTACTACCAAAGGCATTTTGTAGAT
TCTGAGTTGGAATGCAATGATGTGGTCTTGTTTTGGCGTATACAGCGCATGCTTGCTATCACCG
CAAATACTTTAAGGCAACAACTTACAAATACTGAAGTTAGGCGATTAGAGAAAAATGTTAAAGA
GGTATTGGAAGATTTTGCTGAAGATGGTGAGAAGAAGATTAAATTGCTTACTGGTAAACGCGTT
CAACTGGCGGAAGACCTCAAGAAAGTTAGAGAAATTCAAGAAAAACTTGATGCTTTCATTGAAG
CTCTTCATCAGGAGAAATAA;

The 2352-nt sequence SEQ ID NO: 39 (designed as OPA1 RTMₜₕ#267 in the Examples section):

CTTAACTGGAGAACGTGTCATCATCTCCCCAGATCCTCTTGGGAATATTCGAGCTTGGGCAATC
ATTTCCAACACACTAGTCTTTCCAGCACTCTGATCTCCAACCACAACAACCTTCCCACAAAACA
AAGAACACTTATTCATATTTTTCTGATACCAAATTAAAATTAAAACCTATTTGTAATGCTGCTC
TGAGTTTTTTAGTCTTTTAAAATTTCTACAGGTGAAAAAATATTTTAAAATCCGTAAAAAAATG
GTGATTGATGCATCGATGTTAACGAGAACATTATTATAGCGTTGCTCGAGTACTAACTGGTACC
TCTTCTTTTTTTTCTGCAGGTAGTAGTCGTAGGTGATCAGAGTGCTGGAAAGACTAGTGTGTTG
GAAATGATTGCCCAAGCTCGAATATTCCCAAGAGGATCTGGGGAGATGATGACACGTTCTCCAG
TTAAGGTGACTCTGAGTGAAGGTCCTCACCATGTGGCCCTATTTAAAGATAGTTCTCGGGAGTT
TGATCTTACCAAAGAAGAAGATCTTGCAGCATTAAGACATGAAATAGAACTTCGAATGAGGAAA
AATGTGAAAGAAGGCTGTACCGTTAGCCCTGAGACCATATCCTTAAATGTAAAAGGCCCTGGAC
TACAGAGGATGGTGCTTGTTGACTTACCAGGTGTGATTAATACTGTGACATCAGGCATGGCTCC
TGACACAAAGGAAACTATTTTCAGTATCAGCAAAGCTTACATGCAGAATCCTAATGCCATCATA
CTGTGTATTCAAGATGGATCTGTGGATGCTGAACGCAGTATTGTTACAGACTTGGTCAGTCAAA
TGGACCCTCATGGAAGGAGAACCATATTCGTTTTGACCAAAGTAGACCTGGCAGAGAAAAATGT
AGCCAGTCCAAGCAGGATTCAGCAGATAATTGAAGGAAAGCTCTTCCCAATGAAAGCTTTAGGT
TATTTTGCTGTTGTAACAGGAAAAGGGAACAGCTCTGAAAGCATTGAAGCTATAAGAGAATATG
AAGAAGAGTTTTTTCAGAATTCAAAGCTCCTAAAGACAAGCATGCTAAAGGCACACCAAGTGAC
TACAAGAAATTTAAGCCTTGCAGTATCAGACTGCTTTTGGAAAATGGTACGAGAGTCTGTTGAA
CAACAGGCTGATAGTTTCAAAGCAACACGTTTTAACCTTGAAACTGAATGGAAGAATAACTATC
CTCGCCTGCGGGAACTTGACCGGAATGAACTATTTGAAAAAGCTAAAAATGAAATCCTTGATGA
AGTTATCAGTCTGAGCCAGGTTACACCAAAACATTGGGAGGAAATCCTTCAACAATCTTTGTGG
GAAAGAGTATCAACTCATGTGATTGAAAACATCTACCTTCCAGCTGCGCAGACCATGAATTCAG
GAACTTTTAACACCACAGTGGATATCAAGCTTAAACAGTGGACTGATAAACAACTTCCTAATAA
AGCAGTAGAGGTTGCTTGGGAGACCCTACAAGAAGAATTTTCCCGCTTTATGACAGAACCGAAA
GGGAAAGAGCATGATGACATATTTGATAAACTTAAAGAGGCTGTTAAGGAAGAAAGTATTAAAC
GACACAAGTGGAATGACTTTGCGGAGGACAGCTTGAGGGTTATTCAACACAATGCTTTGGAAGA
CCGATCCATATCTGATAAACAGCAATGGGATGCAGCTATTTATTTTATGGAAGAGGCTCTGCAG
GCTCGTCTCAAGGATACTGAAAATGCAATTGAAAACATGGTGGGTCCAGACTGGAAAAAGAGGT
GGTTATACTGGAAGAATCGGACCCAAGAACAGTGTGTTCACAATGAAACCAAGAATGAATTGGA
GAAGATGTTGAAATGTAATGAGGAGCACCCAGCTTATCTTGCAAGTGATGAAATAACCACAGTC
CGGAAGAACCTTGAATCCCGAGGAGTAGAAGTAGATCCAAGCTTGATTAAGGATACTTGGCATC
AAGTTTATAGAAGACATTTTTTAAAAACAGCTCTAAACCATTGTAACCTTTGTCGAAGAGGTTT
TTATTACTACCAAAGGCATTTTGTAGATTCTGAGTTGGAATGCAATGATGTGGTCTTGTTTTGG
CGTATACAGCGCATGCTTGCTATCACCGCAAATACTTTAAGGCAACAACTTACAAATACTGAAG
TTAGGCGATTAGAGAAAAATGTTAAAGAGGTATTGGAAGATTTTGCTGAAGATGGTGAGAAGAA
GATTAAATTGCTTACTGGTAAACGCGTTCAACTGGCGGAAGACCTCAAGAAAGTTAGAGAAATT
CAAGAAAAACTTGATGCTTTCATTGAAGCTCTTCATCAGGAGAAATAA;

The 2302-nt sequence SEQ ID NO: 40 (designed as OPA1 RTMₜₕ#217 in the Examples section):

GCAATCATTTCCAACACACTAGTCTTTCCAGCACTCTGATCTCCA
ACCACAACAACCTTCCCACAAAACAAAGAACACTTATTCATATTT
TTCTGATACCAAATTAAAATTAAAACCTATTTGTAATGCTGCTCT
GAGTTTTTTAGTCTTTTAAAATTTCTACAGGTGAAAAAATATTTT
AAAATCCGTAAAAAAATGGTGATTGATGCATGTGTGCATCGATGT
TAACGAGAACATTATTATAGCGTTGCTCGAGTACTAACTGGTACC
TCTTCTTTTTTTTCTGCAGGTAGTAGTCGTAGGTGATCAGAGTGC
TGGAAAGACTAGTGTGTTGGAAATGATTGCCCAAGCTCGAATATT
CCCAAGAGGATCTGGGGAGATGATGACACGTTCTCCAGTTAAGGT
GACTCTGAGTGAAGGTCCTCACCATGTGGCCCTATTTAAAGATAG
TTCTCGGGAGTTTGATCTTACCAAAGAAGAAGATCTTGCAGCATT
AAGACATGAAATAGAACTTCGAATGAGGAAAAATGTGAAAGAAGG
CTGTACCGTTAGCCCTGAGACCATATCCTTAAATGTAAAAGGCCC
TGGACTACAGAGGATGGTGCTTGTTGACTTACCAGGTGTGATTAA
TACTGTGACATCAGGCATGGCTCCTGACACAAAGGAAACTATTTT
CAGTATCAGCAAAGCTTACATGCAGAATCCTAATGCCATCATACT
GTGTATTCAAGATGGATCTGTGGATGCTGAACGCAGTATTGTTAC
AGACTTGGTCAGTCAAATGGACCCTCATGGAAGGAGAACCATATT
CGTTTTGACCAAAGTAGACCTGGCAGAGAAAAATGTAGCCAGTCC
AAGCAGGATTCAGCAGATAATTGAAGGAAAGCTCTTCCCAATGAA
AGCTTTAGGTTATTTTGCTGTTGTAACAGGAAAAGGGAACAGCTC
TGAAAGCATTGAAGCTATAAGAGAATATGAAGAAGAGTTTTTTCA
GAATTCAAAGCTCCTAAAGACAAGCATGCTAAAGGCACACCAAGT
GACTACAAGAAATTTAAGCCTTGCAGTATCAGACTGCTTTTGGAA
AATGGTACGAGAGTCTGTTGAACAACAGGCTGATAGTTTCAAAGC
AACACGTTTTAACCTTGAAACTGAATGGAAGAATAACTATCCTCG
CCTGCGGGAACTTGACCGGAATGAACTATTTGAAAAAGCTAAAAA
TGAAATCCTTGATGAAGTTATCAGTCTGAGCCAGGTTACACCAAA
ACATTGGGAGGAAATCCTTCAACAATCTTTGTGGGAAAGAGTATC
AACTCATGTGATTGAAAACATCTACCTTCCAGCTGCGCAGACCAT
GAATTCAGGAACTTTTAACACCACAGTGGATATCAAGCTTAAACA
GTGGACTGATAAACAACTTCCTAATAAAGCAGTAGAGGTTGCTTG
GGAGACCCTACAAGAAGAATTTTCCCGCTTTATGACAGAACCGAA
AGGGAAAGAGCATGATGACATATTTGATAAACTTAAAGAGGCTGT
TAAGGAAGAAAGTATTAAACGACACAAGTGGAATGACTTTGCGGA
GGACAGCTTGAGGGTTATTCAACACAATGCTTTGGAAGACCGATC
CATATCTGATAAACAGCAATGGGATGCAGCTATTTATTTTATGGA
AGAGGCTCTGCAGGCTCGTCTCAAGGATACTGAAAATGCAATTGA
AAACATGGTGGGTCCAGACTGGAAAAAGAGGTGGTTATACTGGAA
GAATCGGACCCAAGAACAGTGTGTTCACAATGAAACCAAGAATGA
ATTGGAGAAGATGTTGAAATGTAATGAGGAGCACCCAGCTTATCT
TGCAAGTGATGAAATAACCACAGTCCGGAAGAACCTTGAATCCCG
AGGAGTAGAAGTAGATCCAAGCTTGATTAAGGATACTTGGCATCA
AGTTTATAGAAGACATTTTTTAAAAACAGCTCTAAACCATTGTAA
CCTTTGTCGAAGAGGTTTTTATTACTACCAAAGGCATTTTGTAGA
TTCTGAGTTGGAATGCAATGATGTGGTCTTGTTTTGGCGTATACA
GCGCATGCTTGCTATCACCGCAAATACTTTAAGGCAACAACTTAC
AAATACTGAAGTTAGGCGATTAGAGAAAAATGTTAAAGAGGTATT
GGAAGATTTTGCTGAAGATGGTGAGAAGAAGATTAAATTGCTTAC
TGGTAAACGCGTTCAACTGGCGGAAGACCTCAAGAAAGTTAGAGA
AATTCAAGAAAAACTTGATGCTTTCATTGAAGCTCTTCATCAGGA
GAAATAA;

The 2332-nt sequence SEQ ID NO: 41 (designed as OPA1 RTMₜₕ#247 in the Examples section):

CCAGATCCTCTTGGGAATATTCGAGCTTGGGCAATCATTTCCAAC
ACACTAGTCTTTCCAGCACTCTGATCTCCAACCACAACAACCTTC
CCACAAAACAAAGAACACTTATTCATATTTTTCTGATACCAAATT
AAAATTAAAACCTATTTGTAATGCTGCTCTGAGTTTTTTAGTCTT
TTAAAATTTCTACAGGTGAAAAAATATTTTAAAATCCGTAAAAAA
ATGGTGATTGATGCATGTGTGCATCGATGTTAACGAGAACATTAT
TATAGCGTTGCTCGAGTACTAACTGGTACCTCTTCTTTTTTTTCT
GCAGGTAGTAGTCGTAGGTGATCAGAGTGCTGGAAAGACTAGTGT
GTTGGAAATGATTGCCCAAGCTCGAATATTCCCAAGAGGATCTGG
GGAGATGATGACACGTTCTCCAGTTAAGGTGACTCTGAGTGAAGG
TCCTCACCATGTGGCCCTATTTAAAGATAGTTCTCGGGAGTTTGA
TCTTACCAAAGAAGAAGATCTTGCAGCATTAAGACATGAAATAGA
ACTTCGAATGAGGAAAAATGTGAAAGAAGGCTGTACCGTTAGCCC
TGAGACCATATCCTTAAATGTAAAAGGCCCTGGACTACAGAGGAT
GGTGCTTGTTGACTTACCAGGTGTGATTAATACTGTGACATCAGG
CATGGCTCCTGACACAAAGGAAACTATTTTCAGTATCAGCAAAGC
TTACATGCAGAATCCTAATGCCATCATACTGTGTATTCAAGATGG
ATCTGTGGATGCTGAACGCAGTATTGTTACAGACTTGGTCAGTCA
AATGGACCCTCATGGAAGGAGAACCATATTCGTTTTGACCAAAGT
AGACCTGGCAGAGAAAAATGTAGCCAGTCCAAGCAGGATTCAGCA
GATAATTGAAGGAAAGCTCTTCCCAATGAAAGCTTTAGGTTATTT
TGCTGTTGTAACAGGAAAAGGGAACAGCTCTGAAAGCATTGAAGC
TATAAGAGAATATGAAGAAGAGTTTTTTCAGAATTCAAAGCTCCT
AAAGACAAGCATGCTAAAGGCACACCAAGTGACTACAAGAAATTT
AAGCCTTGCAGTATCAGACTGCTTTTGGAAAATGGTACGAGAGTC
TGTTGAACAACAGGCTGATAGTTTCAAAGCAACACGTTTTAACCT
TGAAACTGAATGGAAGAATAACTATCCTCGCCTGCGGGAACTTGA
CCGGAATGAACTATTTGAAAAAGCTAAAAATGAAATCCTTGATGA
AGTTATCAGTCTGAGCCAGGTTACACCAAAACATTGGGAGGAAAT
CCTTCAACAATCTTTGTGGGAAAGAGTATCAACTCATGTGATTGA
AAACATCTACCTTCCAGCTGCGCAGACCATGAATTCAGGAACTTT
TAACACCACAGTGGATATCAAGCTTAAACAGTGGACTGATAAACA
ACTTCCTAATAAAGCAGTAGAGGTTGCTTGGGAGACCCTACAAGA
AGAATTTTCCCGCTTTATGACAGAACCGAAAGGGAAAGAGCATGA
TGACATATTTGATAAACTTAAAGAGGCTGTTAAGGAAGAAAGTAT
TAAACGACACAAGTGGAATGACTTTGCGGAGGACAGCTTGAGGGT
TATTCAACACAATGCTTTGGAAGACCGATCCATATCTGATAAACA
GCAATGGGATGCAGCTATTTATTTTATGGAAGAGGCTCTGCAGGC
TCGTCTCAAGGATACTGAAAATGCAATTGAAAACATGGTGGGTCC
AGACTGGAAAAAGAGGTGGTTATACTGGAAGAATCGGACCCAAGA
ACAGTGTGTTCACAATGAAACCAAGAATGAATTGGAGAAGATGTT
GAAATGTAATGAGGAGCACCCAGCTTATCTTGCAAGTGATGAAAT
AACCACAGTCCGGAAGAACCTTGAATCCCGAGGAGTAGAAGTAGA
TCCAAGCTTGATTAAGGATACTTGGCATCAAGTTTATAGAAGACA
TTTTTTAAAAACAGCTCTAAACCATTGTAACCTTTGTCGAAGAGG
TTTTTATTACTACCAAAGGCATTTTGTAGATTCTGAGTTGGAATG
CAATGATGTGGTCTTGTTTTGGCGTATACAGCGCATGCTTGCTAT
CACCGCAAATACTTTAAGGCAACAACTTACAAATACTGAAGTTAG
GCGATTAGAGAAAAATGTTAAAGAGGTATTGGAAGATTTTGCTGA
AGATGGTGAGAAGAAGATTAAATTGCTTACTGGTAAACGCGTTCA
ACTGGCGGAAGACCTCAAGAAAGTTAGAGAAATTCAAGAAAAACT
TGATGCTTTCATTGAAGCTCTTCATCAGGAGAAATAA;

and
The 2360-nt sequence SEQ ID NO: 42 (designed as OPA1 RTMₜₕ#275 in the Examples section):

CTTAACTGGAGAACGTGTCATCATCTCCCCAGATCCTCTTGGGAA
TATTCGAGCTTGGGCAATCATTTCCAACACACTAGTCTTTCCAGC
ACTCTGATCTCCAACCACAACAACCTTCCCACAAAACAAAGAACA
CTTATTCATATTTTTCTGATACCAAATTAAAATTAAAACCTATTT
GTAATGCTGCTCTGAGTTTTTTAGTCTTTTAAAATTTCTACAGGT
GAAAAAATATTTTAAAATCCGTAAAAAAATGGTGATTGATGCATG
TGTGCATCGATGTTAACGAGAACATTATTATAGCGTTGCTCGAGT
ACTAACTGGTACCTCTTCTTTTTTTTCTGCAGGTAGTAGTCGTAG
GTGATCAGAGTGCTGGAAAGACTAGTGTGTTGGAAATGATTGCCC
AAGCTCGAATATTCCCAAGAGGATCTGGGGAGATGATGACACGTT
CTCCAGTTAAGGTGACTCTGAGTGAAGGTCCTCACCATGTGGCCC
TATTTAAAGATAGTTCTCGGGAGTTTGATCTTACCAAAGAAGAAG
ATCTTGCAGCATTAAGACATGAAATAGAACTTCGAATGAGGAAAA
ATGTGAAAGAAGGCTGTACCGTTAGCCCTGAGACCATATCCTTAA
ATGTAAAAGGCCCTGGACTACAGAGGATGGTGCTTGTTGACTTAC
CAGGTGTGATTAATACTGTGACATCAGGCATGGCTCCTGACACAA
AGGAAACTATTTTCAGTATCAGCAAAGCTTACATGCAGAATCCTA
ATGCCATCATACTGTGTATTCAAGATGGATCTGTGGATGCTGAAC
GCAGTATTGTTACAGACTTGGTCAGTCAAATGGACCCTCATGGAA
GGAGAACCATATTCGTTTTGACCAAAGTAGACCTGGCAGAGAAAA
ATGTAGCCAGTCCAAGCAGGATTCAGCAGATAATTGAAGGAAAGC
TCTTCCCAATGAAAGCTTTAGGTTATTTTGCTGTTGTAACAGGAA
AAGGGAACAGCTCTGAAAGCATTGAAGCTATAAGAGAATATGAAG
AAGAGTTTTTTCAGAATTCAAAGCTCCTAAAGACAAGCATGCTAA
AGGCACACCAAGTGACTACAAGAAATTTAAGCCTTGCAGTATCAG
ACTGCTTTTGGAAAATGGTACGAGAGTCTGTTGAACAACAGGCTG
ATAGTTTCAAAGCAACACGTTTTAACCTTGAAACTGAATGGAAGA
ATAACTATCCTCGCCTGCGGGAACTTGACCGGAATGAACTATTTG
AAAAAGCTAAAAATGAAATCCTTGATGAAGTTATCAGTCTGAGCC
AGGTTACACCAAAACATTGGGAGGAAATCCTTCAACAATCTTTGT
GGGAAAGAGTATCAACTCATGTGATTGAAAACATCTACCTTCCAG
CTGCGCAGACCATGAATTCAGGAACTTTTAACACCACAGTGGATA
TCAAGCTTAAACAGTGGACTGATAAACAACTTCCTAATAAAGCAG
TAGAGGTTGCTTGGGAGACCCTACAAGAAGAATTTTCCCGCTTTA
TGACAGAACCGAAAGGGAAAGAGCATGATGACATATTTGATAAAC
TTAAAGAGGCTGTTAAGGAAGAAAGTATTAAACGACACAAGTGGA
ATGACTTTGCGGAGGACAGCTTGAGGGTTATTCAACACAATGCTT
TGGAAGACCGATCCATATCTGATAAACAGCAATGGGATGCAGCTA
TTTATTTTATGGAAGAGGCTCTGCAGGCTCGTCTCAAGGATACTG
AAAATGCAATTGAAAACATGGTGGGTCCAGACTGGAAAAAGAGGT
GGTTATACTGGAAGAATCGGACCCAAGAACAGTGTGTTCACAATG
AAACCAAGAATGAATTGGAGAAGATGTTGAAATGTAATGAGGAGC
ACCCAGCTTATCTTGCAAGTGATGAAATAACCACAGTCCGGAAGA
ACCTTGAATCCCGAGGAGTAGAAGTAGATCCAAGCTTGATTAAGG
ATACTTGGCATCAAGTTTATAGAAGACATTTTTTAAAAACAGCTC
TAAACCATTGTAACCTTTGTCGAAGAGGTTTTTATTACTACCAAA
GGCATTTTGTAGATTCTGAGTTGGAATGCAATGATGTGGTCTTGT
TTTGGCGTATACAGCGCATGCTTGCTATCACCGCAAATACTTTAA
GGCAACAACTTACAAATACTGAAGTTAGGCGATTAGAGAAAAATG
TTAAAGAGGTATTGGAAGATTTTGCTGAAGATGGTGAGAAGAAGA
TTAAATTGCTTACTGGTAAACGCGTTCAACTGGCGGAAGACCTCA
AGAAAGTTAGAGAAATTCAAGAAAAACTTGATGCTTTCATTGAAG
CTCTTCATCAGGAGAAATAA.

### II-Recombinant Vectors and Cells Comprising Nucleic Acid OPA1 Trans-Splicing Molecules

The present invention also provides a recombinant vector comprising a nucleic acid OPA1 pre-mRNA trans-splicing molecule described herein. More particular, a nucleic acid OPA1 pre-mRNA trans-splicing molecule according to the invention may be recombinantly engineered into a variety of host vector systems that also provide for replication of the DNA in large scale and contain the necessary elements for directing the transcription of the pre-mRNA trans-splicing molecule. The use of such a construct to transduce target cells in the patient will result in the transcription of sufficient amounts of the pre-mRNA trans-splicing molecule that will form complementary bases pairing with the endogenously expressed OPA1 pre-mRNA targets and thereby facilitate the pre-mRNA trans-splicing reaction. Such a vector remains episomal, as long as it can be transcribed to produce the desired RNA. Methods for assembly of recombinant vectors, which generally include culturing a host cell, are known in the art. See, e.g., Ausubel et al., Current Protocols in Molecular Biology, John Wiley & Sons, New York, 1989; Kay, M. A. et al., Nat. Medic, 2001, 7 (I): 33-40; and Walther W. and Stein U., Drugs 2000, 60 (2): 249-71.

Vectors comprising a nucleic acid OPA1 pre-mRNA trans-splicing molecule of interest can be plasmid, viral, or others known in the art, used for replication and expression in mammalian target cells. Expression of the pre-mRNA trans-splicing molecule can be regulated by any promoter/enhancer sequences known in the art to act in mammalian, preferably human cells. Such promoters/enhancers can be inducible or constitutive. Such promoters include, but are not limited to the SV40 early promoter region, the promoter contained in the 3′ long terminal repeat of the Rous sarcoma virus, the herpes thymidine kinase promoter, the regulatory sequences of the metallothionein gene, the viral cytomegalovirus CMV promoter, the human chorionic gonadotropin—P promoter, the CAG promoter (also known as CBA promoter or CAGGS promoter-CMV enhancer, chicken beta-actin promoter and rabbit beta-globin splice acceptor site), the human elongation factor-1 alpha (EF-1α) promoter, the ubiquitin UBC promoter, the phosphoglycerate kinase PGK promoter, the OPA1 promoter, and the like. In certain embodiments, promoters are able to give rise to gene expression in retinal cells, for example in RPE cells (e.g., RPE65, VMD2 or OA1 promoters), in photoreceptors (e.g., rhodopsin kinase (RHO), rhodopsin (RHO), or opsin (OP) human promoters) or preferably in retinal ganglion cells (RGCs) (e.g., human connexin36 (cx36) or SNCG promoters). According to certain preferred embodiments, the promoter is SNCG promoter (see US Publication No. 2018/0355354).

Any type of cloning vectors, including plasmids, minicircles, cosmids, YAC vectors, BAC vectors and viral vectors, can be used to prepare the recombinant DNA construct which can be introduced directly into the tissue site. Alternatively, viral vectors can be used which selectively infect the desired target cell according to the selected serotype and pseudotyping. Vectors for use in the practice of the invention include any eukaryotic expression vectors, including but not limited to viral expression vectors such as those derived from the class of retroviruses, Modified Vaccinia Virus Ankara, adenoviruses or adeno-associated viruses.

In certain preferred embodiments, the recombinant vector is an Adeno-Associated Virus (AAV) or a recombinant Adeno-Associated Virus (rAAV) or a single-stranded Adeno-Associated Virus (ssAAV) or a self-complementary Adeno-Associated Virus (scAAV). The use of AAVs or rAAVs is a common mode of exogenous delivery of DNA as it is relatively non-toxic, provides efficient gene transfer, and can be easily optimized for specific purposes. In certain embodiments, the vector is a rAAV carrying the pre-mRNA trans-splicing molecule and driven by a promoter that expresses a pre-mRNA trans-splicing molecule in selected cells of a subject, e.g., ocular cells such as retinal ganglion cells (RGCs) or cells of the auditory nerve such as spiral ganglion neurons (SGNs), and the like. More than 30 naturally occurring serotypes of AAV are available. Many natural variants in the AAV capsid exist, allowing identification and use of an AAV with properties specifically suited for target cells. AAV viruses may be engineered by conventional molecular biology techniques, making it possible to optimize these particles for cell specific delivery of the pre-mRNA trans-splicing molecule nucleic acid sequences, for minimizing immunogenicity, for tuning stability and particle lifetime, for efficient degradation, for accurate delivery to the nucleus, etc.

In certain embodiments, a pre-mRNA trans-splicing molecule according to the present invention is delivered to a target cell in vitro (or ex vivo). Various delivery systems are known and can be used to transfer a pre-mRNA trans-splicing molecule of the invention into cells, e.g., encapsulation in liposomes, microparticles, microcapsules, receptor-mediated endocytosis, construction of a nucleic acid as part of a retroviral, adenoviral, adeno-associated viruses or other vector, injection of DNA, electroporation, calcium phosphate mediated transfection, pure high-lipid based versus mixed lipid and non-lipid based transfection reagents (for a review, see Chong et al., PeerJ, 2021, 21, 9: e11165), etc. In this case, the nucleic acid OPA1 pre-mRNA trans-splicing molecule, which may be in any form used by one skilled in the art, binds to a pre-mRNA and mediates a trans-splicing reaction resulting in the formation of a chimeric mRNA comprising a portion of the exogenous and therapeutic nucleic acid pre-mRNA OPA1 trans-splicing molecule spliced to a portion of the endogenous OPA1 pre-mRNA. Thus, the present invention also relates to a cell comprising a nucleic acid OPA1 pre-mRNA trans-splicing molecule described herein or a recombinant vector comprising a nucleic acid OPA1 pre-mRNA trans-splicing molecule described herein.

In certain embodiments, the cell is a target cell. As used herein, the term “target cell” refers to an ocular cell, which is any cell associated with the function of the eye and expressing the OPA1 gene. Such target cells include retinal ganglion cells (RGCs), photoreceptor cells, amacrine cells, horizontal cells, and bipolar cells. Preferentially, the target cells are RGCs. The target cell may also be cells of the auditory nerve such as SGNs. The OPA1 gene is expressed in the eye as well as in other organs, including in the brain, heart, skeletal muscle, liver and testis. The term “target cells” may also include these extra-ocular or auditory nerve cells.

In certain embodiments, the cell is a host cell. As used herein, the term “host cell” refers to a packaging cell line in which the recombinant AAV is produced from a plasmid.

III-Therapeutic Uses of Nucleic Acid OPA1 pre-mRNA Trans-Splicing Molecule et Methods of Treatment

The present invention relates to a nucleic acid OPA1 pre-mRNA trans-splicing molecule according to the present invention, or a recombinant vector comprising the pre-mRNA trans-splicing molecule, or a cell comprising the pre-mRNA trans-splicing molecule or the recombinant vector (optionally after formulation with one or more appropriate pharmaceutically acceptable carriers or excipients (see below)) for use to correct a pathogenic mutation in the OPA1 gene of a subject. Thus, the compounds and compositions of the present invention are useful in methods of treating diseases or disorders caused by a mutation in the OPA1 gene. Such diseases and disorders include, in particular, Autosomal Dominant Optic Atrophy (ADOA), Autosomal Dominant Optic Atrophy Plus (ADOA+) and Behr's Syndrome. Other diseases and disorders include susceptibility to normal tension glaucoma (OMIM #606657), which is a progressive optic neuropathy associated with glaucomatous optic nerve damage and visual field loss where the intraocular pressure (IOP) is measured consistently below 21 mm Hg. Extra-ophthalmic diseases and disorders linked to mutations in the OPA1 gene include sensorineural deafness, peripheral neuropathies, Parkinson syndrome, mitochondrial myopathy, and heart disease. In certain embodiments, the mitochondrial myopathy is mitochondrial depletion syndrome 14 (OMIN #616895), which is characterized by severe lethal infantile mitochondrial encephalomyopathy and hypertrophic cardiomyopathy, with hypotonia and peripheral hypertonia with opisthotonic posturing, as well as feeding difficulties and profound neurodevelopmental delay.

A therapeutic method according to the present invention involves contacting a target OPA1 gene product (e.g., OPA1 pre-mRNA) with an OPA1 pre-mRNA trans-splicing molecule according to the present invention under conditions in which a coding domain of the pre-mRNA trans-splicing molecule is spliced to the target OPA1 gene product to replace a part of the targeted gene product carrying one or more mutations, with a functional (i.e., healthy), or normal or wild-type or corrected or optimized mRNA of the targeted gene, in order to correct expression and functions of OPA1 protein isoforms in the target cell.

In certain embodiments, the contacting involves direct administration to the affected subject. In other embodiments, the contacting occurs ex vivo to the cultured cell and following repair, the ex vivo-treated cell is reimplanted in the subject from whom the cell was extracted.

In certain embodiments, administration of a therapeutically effective amount of a compound or composition according to the present invention to an individual suffering from a disease or disorder associated with one or more OPA1 gene mutations and/or biallelic mutations in the OPA1 gene, alleviates one or more symptoms selected from the group consisting of: loss of visual acuity, loss of central vision, impairment of color vision, centrocecal scotomas, temporal pallor of the optic disc, circumpapillary telangiectatic microangiopathy, swelling of the retinal nerve fiber layer around the disc (pseudoedema), or optic atrophy. In certain embodiments, administration of a therapeutically effective amount of a compound or composition according to the present invention is able to stop progression of one or more of such symptoms. In certain embodiments, the treatment stops progression of visual acuity loss. In another embodiments, the treatment stops progression of color vision disturbances and/or loss. In certain embodiments, the treatment improves the visual acuity from below 20/400, or from below about 20/400, to 20/100, to about 20/100, or to better than 20/100. In other embodiments, the treatment improves color vision.

In certain embodiments, administration of a therapeutically effective amount of a compound or composition according to the present invention to an individual suffering from syndromic disease or disorder associated with an OPA1 gene mutation (e.g., a subject suffering from ADOA+ or from Behr's syndrome) alleviates one or more extra-ocular manifestations such as chronic progressive external ophthalmoplegia, proximal myopathy, ataxia and axonal sensory motor polyneuropathy, spinocerebellar degeneration resulting in ataxia, pyramidal signs, peripheral neuropathy and developmental delay.

### 1. Indications

In many embodiments, the subject is a human being, and more specifically is a patient who has been diagnosed with a hereditary optic neuropathy associated with a mutation in the OPA1 gene, as listed above. In certain embodiments, the subject is known to be at risk of developing such a hereditary optic neuropathy.

The subject may be of any age during which treatment or prophylactic therapy may be beneficial. For example, in some embodiments, the subject is 0-5 years of age, 5-10 years of age, 10-20 years of age, 20-30 years of age, 30-50 years of age, 50-70 years of age, or more than 70 years of age. In certain embodiments, the subject is 12 months of age or older, 18 months of age or older, 2 years of age or older, 3 years of age or older, 4 years of age or older, 5 years of age or older, 6 years of age or older, 7 years of age or older, 8 years of age or older, 9 years of age or older, or 10 years of age or older.

### 2. Administration

A nucleic acid OPA1 pre-mRNA trans-splicing molecule according to the present invention, or a recombinant vector comprising the nucleic acid OPA1 pre-mRNA trans-splicing molecule, or an ex vivo-treated cell, optionally after formulation into a pharmaceutical composition, can be administered to a subject in need thereof by any suitable route. Suitable methods of administration include, but are not limited to, subretinal injection, intravitreal injection, or intravenous injection, intrathecal injection. Injection via the palpebral vein or any ophthalmic veins to ocular cells may also be used. Still other modes of administration may be selected by one skilled in the art.

### 3. Dosage

Administration of a nucleic acid OPA1 pre-mRNA trans-splicing molecule according to the present invention, or of a recombinant vector comprising the nucleic acid OPA1 pre-mRNA trans-splicing molecule, or of a cell comprising the nucleic acid OPA1 pre-mRNA trans-splicing molecule or the recombinant vector, (optionally after formulation with one or more appropriate pharmaceutically acceptable carriers or excipients) will be in a dosage such that the amount delivered is effective for the intended purpose. The dose administered will depend upon the desired therapeutic effect, the degree to which the disorder has developed, the age, sex, weight and general health condition of the patient, and the targeted cell, as well as upon the use (or not) of concomitant therapies, and other clinical factors. These factors are readily determinable by the attending physician in the course of the therapy. Alternatively, or additionally, the dosage to be administered can be determined from studies using animal models. Adjusting the dose to achieve maximal efficacy based on these or other methods are well known in the art and are within the capabilities of trained physicians. As studies are conducted using compounds of the present invention, further information will emerge regarding the appropriate dosage levels.

A treatment according to the present invention may consist of a single dose or multiple doses (e.g., multiple administrations over some period, e.g., throughout several years of the life of a patient). Exemplary courses of treatment may comprise weekly, biweekly, monthly, quarterly, biannually, or yearly treatments. Alternatively, treatment may proceed in phases whereby multiple doses are administered initially, and subsequently fewer and less frequent doses are needed.

For example, in embodiments where the therapeutic agent is a recombinant adeno-associated virus carrying a nucleic acid OPA1 trans-splicing molecule according to the present invention, it is desirable that the lowest effective dosage (total genome copies delivered) of virus be utilized in order to reduce the risk of undesirable effects, such as toxicity, and other issues related to administration to the eye, e.g., retinal dysplasia and detachment. In certain embodiments, an effective dosage ranges between about 10⁸ and 10¹³ vector genomes (vg) per dose. The effective dosage may be comprised between 10⁹ and 10¹³ vg. For example, the effective dosage may be about 1.5×10¹¹ vg, or about 1.5×10¹⁰ vg, or about 2.8×10¹¹ vg, or about 5×10¹¹ vg, or about 1.5×10¹² vg, or yet about 1.5×10¹³ vg. Still other dosages in these ranges or in other units may be selected by the attending physician, taking into account the factors mentioned above.

The dose may be delivered in a volume of from about 50 μL to about 1 mL, including all numbers within the range, depending on the size of the area to be treated, the viral titer used, the route of administration and the desired effect of the method. In particular, the volume may be about 50 μL, about 75 μL, about 100 μL, about 125 μL, about 150 μL, about 175 μL, about 200 μL, about 250 μL, about 300 μL, about 350 μL, about 400 μL, about 450 μL, about 500 μL, about 600 μL, about 700 μL, about 800 μL, about 900 μL or about 1,000 μL.

Alternatively, or additionally, the volume and/or concentration of a recombinant AAV composition according to the present invention are selected so that only certain anatomical regions having target cells are impacted. In other embodiments, the volume and/or concentration of a recombinant AAV composition according to the present invention are selected in order to reach larger portions of the eye.

### 4. Concomitant Therapies

A therapeutic method according to the present invention may be performed in combination with another, or secondary therapy. The therapy may be any now known, or as yet unknown, therapy which helps prevent, arrest or ameliorate any of the effects associated with a pathogenic OPA1 mutation. The secondary therapy is selected such that it does not interfere with OPA1 pre-mRNA binding domain or its targeted site unless it can favor the interaction and the trans-splicing reaction between the endogenous mutated targeted pre-mRNA and the exogenous corrective pre-mRNA trans-splicing molecule, as scaffolding agent. The secondary therapy can be administered before, concurrent with, or after administration of a therapeutic method described herein. In one embodiment, a secondary therapy involves non-specific approaches for maintaining the health of the ocular cells, in particular retinal ganglion cells (RGCs), such as administration of neurotrophic factors, antioxidants, anti-apoptotic agents.

### 5. Monitoring of Therapeutic Efficacy of a Treatment

The effects of a treatment according to the present invention may be assessed using any suitable method known in the art, for example by performing functional and/or imaging studies. These studies include, but are not limited to, electroretinography (ERG), perimetry, topographical mapping of the layers of the retina and measurement of the thickness of its layers by means of confocal scanning laser ophthalmoscopy (cSLO) and full field optical coherence tomography (OCT), optical coherence tomography angiography (OCTA), tomographical mapping of cone density via adaptive optics (OA). In addition, visual field studies, perimetry and microperimetry, mobility testing, visual acuity, color vision testing may be performed. The results of imaging and/or functional studies may be used to modify, adapt, or optimize the treatment.

### IV-Pharmaceutical Compositions and Kits

### 1. Pharmaceutical Compositions

The present invention provides pharmaceutical compositions including a nucleic acid OPA1 pre-mRNA trans-splicing molecule described herein, or a recombinant vector comprising such trans-splicing molecule, or a cell comprising the trans-splicing molecule or the recombinant vector, and a pharmaceutically acceptable carrier or excipient. Preferable, a pharmaceutical composition comprises an effective amount of the nucleic acid OPA1 pre-mRNA trans-splicing molecule, recombinant vector, or cells. In some embodiments, a pharmaceutical composition further comprises one or more additional biologically active agents.

The pharmaceutical compositions of the present invention may be formulated in dosage unit form for ease of administration and uniformity of dosage. The expression “unit dosage form”, as used herein, refers to a physically discrete unit of the nucleic acid OPA1 trans-splicing molecule, or the recombinant vector, or the cell for the patient to be treated. It will be understood, however, that the total dosage of the compositions will be decided by the attending physician, pharmacist or biologist within the scope of sound medical judgement.

a. Formulation. A pharmaceutical composition described herein may be administered using any route of administration effective for achieving the desired therapeutic effect. The optimal pharmaceutical formulation can be varied depending upon the route of administration and desired dosage.

A pharmaceutical composition according to the present invention can be in the form of an injectable solution. Injectable preparations, for example, sterile injectable aqueous or oleaginous suspensions may be formulated according to the known art using suitable dispersing or wetting agents, and suspending agents. The sterile injectable preparation may also be a sterile injectable solution, suspension or emulsion in a non-toxic diluent or solvent, such as mannitol, 1,3-butanediol, water (e.g., sterile, pyrogen-free water), Ringer's solution, isotonic sodium chloride solution, sterile, pyrogen-free phosphate buffered saline other buffers, e.g., HEPES, to maintain pH at appropriate physiological levels, and the like. In addition, sterile, fixed oils are conventionally employed as a solution or suspending medium. For this purpose, any bland fixed oil can be employed including synthetic mono- or di-glycerides. Fatty acids such as oleic acid may also be used in the preparation of injectable formulations.

In certain embodiments, a pharmaceutical composition according to the present invention comprises a surfactant. Appropriate surfactants such as PBS-Pluronic F-68 (Poloxamer 188, also known as LUTROL®F68), may be included as they are known to prevent AAV from sticking to inert surfaces and thus ensure delivery of the desired dose. Other appropriate surfactants include TWEEN®. For example, if the pharmaceutical composition is to be stored long-term, it may be frozen in the presence of TWEEN® or in the presence of glycerol.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

For injectable formulations, the pharmaceutical compositions can be in lyophilized power in admixture with suitable excipients in a suitable vial or tube. Before use in the clinic, the drugs may be reconstituted by dissolving the lyophilized powder in a suitable solvent system to form a composition suitable for injection (e.g., subretinal, intravitreal or subconjunctival injection).

It is also envisaged that the pharmaceutical composition of the present invention is formulated/administered as eye drops.

Materials and methods for producing various formulations are known in the art and may be adapted for practicing the subject invention. Suitable formulations for the delivery of DNA molecules, recombinants vectors or cells can be found, for example, in “Remington's Pharmaceutical Sciences”, E. W. Martin, 19th Ed., 1995, Mack Publishing Co.: Easton, PA; Liberman and Lachman, Eds., “Pharmaceutical Dosage Forms”, Marcel Decker, New York, N.Y., 1980; and “Pharmaceutical Dosage Forms and Drug Delivery Systems”, 17th Ed., Lippincott Williams & Wilkins, 1999.

Ex vivo-treated cells may be formulated with a pharmaceutical carrier for administration in any convenient way for use in medicine, for example with a pharmaceutically acceptable ophthalmic formulation for intraocular injection. A pharmaceutical composition comprising ex vivo-treated cells used in a method according to the present invention may be transplanted in a suspension, gel, colloid, slurry, or mixture. The preparation may desirably be encapsulated or injected in a viscous form into the vitreous humor for delivery to the site of retinal damage. Also, at the time of injection, cryopreserved ex vivo-treated cells may be resuspended with commercially available balanced salt solution to achieve the desired osmolality and concentration for administration by subretinal injection. When administering the composition by intravitreal injection, for example, the solution may be concentrated so that minimized volumes may be delivered. Concentrations for injections may be at any amount that is effective and non-toxic. The pharmaceutical compositions of ex vivo-treated cells for treatment of a patient may be formulated at doses of at least about 10⁴ cells/mL, for example at doses of at least about 10³, 10⁴, 10⁵, 10⁶, 10⁷, 10⁸, 10⁹, or 10¹⁰ cells/mL.

b. Additional Biologically Active Agents. In certain embodiments, the nucleic acid OPA1 pre-mRNA trans-splicing molecule, or the recombinant vector comprising such trans-splicing molecule, or the cell comprising the trans-splicing molecule or recombinant vector is the only active ingredient in a pharmaceutical composition of the present invention. In other embodiments, the pharmaceutical composition further comprises one or more biologically active agents. Examples of suitable biologically active agents include, but are not limited to, anti-inflammatory agents, immunomodulatory agents, analgesics, antimicrobial agents, antibacterial agents, antibiotics, antioxidants, antiseptic agents, and combinations thereof. Where necessary or desired, a pharmaceutical composition may further include a local anaesthetic to ease pain at the site of injection.

In such pharmaceutical compositions, the nucleic acid OPA1 pre-mRNA trans-splicing molecule, the recombinant vector, or the cell and the at least one additional biologically active agent may be combined in one or more preparations for simultaneous, separate or sequential administration. More specifically, an inventive composition may be formulated in such a way that the nucleic acid OPA1 pre-mRNA trans-splicing molecule, the recombinant vector, or the cell and the additional biologically active agent(s) can be administered together or independently from each other. For example, the nucleic acid OPA1 pre-mRNA trans-splicing molecule, the recombinant vector, or the cell and the additional biologically active agent(s) can be formulated together in a single pharmaceutical composition. Alternatively, they may be maintained (e.g., in different compositions and/or containers) and administered separately, thereby constituting a pharmaceutical kit or pack.

### 2. Pharmaceutical Kits

In another aspect, the present invention provides a pharmaceutical kit comprising one or more containers (e.g., vials, ampoules, test tubes, flasks or bottles) containing one or more ingredients of an inventive pharmaceutical composition, allowing administration of a nucleic acid OPA1 pre-mRNA trans-splicing molecule described herein, or a recombinant vector comprising such trans-splicing molecule, or a cell comprising the trans-splicing molecule or the recombinant vector, to a subject in need thereof, for therapeutic purpose. Alternatively, the component for production or assembly of a pharmaceutical composition, including the nucleic acid OPA1 pre-mRNA trans-splicing molecule, carrier(s), rAAV particles, surfactants, and the like, as well as suitable laboratory hardware to prepare the composition may be incorporated into a kit.

Different ingredients of a pharmaceutical kit may be supplied in a solid (e.g., lyophilized) or liquid form. Each ingredient will generally be suitable as aliquoted in its respective container or provided in a concentrated form. Kits according to the invention may include media for the reconstitution of lyophilized ingredients. Individual containers of the kits will preferably be maintained in close confinement for commercial sale.

In certain embodiments, a pharmaceutical kit can include a device for administering the composition thereof, e.g., syringe needle, pen device, jet injector or another needle-free injector.

In certain embodiments, a pharmaceutical kit includes one or more additional biologically active agent(s). Optionally associated with the container(s) can be a notice or package insert in the form prescribed by a governmental agency regulating the manufacture, use or sale of pharmaceutical or biological products, which notice reflects approval by the agency of manufacture, use or sale for human administration. The notice of package insert may contain instructions for use of a pharmaceutical composition according to methods of treatment disclosed herein.

An identifier, e.g., a bar code, radio frequency, ID tags, etc., may be present in or on the kit. The identifier can be used, for example, to uniquely identify the kit for purposes of quality control, inventory control, tracking movement between workstations, etc.

The invention will be further illustrated by the following figures and examples. However, these examples and figures should not be interpreted in any way as limiting the scope of the present invention.

### EXAMPLES

The following examples describe some of the preferred modes of making and practicing the present invention. However, it should be understood that the examples are for illustrative purposes only and are not meant to limit the scope of the invention. Furthermore, unless the description in an Example is presented in the past tense, the text, like the rest of the specification, is not intended to suggest that experiments were actually carried out or data were actually obtained.

### Selection of the OPA1 Trans-Splicing Targeted Region

The Homo sapiens (Hs) OPA1 intron 8-9 (193,637,282 to 193,637,951)/EXON 9 (ENSE00003604184: 193,637,952 to 193,638,065) genomic sequence, defined as the human OPA1 trans-splicing targeted sequence, was chosen as the region to hybridize to via antisense binding domains. Homo sapiens OPA1 intron 8-9 (670 bp) and Homo sapiens OPA1 EXON 9 (114 bp) are respectively indicated in lowercases and uppercases in the following 784-nucleotides sequence. Underlined nucleotides represent the forward primer (5′-gtatgtgaaaaattgatagtgaacttgcc-3′=SEQ ID NO: 43) and reverse primer (5′-CTTAACTGGAGAACGTGTCATCATC-3′=SEQ ID NO: 44) used for PCR-amplification. The amplified 784-nucleotides fragment was used as a template for antisense binding domains genesis.

SEQ ID NO: 1
gtatgtgaaaaattgatagtgaacttgccaattagcaaaaaaaga
agcagcttagcttcctaaaaattatgtgtatatatgtacacatac
acatatatacatactagatgtaggcatttatattttttatgtaat
cttacatgttccaagtaatgtcttaagcaatattatttgactatt
ttagttcattataaatattaataaatataagtacattatatttat
gagttactgtatgtgttataaaggaagatatttggctttatatgt
cttaatattagtaatatttaaatactgaacagtggattaaattag
ccatatgcgtgaaatttaagctaatagaattgaaaatgtgtttgt
aaacagtaaactagctatggaaaagatttatggaaagttaataac
ctggttttagaaatactggtttaaattagcacaagtttttaaaat
aaaaattaggctataaacagtggatccagttatagttttgctgtt
cctattttcaatgtgcacacatgcatcaatcaccatttttttacg
gattttaaaatattttttcacctgtagaaattttaaaagactaaa
aaactcagagcagcattacaaataggttttaattttaatttggta
tcagaaaaatatgaataagtgttctttgttttgtgggaagGTTGT
TGTGGTTGGAGATCAGAGTGCTGGAAAGACTAGTGTGTTGGAAAT
GATTGCCCAAGCTCGAATATTCCCAAGAGGATCTGGGGAGATGAT
GACACGTTCTCCAGTTAAG

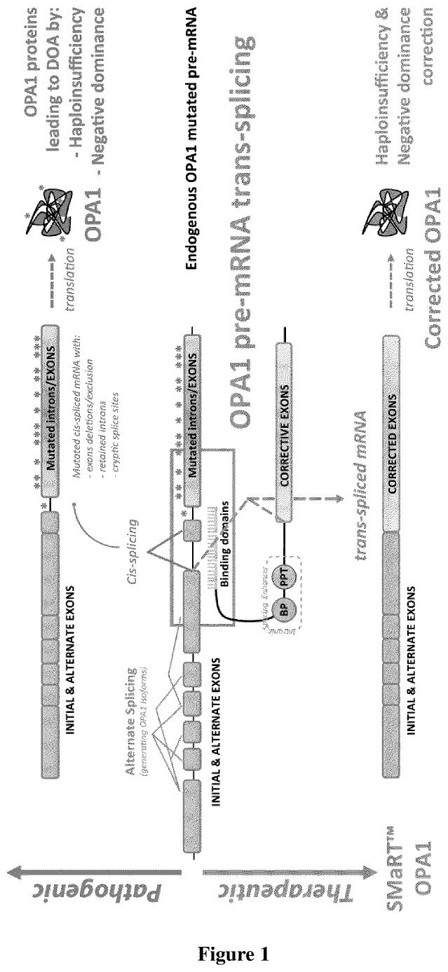

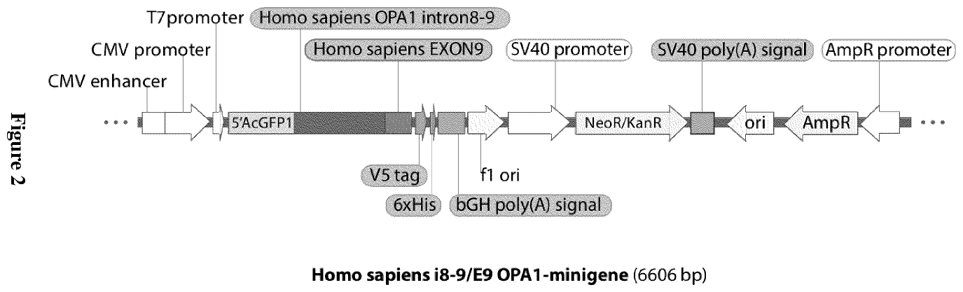

The HsOPA1 intron 8-9/EXON9 (193,637,282-193,638,065) sequence was chosen because of:
Its downstream position to OPA1 alternate spliced exons (4, 4b, 5b) and its upstream position to the pathogenic variants registered in OPA1-related Dominant Optic Atrophy (DOA) patients, OPA1-related Dominant Optic Atrophy Plus (DOA+) patients and patients with Behr's syndrome (see FIG. 1). This targeted region warranties both the conservation and the trans-splicing of the eight OPA1 pre-mRNA transcripts, to correct the eight different OPA1 mRNAs and corresponding OPA1 protein isoforms; and
Its size, which allows the generation of a wide diversity of antisense binding domains.

The HsOPA1 intron 8-9/EXON9 (193,637,282-193,638,065) sequence was also PCR-amplified with flanking primers:
forward primer:

(SEQ ID NO: 45)
5′-GCCGAGGTAAGAGATATCGTATGTGAAAAATTGAT
AGTGAACTTG-3′;
and
reverse primer:
(SEQ ID NO: 46)
5′-TAGACTCGAGCGGCCGCCTTAACTGGAGAACGTGT
CATC-3′

for HsOPA1 intron 8-9/EXON9 OPA1-minigene edition used in a minigene GFP assay (see FIG. 2). The resulting amplicon (819 bp) was cloned downstream to the 5′ half part of the AcGFP1 encoding sequence (336 bp), between EcoRV and NotI restriction sites, through the Gibson cloning assembly method, and confirmed by Sanger-sequencing using the following sequencing primers:

Seq HsOPA1-MG Fw1
(SEQ ID NO: 47)
5′-ATCTTCTTCGAGGATGACGGCAA-3′
Seq HsOPA1-MG Fw2:
(SEQ ID NO: 48)
5′-GCTATGGAAAAGATTTATGGAAAGTTAATAACCTGG-3′
Seq HsOPA1-MG Rv1:
SEQ ID NO: 49)
5′-GTTTAAACTCAATGGTGATGGTGATGATGA-3′
Seq HsOPA1-MG Rv2:
(SEQ ID NO: 50)
5′-AAACTATAACTGGATCCACTGTTTATAGCCTAA-3′

### Genesis of OPA1 Antisense Binding Domains

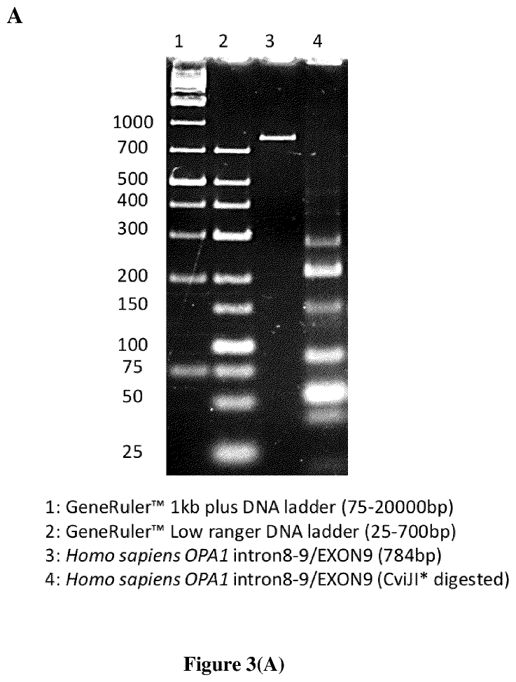

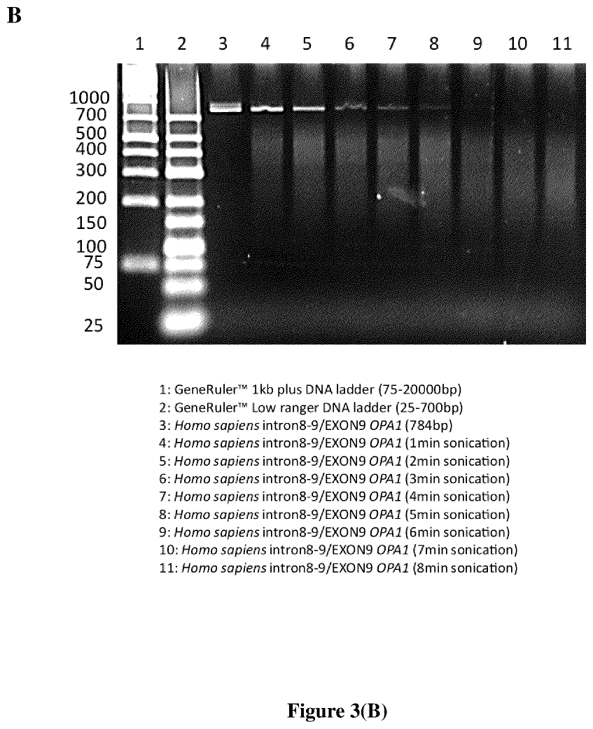

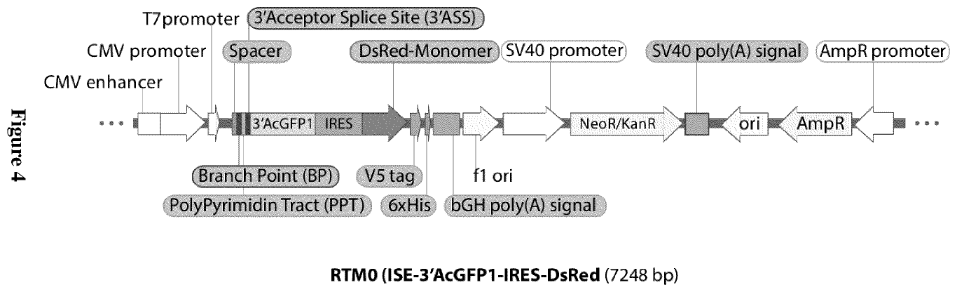

Starting from the HsOPA1 intron 8-9/EXON9 (193,637,282-193,638,065) PCR-amplified sequence (784 bp), the methods described by Bauer et al. (Methods Mol. Biol., 2013, 961:441-455) and Murauer et al. (Hum. Gene Ther. Methods, 2013, 24 (1): 19-27) were used to create banks of antisense binding domains through a CviJI* (5′-PuG [symbol] CPy-3′) endonuclease digestion or sonication (1 minute per 100 bp). CviJI* digested fragments ranked from 5 to 500 bp and sonicated fragments from 25 to 700 bp (see FIG. 3).

An additional bank of antisense binding domains was generated by rational PCR with intro 8-9/EXON9 (193,637,282-193,638,065) Homo sapiens OPA1 sequence as template and combination of the flanked PCR primers presented in Table 1 for downstream Gibson cloning assembly into RTM0 plasmid (see plasmid map in FIG. 4).

TABLE 1
Primers used for the rational PCR antisense binding domains genesis. Primers are flanked by Clal or EcoRI restriction sites and recipient plasmid sequence for efficient Gibson cloning assembly into RTMO plasmid.

| Forward Primers (5′-3′) | SEQ ID NOS: |
|---|---|
| TTCTCGTTAACATCGATGGTATCAGAAAA | SEQ ID NO: 51 |
| ATATGAATAAGTGTTCTTTGTTTTGTGG | |
| TTCTCGTTAACATCGATCCTGTAGAAATT | SEQ ID NO: 52 |
| TTAAAAGACTAAAAAACTCAGAGC | |
| TTCTCGTTAACATCGATGCATCAATCACC | SEQ ID NO: 53 |
| ATTTTTTTACGGATTTTAAAATATTTTTT | |
| CACC | |
| TTCTCGTTAACATCGATGCACACATGCAT | SEQ ID NO: 54 |
| CAATCACCATTTTTTTACG | |
| Reverse Primers (5′-3′) | SEQ ID NOS: |
| ACCCTTCACCGAATTCGCAATCATTTCC | SEQ ID NO: 55 |
| AACACACTAGTCTTTCC | |
| ACCCTTCACCGAATTCCCAGATCCTCTT | SEQ ID NO: 56 |
| GGGAATATTCGAGC | |
| ACCCTTCACCGAATTCCTTAACTGGAGA | SEQ ID NO: 57 |
| ACGTGTCATCATCTCC | |

### Cloning of OPA1 Antisense-Binding Domains into RTM0 Plasmid for the Minigene Assay

400 putative antisense binding domains to Homo sapiens OPA1 intron 8-9/EXON9 (193,637,282-193,638,065) generated by CviJI* digestion or sonication were subcloned, according to Murauer et al. (Hum. Gene Ther. Methods, 2013, 24 (1): 19-27), into the RTM0 plasmid (i.e., a plasmid without any antisense binding domains) at the HpaI blunt cloning site, while those generated by rational PCR were subcloned between EcoRI and ClaI cohesive cloning sites for OPA1-specific RTMs edition (see FIG. 4). The RTM0 plasmid, which was published in Bauer et al. (Methods In Molecular Biology, 2013, vol. 961, DOI 10.1007/978-1-62703-227-8_30) and Murauer et al., (Hum. Gene Ther. Methods, 2013, 24 (1): 19-27), was a gift from the group of Ulrich Koller (Austria).

These Homo sapiens OPA1 antisense binding domains were linked to an Intronic Splicing Enhancer (ISE) cassette included in the RTM0 plasmid and containing a 27-bp spacer, a 8-bp branch point (BP), a 16-bp PolyPyrimidine Tract (PPT), a 3′ Acceptor Splice Site (3′ASS) as shown on FIG. 5.

Cloning of the OPA1 antisense binding domains was confirmed by Sanger-sequencing using forward (5′-GGCTAACTAGAGAACCCACTGC-3′-SEQ ID NO: 58) and reverse (5′-CCATCCTCCTTGAAATCGGTGC-3′-SEQ ID NO: 59) primers.

### Mechanistic Proof of Principle of Homo sapiens OPA1 Trans-Splicing and Selection of OPA1-Targeting RTMs Containing Antisense Binding Domains Inducing the Highest Trans-Splicing Rates

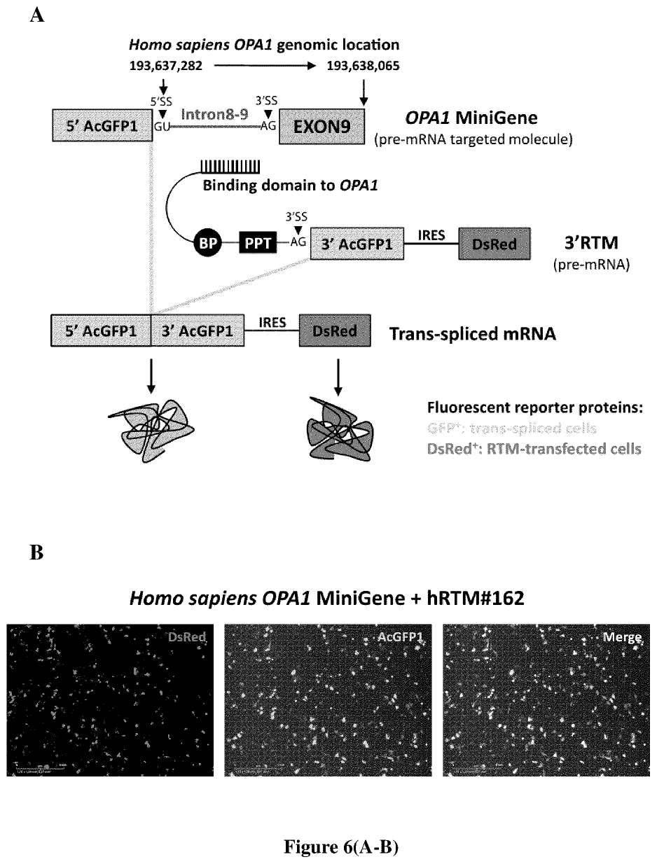

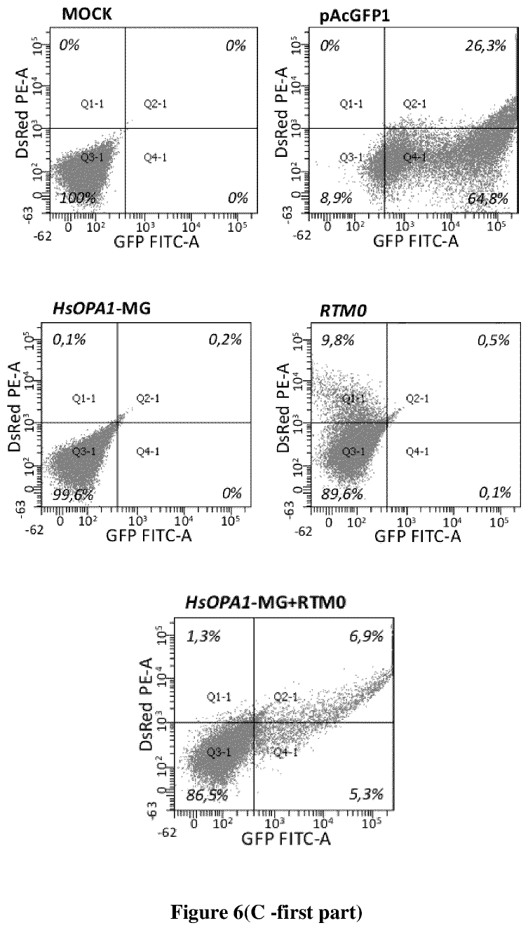

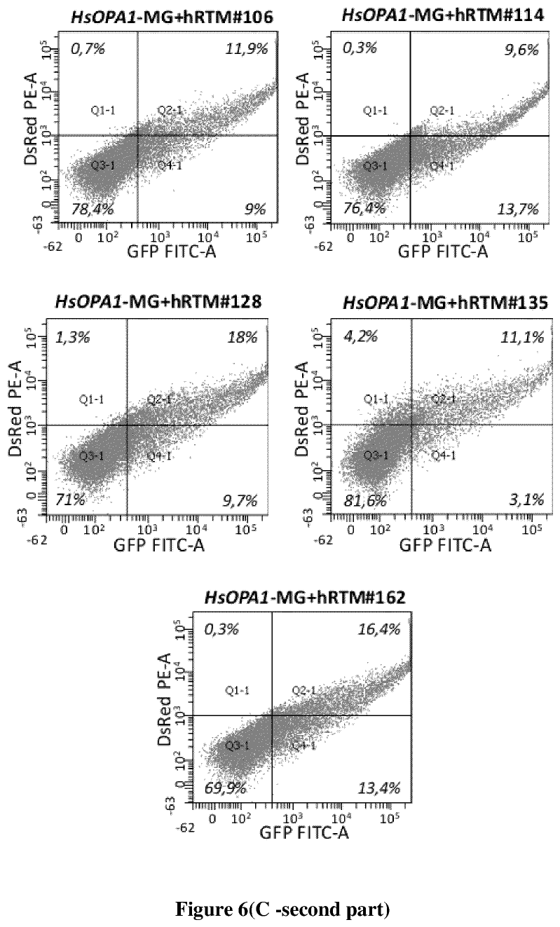

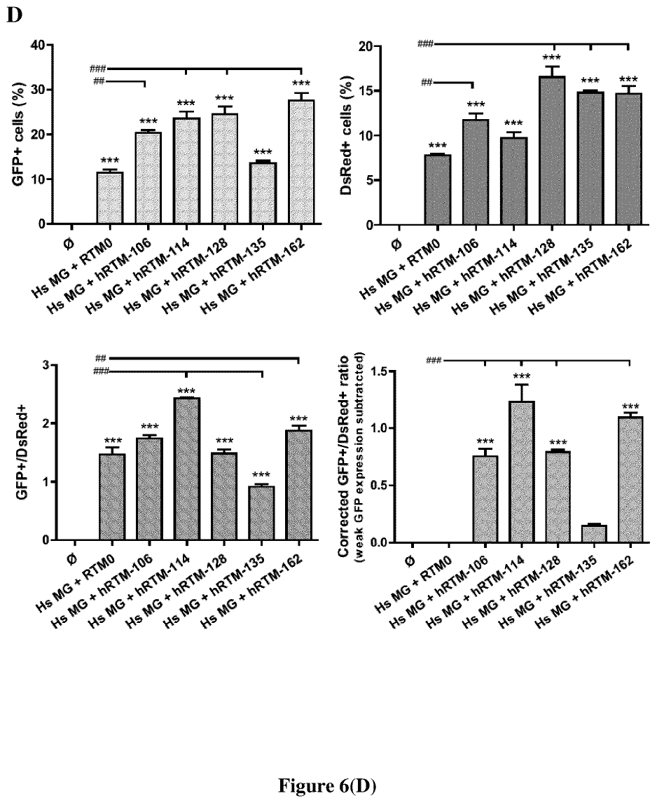

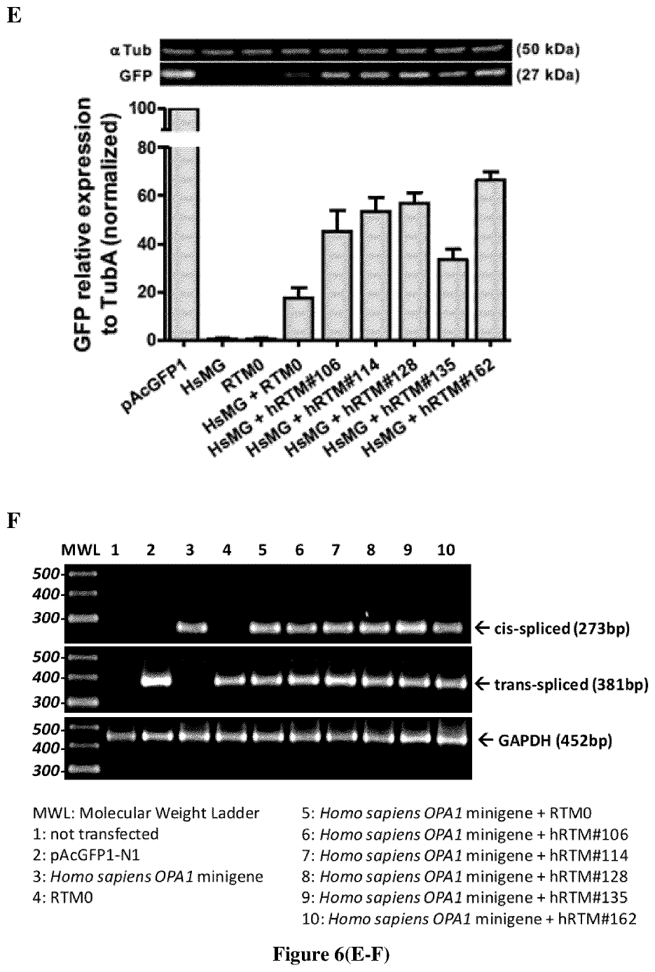

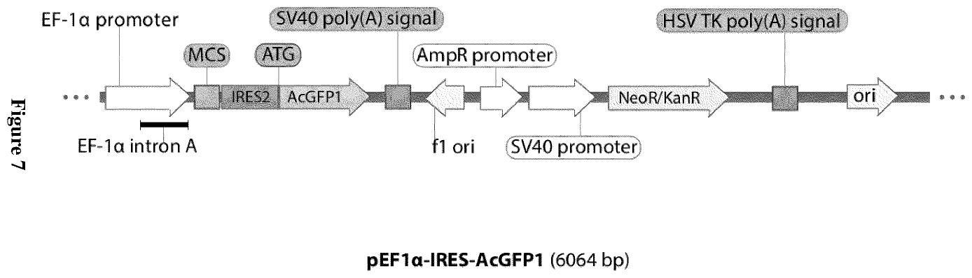

According to Murauer et al. (Hum. Gene Ther. Methods, 2013, 24 (1): 19-27) and to Koller et al. (Mol. Ther. Nucleic Acids, 2014, 3 (4): e157) for recommendations in the design of a highly functional RNA Trans-splicing Molecule, individual OPA1-specific RTMs were tested in the minigene GFP reconstitution assay for their ability to trans-splice the co-transfected targeted Homo sapiens intron 8-9/EXON 9 OPA1 minigene. Efficient antisense binding domains to Homo sapiens OPA1 intron 8-9/EXON9 (193,637,282-193,638,065) were selected as those inducing the highest OPA1 trans-splicing rates revealed by the highest proportion of GFP expressing cells (see FIG. 6).

### Identification of Efficient Binding Domains to Homo sapiens OPA1 Trans-Splicing

The mechanistic proof of principle using the OPA1-minigene assay revealed efficient binding domains for OPA1 trans-splicing:
BD #106 (250 nt), which is a multiple binding domain with the following sequence. In the sequence below, the sign “/” separates the different binding domains.

(SEQ ID NO: 6)
cctaatttttattttaaaaacttgtgctaatttaaaccagtattt
ctaaaaccaggttattaactttccataaatcttttccatag/ctg
cttctttttttgctaattggcaagttcactatcaatttttcacat
ac/ctttatatgtcttaatattagtaatatttaaatactgaacag
tggattaaattag/cttaaatttcacgcatatgg/ctagtttact
gtttacaaacacattttcaattctattag.

It hybridizes to the human genome at: 3:193,637,656-3:193,637,741 & 3:193,637,282-3:193,637,331 & 3:193,637,597-3:193,637,616 & 3:193,637,617-3:193,637,655 The corresponding 5′-3′ sequences are:

(SEQ ID NO: 60)
ctatggaaaagatttatggaaagttaataacctggttttagaaat
actggtttaaattagcacaagtttttaaaataaaaattagg/gta
tgtgaaaaattgatagtgaacttgccaattagcaaaaaaagaagc
ag/ccatatgcgtgaaatttaag/ctaatagaattgaaaatgtgt
ttgtaaacagtaaactag.

BD #114 (55 nt), which is a single binding domain and has the following sequence:

(SEQ ID NO: 2)
ctaatttaatccactgttcagtatttaaatattactaatattaag
acatataaag.

It hybridizes to the human genome at: 3:193,637,542-3:193,637,596 The corresponding 5′-3′ sequence is:

(SEQ ID NO: 61)
ctttatatgtcttaatattagtaatatttaaatactgaacagtgg
attaaattag.

BD #128 (86 nt), which is a single binding domain and has the following sequence:

(SEQ ID NO: 3)
cctaatttttattttaaaaacttgtgctaatttaaaccagtattt
ctaaaaccaggttattaactttccataaatcttttccatag.

It hybridizes to the human genome at: 3:193,637,656-3:193,637,741 The corresponding 5′-3′ sequence is:

(SEQ ID NO: 62)
ctatggaaaagatttatggaaagttaataacctggttttagaaat
actggtttaaattagcacaagtttttaaaataaaaattagg.

BD #135 (105 nt), which is a double binding domain (1× antisense+1× sense). In the sequence, the sign “/” separates the two different binding domains.

(SEQ ID NO: 5)
ctgcttctttttttgctaattggcaagttcactatcaatttttca
catac/ctttatatgtcttaatattagtaatatttaaatactgaa
cagtggattaaattag.

It hybridizes to the human genome at: 3:193,637,282-3:193,637,331 only. The corresponding 5′-3′ sequence is:

(SEQ ID NO: 63)
gtatgtgaaaaattgatagtgaacttgccaattagcaaaaaaaga
agcag.

BD #162 (143 nt), which is a single binding domain and has the following sequence:

(SEQ ID NO: 4)
taatgctgctctgagttttttagtcttttaaaatttctacaggtg
aaaaaatattttaaaatccgtaaaaaaatggtgattgatgcatgt
gtgcacattgaaaataggaacagcaaaactataactggatccact
gtttatag.

It hybridizes to the human genome at: 3:193,637,742-3:193,637,884. The corresponding 5′-3′ sequence is:

(SEQ ID NO: 64)
ctataaacagtggatccagttatagttttgctgttcctattttca
atgtgcacacatgcatcaatcaccatttttttacggattttaaaa
tattttttcacctgtagaaattttaaaagactaaaaaactcagag
cagcatta.

Other antisense binding domains have also been generated by rational PCR. See primers used presented in Table 1. These antisense binding domains for OPA1 trans-splicing are:
BD #100 (100 nt), which is a single binding domain and has the following sequence:

(SEQ ID NO: 7)
gcaatcatttccaacacactagtctttccagcactctgatctcca
accacaacaaccttcccacaaaacaaagaacacttattcatattt
ttctgatacc.

It hybridizes to the human genome at: 3:193,637,908-3:193,638,007. The corresponding 5′-3′ sequence is:

(SEQ ID NO: 77)
ggtatcagaaaaatatgaataagtgttctttgttttgtgggaagG
TTGTTGTGGTTGGAGATCAGAGTGCTGGAAAGACTAGTGTGTTGG
AAATGATTGC.

BD #130 (130 nt), which is a single binding domain and has the following sequence:

(SEQ ID NO: 8)
ccagatcctcttgggaatattcgagcttgggcaatcatttccaac
acactagtctttccagcactctgatctccaaccacaacaaccttc
ccacaaaacaaagaacacttattcatatttttctgatacc.

It hybridizes to the human genome at: 3:193,637,908-3:193,638,037. The corresponding 5′-3′ sequence is:

(SEQ ID NO: 78)
ggtatcagaaaaatatgaataagtgttctttgttttgtgggaagG
TTGTTGTGGTTGGAGATCAGAGTGCTGGAAAGACTAGTGTGTTGG
AAATGATTGCCCAAGCTCGAATATTCCCAAGAGGATCTGG.

BD #158 (158 nt), which is a single binding domain and has the following sequence:

(SEQ ID NO: 9)
cttaactggagaacgtgtcatcatctccccagatcctcttgggaa
tattcgagcttgggcaatcatttccaacacactagtctttccagc
actctgatctccaaccacaacaaccttcccacaaaacaaagaaca
cttattcatatttttctgatacc.

It hybridizes to the human genome at: 3:193,637,908-3:193,638,065. The corresponding 5′-3′ sequence is:

(SEQ ID NO: 79)
ggtatcagaaaaatatgaataagtgttctttgttttgtgggaagG
TTGTTGTGGTTGGAGATCAGAGTGCTGGAAAGACTAGTGTGTTGG
AAATGATTGCCCAAGCTCGAATATTCCCAAGAGGATCTGGGGAGA
TGATGACACGTTCTCCAGTTAAG.

BD #166 (166 nt), which is a single binding domain and has the following sequence:

(SEQ ID NO: 10)
GCAATCATTTCCAACACACTAGTCTTTCCAGCACTCTGATCTCCA
ACCACAACAACcttcccacaaaacaaagaacacttattcatattt
ttctgataccaaattaaaattaaaacctatttgtaatgctgctct
gagttttttagtcttttaaaatttctacagg.

It hybridizes to the human genome at: 3:193,637,842-3:193,638,007. The corresponding 5′-3′ sequence is:

(SEQ ID NO: 80)
cctgtagaaattttaaaagactaaaaaactcagagcagcattaca
aataggttttaattttaatttggtatcagaaaaatatgaataagt
gttctttgttttgtgggaagGTTGTTGTGGTTGGAGATCAGAGTG
CTGGAAAGACTAGTGTGTTGGAAATGATTGC.

BD #196 (196 nt), which is a single binding domain and has the following sequence:

(SEQ ID NO: 11)
ccagatcctcttgggaatattcgagcttgggcaatcatttccaac
acactagtctttccagcactctgatctccaaccacaacaaccttc
ccacaaaacaaagaacacttattcatatttttctgataccaaatt
aaaattaaaacctatttgtaatgctgctctgagttttttagtctt
ttaaaatttctacagg.

It hybridizes to the human genome at: 3:193,637,842-193,638,037. The corresponding 5′-3′ sequence is:

(SEQ ID NO: 81)
cctgtagaaattttaaaagactaaaaaactcagagcagcattaca
aataggttttaattttaatttggtatcagaaaaatatgaataagt
gttctttgttttgtgggaagGTTGTTGTGGTTGGAGATCAGAGTG
CTGGAAAGACTAGTGTGTTGGAAATGATTGCCCAAGCTCGAATAT
TCCCAAGAGGATCTGG.

BD #224 (224 nt), which is a single binding domain and has the following sequence:

(SEQ ID NO: 12)
cttaactggagaacgtgtcatcatctccccagatcctcttgggaa
tattcgagcttgggcaatgatttccaacacactagtctttccagc
actctgatctccaaccacaacaaccttcccacaaaacaaagaaca
cttattcatatttttctgataccaaattaaaattaaaacctattt
gtaatgctgctctgagttttttagtcttttaaaatttctacagg.

It hybridizes to the human genome at: 3:193,637,842-3:193,638,065. The corresponding 5′-3′ sequence is:

(SEQ ID NO: 82)
cctgtagaaattttaaaagactaaaaaactcagagcagcattaca
aataggttttaattttaatttggtatcagaaaaatatgaataagt
gttctttgttttgtgggaagGTTGTTGTGGTTGGAGATCAGAGTG
CTGGAAAGACTAGTGTGTTGGAAATGATTGCCCAAGCTCGAATAT
TCCCAAGAGGATCTGGGGAGATGATGACACGTTCTCCAGTTAAG.

BD #209 (209 nt), which is a single binding domain and has the following sequence:

(SEQ ID NO: 13)
gcaatcatttccaacacactagtctttccagcactctgatctcca
accacaacaaccttcccacaaaacaaagaacacttattcatattt
ttctgataccaaattaaaattaaaacctatttgtaatgctgctct
gagttttttagtcttttaaaatttctacaggtgaaaaaatatttt
aaaatccgtaaaaaaatggtgattgatgc.

It hybridizes to the human genome at: 3:193,637,799-3:193,638,007. The corresponding 5′-3′ sequence is:

(SEQ ID NO: 83)
gcatcaatcaccatttttttacggattttaaaatattttttcacctgta
gaaattttaaaagactaaaaaactcagagcagcattacaaataggtttt
aattttaatttggtatcagaaaaatatgaataagtgttctttgttttgt
gggaagGTTGTTGTGGTTGGAGATCAGAGTGCTGGAAAGACTAGTGTGT
TGGAAATGATTGC.

BD #239 (239 nt), which is a single binding domain and has the following sequence:

(SEQ ID NO: 14)
Ccagatcctcttgggaatattcgagcttgggcaatcatttccaacacac
tagtctttccagcactctgatctccaaccacaacaaccttcccacaaaa
caaagaacacttattcatatttttctgataccaaattaaaattaaaacc
tatttgtaatgctgctctgagttttttagtcttttaaaatttctacagg
tgaaaaaatattttaaaatccgtaaaaaaatggtgattgatgc.

It hybridizes to the human genome at: 3:193,637,799-3:193,638,037. The corresponding 5′-3′ sequence is:

(SEQ ID NO: 84)
gcatcaatcaccatttttttacggattttaaaatattttttcacctgta
gaaattttaaaagactaaaaaactcagagcagcattacaaataggtttt
aattttaatttggtatcagaaaaatatgaataagtgttctttgttttgt
gggaagGTTGTTGTGGTTGGAGATCAGAGTGCTGGAAAGACTAGTGTGT
TGGAAATGATTGCCCAAGCTCGAATATTCCCAAGAGGATCTGG.

BD #267 (267 nt), which is a single binding domain and has the following sequence:

(SEQ ID NO: 15)
cttaactggagaacgtgtcatcatctccccagatcctcttgggaatatt
cgagcttgggcaatcatttccaacacactagtctttccagcactctgat
ctccaaccacaacaaccttcccacaaaacaaagaacacttattcatatt
tttctgataccaaattaaaattaaaacctatttgtaatgctgctctgag
ttttttagtcttttaaaatttctacaggtgaaaaaatattttaaaatcc
gtaaaaaaatggtgattgatgc.

It hybridizes to the human genome at: 3:193,637,799-3:193,638,065. The corresponding 5′-3′ sequence is:

(SEQ ID NO: 85)
gcatcaatcaccatttttttacggattttaaaatattttttcacctgta
gaaattttaaaagactaaaaaactcagagcagcattacaaataggtttt
aattttaatttggtatcagaaaaatatgaataagtgttctttgttttgt
gggaagGTTGTTGTGGTTGGAGATCAGAGTGCTGGAAAGACTAGTGTGT
TGGAAATGATTGCCCAAGCTCGAATATTCCCAAGAGGATCTGGGGAGAT
GATGACACGTTCTCCAGTTAAG.

BD #217 (217 nt), which is a single binding domain and has the following sequence:

(SEQ ID NO: 16
gcaatcatttccaacacactagtctttccagcactctgatctccaacca
caacaaccttcccacaaaacaaagaacacttattcatatttttctgata
ccaaattaaaattaaaacctatttgtaatgctgctctgagttttttagt
cttttaaaatttctacaggtgaaaaaatattttaaaatccgtaaaaaaa
tggtgattgatgcatgtgtgc.

It hybridizes to the human genome at: 3:193,637,791-3:193,638,007. The corresponding 5′-3′ sequence is:

(SEQ ID NO: 86)
gcacacatgcatcaatcaccatttttttacggattttaaaatatttttt
cacctgtagaaattttaaaagactaaaaaactcagagcagcattacaaa
taggttttaattttaatttggtatcagaaaaatatgaataagtgttctt
tgttttgtgggaagGTTGTTGTGGTTGGAGATCAGAGTGCTGGAAAGAC
TAGTGTGTTGGAAATGATTGC.

BD #247 (247 nt), which is a single binding domain and has the following sequence:

(SEQ ID NO: 17)
ccagatcctcttgggaatattcgagcttgggcaatcatttccaacacac
tagtctttccagcactctgatctccaaccacaacaaccttcccacaaaa
caaagaacacttattcatatttttctgataccaaattaaaattaaaacc
tatttgtaatgctgctctgagttttttagtcttttaaaatttctacagg
tgaaaaaatattttaaaatccgtaaaaaaatggtgattgatgcatgtgt
gc.

It hybridizes to the human genome at: 3:193,637,791-3:193,638,037. The corresponding 5′-3′ sequence is:

(SEQ ID NO: 87)
gcacacatgcatcaatcaccatttttttacggattttaaaatatttttt
cacctgtagaaattttaaaagactaaaaaactcagagcagcattacaaa
taggttttaattttaatttggtatcagaaaaatatgaataagtgttctt
tgttttgtgggaagGTTGTTGTGGTTGGAGATCAGAGTGCTGGAAAGAC
TAGTGTGTTGGAAATGATTGCCCAAGCTCGAATATTCCCAAGAGGATCT
GG.

BD #275 (275 nt), which is a single binding domain and has the following sequence:

(SEQ ID NO: 18)
ttacctggagaacgtgtcatcatctccccagatcctcttgggaatattc
gagcttgggcaatcatttccaacacactagtctttccagcactctgatc
tccaaccacaacaaccttcccacaaaacaaagaacacttattcatattt
ttctgataccaaattaaaattaaaacctatttgtaatgctgctctgagt
tttttagtcttttaaaatttctacaggtgaaaaaatattttaaaatccg
taaaaaaatggtgattgatgcatgtgtgc.

It hybridizes to the human genome at: 3:193,637,791-3:193,638,060. The corresponding 5′-3′ sequence is:

(SEQ ID NO: 88)
gcacacatgcatcaatcaccatttttttacggattttaaaatatttttt
cacctgtagaaattttaaaagactaaaaaactcagagcagcattacaaa
taggttttaattttaatttggtatcagaaaaatatgaataagtgttctt
tgttttgtgggaagGTTGTTGTGGTTGGAGATCAGAGTGCTGGAAAGAC
TAGTGTGTTGGAAATGATTGCCCAAGCTCGAATATTCCCAAGAGGATCT
GGGGAGATGATGACACGTTCTCCAGTTAAG.

Cloning into a Therapeutic Vector

The preselected OPA1-RTMs were PCR-amplified for Gibson assembly with the optimized wild-type OPA1 cDNA sequence using the primers presented in Table 2 below.

TABLE 2
Primers used for generating therapeutic OPA1 RNA trans-splicing molecules
according to the present invention.

| Fragment | Forward primer | Reverse primer |
|---|---|---|
| BD#106 + ISE | cgagctcaagcttcgaattccctaa | ctgatcacctacgactactacctg |
| BD#128 + ISE | tttttattttaaaaacttgtgc | cagaaaaaaaagaagagg |
| | (SEQ ID NO: 65) | (SEQ ID NO: 70) |
| BD#114 + ISE | cgagctcaagcttcgaattcctaat | |
| | ttaatccactgttcagtattt | |
| | (SEQ ID NO: 66) | |
| BD#135 + ISE | cgagctcaagcttcgaattcctgct | |
| | tctttttttgctaattg (SEQ ID | |
| | NO: 67) | |
| BD#162 + ISE | cgagctcaagcttcgaattctaatg | |
| | ctgctctgagttttttag (SEQ | |
| | ID NO: 68) | |
| Homo | ctgcagGTAGTAGTCGTAGGTGATC | cgggcccgcggtaccgtcgacttaI |
| sapiens | AGAGTGCTGGAAAG (SEQ ID | tttctcctgatgaagagcttc |
| OPA1 DNA | NO: 69) | (SEQ ID NO: 71) |

It generated OPA1 antisense binding domains fused to ISE with the sequences presented below, wherein the binding domain (BD) is in bold, the spacer is underlined, the branch point (BP) is in bold and underlined, the Poly-Pyrimidin Track (PPT) is in italics, and the 3′ Acceptor Splicing Site (3′ASS) is in italics and underlined.

-BD#106 + ISE (SEQ ID NO: 72):
cgagctcaagcttcgaattccctaatttttattttaaaaacttgtgcta
atttaaaccagtatttctaaaaccaggttattaactttccataaatctt
ttccatag/ctgcttctttttttgctaattggcaagttcactatcaatt
tttcacatac/ctttatatgtcttaatattagtaatatttaaatactga
acagtggattaaattag/cttaaatttcacgcatatgg/ctagtttact
gtttacaaacacattttcaattctattagaacgagaacattattatagc
gttgctcgagtactaactggtacctcttcttttttttctgcagGTAGTA
GTCGTAGGTGATCAG
-BD#114 + ISE (SEQ ID NO: 73):
cgagctcaagcttcgaattcctaatttaatccactgttcagtatttaaa
tattactaatattaagacatataaagaacgagaacattattatagcgt
tgctcgagtactaactggtacctcttcttttttttctgcagGTAGTAGT
CGTAGGTGATCAG
-BD#128 + ISE (SEQ ID NO: 74):
cgagctcaagcttcgaattccctaatttttattttaaaaacttgtgcta
atttaaaccagtatttctaaaaccaggttattaactttccataaatctt
ttccatagaacgagaacattattatagcgttgctcgagtactaactggt
acctcttcttttttttctgcagGTAGTAGTCGTAGGTGATCAG
-BD#135 + ISE (SEQ ID NO: 75):
cgagctcaagcttcgaattcctgcttctttttttgctaattggcaagtt
cactatcaatttttcacatacctttatatgtcttaatattagtaatatt
taaatactgaacagtggattaaattagaacgagaacattattatagcgt
tgctcgagtactaactggtacctcttcttttttttctgcagGTAGTAGT
CGTAGGTGATCAG
-BD#162 + ISE (SEQ ID NO: 76):
cgagctcaagcttcgaattctaatgctgctctgagttttttagtctttt
aaaatttctacaggtgaaaaaatattttaaaatccgtaaaaaaatggtg
attgatgcatgtgtgcacattgaaaataggaacagcaaaactataactg
gatccactgtttatagaacgagaacattattatagcgttgctcgagtac
taactggtacctcttcttttttttctgcagGTAGTAGTCGTAGGTGATC
AG

It also generated a 2013 nucleotides corrective OPA1 cDNA (the modified wild-type Homo sapiens OPA1 cDNA from EXON9 to the STOP codon), which is 5′-compatible with the 3′-end of the upstream binding domains+ISE fragment for Gibson cloning assembly.

(SEQ ID NO: 25)
ctgcagGTAGTAGTCGTAGGTGATCAGAGTGCTGGAAAGACTAGTGTGTTGGAAATGATTGCCCAAG
CTCGAATATTCCCAAGAGGATCTGGGGAGATGATGACACGTTCTCCAGTTAAGGTGACTCTGAGTGA
AGGTCCTCACCATGTGGCCCTATTTAAAGATAGTTCTCGGGAGTTTGATCTTACCAAAGAAGAAGAT
CTTGCAGCATTAAGACATGAAATAGAACTTCGAATGAGGAAAAATGTGAAAGAAGGCTGTACCGTTA
GCCCTGAGACCATATCCTTAAATGTAAAAGGCCCTGGACTACAGAGGATGGTGCTTGTTGACTTACC
AGGTGTGATTAATACTGTGACATCAGGCATGGCTCCTGACACAAAGGAAACTATTTTCAGTATCAGC
AAAGCTTACATGCAGAATCCTAATGCCATCATACTGTGTATTCAAGATGGATCTGTGGATGCTGAAC
GCAGTATTGTTACAGACTTGGTCAGTCAAATGGACCCTCATGGAAGGAGAACCATATTCGTTTTGAC
CAAAGTAGACCTGGCAGAGAAAAATGTAGCCAGTCCAAGCAGGATTCAGCAGATAATTGAAGGAAAG
CTCTTCCCAATGAAAGCTTTAGGTTATTTTGCTGTTGTAACAGGAAAAGGGAACAGCTCTGAAAGCA
TTGAAGCTATAAGAGAATATGAAGAAGAGTTTTTTCAGAATTCAAAGCTCCTAAAGACAAGCATGCT
AAAGGCACACCAAGTGACTACAAGAAATTTAAGCCTTGCAGTATCAGACTGCTTTTGGAAAATGGTA
CGAGAGTCTGTTGAACAACAGGCTGATAGTTTCAAAGCAACACGTTTTAACCTTGAAACTGAATGGA
AGAATAACTATCCTCGCCTGCGGGAACTTGACCGGAATGAACTATTTGAAAAAGCTAAAAATGAAAT
CCTTGATGAAGTTATCAGTCTGAGCCAGGTTACACCAAAACATTGGGAGGAAATCCTTCAACAATCT
TTGTGGGAAAGAGTATCAACTCATGTGATTGAAAACATCTACCTTCCAGCTGCGCAGACCATGAATT
CAGGAACTTTTAACACCACAGTGGATATCAAGCTTAAACAGTGGACTGATAAACAACTTCCTAATAA
AGCAGTAGAGGTTGCTTGGGAGACCCTACAAGAAGAATTTTCCCGCTTTATGACAGAACCGAAAGGG
AAAGAGCATGATGACATATTTGATAAACTTAAAGAGGCTGTTAAGGAAGAAAGTATTAAACGACACA
AGTGGAATGACTTTGCGGAGGACAGCTTGAGGGTTATTCAACACAATGCTTTGGAAGACCGATCCAT
ATCTGATAAACAGCAATGGGATGCAGCTATTTATTTTATGGAAGAGGCTCTGCAGGCTCGTCTCAAG
GATACTGAAAATGCAATTGAAAACATGGTGGGTCCAGACTGGAAAAAGAGGTGGTTATACTGGAAGA
ATCGGACCCAAGAACAGTGTGTTCACAATGAAACCAAGAATGAATTGGAGAAGATGTTGAAATGTAA
TGAGGAGCACCCAGCTTATCTTGCAAGTGATGAAATAACCACAGTCCGGAAGAACCTTGAATCCCGA
GGAGTAGAAGTAGATCCAAGCTTGATTAAGGATACTTGGCATCAAGTTTATAGAAGACATTTTTTAA
AAACAGCTCTAAACCATTGTAACCTTTGTCGAAGAGGTTTTTATTACTACCAAAGGCATTTTGTAGA
TTCTGAGTTGGAATGCAATGATGTGGTCTTGTTTTGGCGTATACAGCGCATGCTTGCTATCACCGCA
AATACTTTAAGGCAACAACTTACAAATACTGAAGTTAGGCGATTAGAGAAAAATGTTAAAGAGGTAT
TGGAAGATTTTGCTGAAGATGGTGAGAAGAAGATTAAATTGCTTACTGGTAAACGCGTTCAACTGGC
GGAAGACCTCAAGAAAGTTAGAGAAATTCAAGAAAAACTTGATGCTTTCATTGAAGCTCTTCATCAG
GAGAAATAAgtcgacggtaccgcgggcccg.

The most potent Homo sapiens OPA1 trans-splicing binding domains were subcloned upstream to the modified wild-type OPA1 cDNA (2013 bp) into the bicistronic mammalian expression vector pEF1a-IRES-AcGFP1 (631971, TakaraBio, see FIG. 7), between EcoRI and SalI restriction sites, through the Gibson cloning assembly method. These constructs were transiently transfected in OPA1-related Dominant Optic Atrophy (ADOA) patients' fibroblasts for OPA1 haploinsufficiency rescue and mitochondrial fusion induction assays.

It generated therapeutic OPA1 pre-mRNA Trans-splicing Molecules whose sequences are: 5′-Binding Domain (BD)-Spacer-Branch Point (BP)-KpnI-PolyPyrimidin Tract (PPT)-3′ Acceptor Splicing Site (3′ASS)-OPA1 cDNA-3′:

-OPA1 RTMₜₕ#106:
(SEQ ID NO: 26)
cctaatttttattttaaaaacttgtgctaatttaaaccagtatttctaaaaccaggttattaac
tttccataaatcttttccatag/ctgcttctttttttgctaattggcaagttcactatcaattt
ttcacatac/ctttatatgtcttaatattagtaatatttaaatactgaacagtggattaaatta
g/cttaaatttcacgcatatgg/ctagtttactgtttacaaacacattttcaattctattagaa
cgagaacattattatagcgttgctcgagtactaactggtacctcttcttttttttctgcagGTAG
TAGTCGTAGGTGATCAGAGTGCTGGAAAGACTAGTGTGTTGGAAATGATTGCCCAAGCTCGAATATT
CCCAAGAGGATCTGGGGAGATGATGACACGTTCTCCAGTTAAGGTGACTCTGAGTGAAGGTCCTCAC
CATGTGGCCCTATTTAAAGATAGTTCTCGGGAGTTTGATCTTACCAAAGAAGAAGATCTTGCAGCAT
TAAGACATGAAATAGAACTTCGAATGAGGAAAAATGTGAAAGAAGGCTGTACCGTTAGCCCTGAGAC
CATATCCTTAAATGTAAAAGGCCCTGGACTACAGAGGATGGTGCTTGTTGACTTACCAGGTGTGATT
AATACTGTGACATCAGGCATGGCTCCTGACACAAAGGAAACTATTTTCAGTATCAGCAAAGCTTACA
TGCAGAATCCTAATGCCATCATACTGTGTATTCAAGATGGATCTGTGGATGCTGAACGCAGTATTGT
TACAGACTTGGTCAGTCAAATGGACCCTCATGGAAGGAGAACCATATTCGTTTTGACCAAAGTAGAC
CTGGCAGAGAAAAATGTAGCCAGTCCAAGCAGGATTCAGCAGATAATTGAAGGAAAGCTCTTCCCAA
TGAAAGCTTTAGGTTATTTTGCTGTTGTAACAGGAAAAGGGAACAGCTCTGAAAGCATTGAAGCTAT
AAGAGAATATGAAGAAGAGTTTTTTCAGAATTCAAAGCTCCTAAAGACAAGCATGCTAAAGGCACAC
CAAGTGACTACAAGAAATTTAAGCCTTGCAGTATCAGACTGCTTTTGGAAAATGGTACGAGAGTCTG
TTGAACAACAGGCTGATAGTTTCAAAGCAACACGTTTTAACCTTGAAACTGAATGGAAGAATAACTA
TCCTCGCCTGCGGGAACTTGACCGGAATGAACTATTTGAAAAAGCTAAAAATGAAATCCTTGATGAA
GTTATCAGTCTGAGCCAGGTTACACCAAAACATTGGGAGGAAATCCTTCAACAATCTTTGTGGGAAA
GAGTATCAACTCATGTGATTGAAAACATCTACCTTCCAGCTGCGCAGACCATGAATTCAGGAACTTT
TAACACCACAGTGGATATCAAGCTTAAACAGTGGACTGATAAACAACTTCCTAATAAAGCAGTAGAG
GTTGCTTGGGAGACCCTACAAGAAGAATTTTCCCGCTTTATGACAGAACCGAAAGGGAAAGAGCATG
ATGACATATTTGATAAACTTAAAGAGGCTGTTAAGGAAGAAAGTATTAAACGACACAAGTGGAATGA
CTTTGCGGAGGACAGCTTGAGGGTTATTCAACACAATGCTTTGGAAGACCGATCCATATCTGATAAA
CAGCAATGGGATGCAGCTATTTATTTTATGGAAGAGGCTCTGCAGGCTCGTCTCAAGGATACTGAAA
ATGCAATTGAAAACATGGTGGGTCCAGACTGGAAAAAGAGGTGGTTATACTGGAAGAATCGGACCCA
AGAACAGTGTGTTCACAATGAAACCAAGAATGAATTGGAGAAGATGTTGAAATGTAATGAGGAGCAC
CCAGCTTATCTTGCAAGTGATGAAATAACCACAGTCCGGAAGAACCTTGAATCCCGAGGAGTAGAAG
TAGATCCAAGCTTGATTAAGGATACTTGGCATCAAGTTTATAGAAGACATTTTTTAAAAACAGCTCT
AAACCATTGTAACCTTTGTCGAAGAGGTTTTTATTACTACCAAAGGCATTTTGTAGATTCTGAGTTG
GAATGCAATGATGTGGTCTTGTTTTGGCGTATACAGCGCATGCTTGCTATCACCGCAAATACTTTAA
GGCAACAACTTACAAATACTGAAGTTAGGCGATTAGAGAAAAATGTTAAAGAGGTATTGGAAGATTT
TGCTGAAGATGGTGAGAAGAAGATTAAATTGCTTACTGGTAAACGCGTTCAACTGGCGGAAGACCTC
AAGAAAGTTAGAGAAATTCAAGAAAAACTTGATGCTTTCATTGAAGCTCTTCATCAGGAGAAATAA;
-OPA1 RTMₜₕ#114:
(SEQ ID NO: 27)
ctaatttaatccactgttcagtatttaaatattactaatattaagacatataaagaacgagaac
attattatagcgttgctcgagtactaactggtacctcttcttttttttctgcagGTAGTAGTCGT
AGGTGATCAGAGTGCTGGAAAGACTAGTGTGTTGGAAATGATTGCCCAAGCTCGAATATTCCCAAGA
GGATCTGGGGAGATGATGACACGTTCTCCAGTTAAGGTGACTCTGAGTGAAGGTCCTCACCATGTGG
CCCTATTTAAAGATAGTTCTCGGGAGTTTGATCTTACCAAAGAAGAAGATCTTGCAGCATTAAGACA
TGAAATAGAACTTCGAATGAGGAAAAATGTGAAAGAAGGCTGTACCGTTAGCCCTGAGACCATATCC
TTAAATGTAAAAGGCCCTGGACTACAGAGGATGGTGCTTGTTGACTTACCAGGTGTGATTAATACTG
TGACATCAGGCATGGCTCCTGACACAAAGGAAACTATTTTCAGTATCAGCAAAGCTTACATGCAGAA
TCCTAATGCCATCATACTGTGTATTCAAGATGGATCTGTGGATGCTGAACGCAGTATTGTTACAGAC
TTGGTCAGTCAAATGGACCCTCATGGAAGGAGAACCATATTCGTTTTGACCAAAGTAGACCTGGCAG
AGAAAAATGTAGCCAGTCCAAGCAGGATTCAGCAGATAATTGAAGGAAAGCTCTTCCCAATGAAAGC
TTTAGGTTATTTTGCTGTTGTAACAGGAAAAGGGAACAGCTCTGAAAGCATTGAAGCTATAAGAGAA
TATGAAGAAGAGTTTTTTCAGAATTCAAAGCTCCTAAAGACAAGCATGCTAAAGGCACACCAAGTGA
CTACAAGAAATTTAAGCCTTGCAGTATCAGACTGCTTTTGGAAAATGGTACGAGAGTCTGTTGAACA
ACAGGCTGATAGTTTCAAAGCAACACGTTTTAACCTTGAAACTGAATGGAAGAATAACTATCCTCGC
CTGCGGGAACTTGACCGGAATGAACTATTTGAAAAAGCTAAAAATGAAATCCTTGATGAAGTTATCA
GTCTGAGCCAGGTTACACCAAAACATTGGGAGGAAATCCTTCAACAATCTTTGTGGGAAAGAGTATC
AACTCATGTGATTGAAAACATCTACCTTCCAGCTGCGCAGACCATGAATTCAGGAACTTTTAACACC
ACAGTGGATATCAAGCTTAAACAGTGGACTGATAAACAACTTCCTAATAAAGCAGTAGAGGTTGCTT
GGGAGACCCTACAAGAAGAATTTTCCCGCTTTATGACAGAACCGAAAGGGAAAGAGCATGATGACAT
ATTTGATAAACTTAAAGAGGCTGTTAAGGAAGAAAGTATTAAACGACACAAGTGGAATGACTTTGCG
GAGGACAGCTTGAGGGTTATTCAACACAATGCTTTGGAAGACCGATCCATATCTGATAAACAGCAAT
GGGATGCAGCTATTTATTTTATGGAAGAGGCTCTGCAGGCTCGTCTCAAGGATACTGAAAATGCAAT
TGAAAACATGGTGGGTCCAGACTGGAAAAAGAGGTGGTTATACTGGAAGAATCGGACCCAAGAACAG
TGTGTTCACAATGAAACCAAGAATGAATTGGAGAAGATGTTGAAATGTAATGAGGAGCACCCAGCTT
ATCTTGCAAGTGATGAAATAACCACAGTCCGGAAGAACCTTGAATCCCGAGGAGTAGAAGTAGATCC
AAGCTTGATTAAGGATACTTGGCATCAAGTTTATAGAAGACATTTTTTAAAAACAGCTCTAAACCAT
TGTAACCTTTGTCGAAGAGGTTTTTATTACTACCAAAGGCATTTTGTAGATTCTGAGTTGGAATGCA
ATGATGTGGTCTTGTTTTGGCGTATACAGCGCATGCTTGCTATCACCGCAAATACTTTAAGGCAACA
ACTTACAAATACTGAAGTTAGGCGATTAGAGAAAAATGTTAAAGAGGTATTGGAAGATTTTGCTGAA
GATGGTGAGAAGAAGATTAAATTGCTTACTGGTAAACGCGTTCAACTGGCGGAAGACCTCAAGAAAG
TTAGAGAAATTCAAGAAAAACTTGATGCTTTCATTGAAGCTCTTCATCAGGAGAAATAA;
-OPA1 RTMₜₕ#128:
(SEQ ID NO: 28)
cctaatttttattttaaaaacttgtgctaatttaaaccagtatttctaaaaccaggttattaac
tttccataaatcttttccatagaacgagaacattattatagcgttgctcgagtactaactggta
cctcttcttttttttctgcagGTAGTAGTCGTAGGTGATCAGAGTGCTGGAAAGACTAGTGTGTTG
GAAATGATTGCCCAAGCTCGAATATTCCCAAGAGGATCTGGGGAGATGATGACACGTTCTCCAGTTA
AGGTGACTCTGAGTGAAGGTCCTCACCATGTGGCCCTATTTAAAGATAGTTCTCGGGAGTTTGATCT
TACCAAAGAAGAAGATCTTGCAGCATTAAGACATGAAATAGAACTTCGAATGAGGAAAAATGTGAAA
GAAGGCTGTACCGTTAGCCCTGAGACCATATCCTTAAATGTAAAAGGCCCTGGACTACAGAGGATGG
TGCTTGTTGACTTACCAGGTGTGATTAATACTGTGACATCAGGCATGGCTCCTGACACAAAGGAAAC
TATTTTCAGTATCAGCAAAGCTTACATGCAGAATCCTAATGCCATCATACTGTGTATTCAAGATGGA
TCTGTGGATGCTGAACGCAGTATTGTTACAGACTTGGTCAGTCAAATGGACCCTCATGGAAGGAGAA
CCATATTCGTTTTGACCAAAGTAGACCTGGCAGAGAAAAATGTAGCCAGTCCAAGCAGGATTCAGCA
GATAATTGAAGGAAAGCTCTTCCCAATGAAAGCTTTAGGTTATTTTGCTGTTGTAACAGGAAAAGGG
AACAGCTCTGAAAGCATTGAAGCTATAAGAGAATATGAAGAAGAGTTTTTTCAGAATTCAAAGCTCC
TAAAGACAAGCATGCTAAAGGCACACCAAGTGACTACAAGAAATTTAAGCCTTGCAGTATCAGACTG
CTTTTGGAAAATGGTACGAGAGTCTGTTGAACAACAGGCTGATAGTTTCAAAGCAACACGTTTTAAC
CTTGAAACTGAATGGAAGAATAACTATCCTCGCCTGCGGGAACTTGACCGGAATGAACTATTTGAAA
AAGCTAAAAATGAAATCCTTGATGAAGTTATCAGTCTGAGCCAGGTTACACCAAAACATTGGGAGGA
AATCCTTCAACAATCTTTGTGGGAAAGAGTATCAACTCATGTGATTGAAAACATCTACCTTCCAGCT
GCGCAGACCATGAATTCAGGAACTTTTAACACCACAGTGGATATCAAGCTTAAACAGTGGACTGATA
AACAACTTCCTAATAAAGCAGTAGAGGTTGCTTGGGAGACCCTACAAGAAGAATTTTCCCGCTTTAT
GACAGAACCGAAAGGGAAAGAGCATGATGACATATTTGATAAACTTAAAGAGGCTGTTAAGGAAGAA
AGTATTAAACGACACAAGTGGAATGACTTTGCGGAGGACAGCTTGAGGGTTATTCAACACAATGCTT
TGGAAGACCGATCCATATCTGATAAACAGCAATGGGATGCAGCTATTTATTTTATGGAAGAGGCTCT
GCAGGCTCGTCTCAAGGATACTGAAAATGCAATTGAAAACATGGTGGGTCCAGACTGGAAAAAGAGG
TGGTTATACTGGAAGAATCGGACCCAAGAACAGTGTGTTCACAATGAAACCAAGAATGAATTGGAGA
AGATGTTGAAATGTAATGAGGAGCACCCAGCTTATCTTGCAAGTGATGAAATAACCACAGTCCGGAA
GAACCTTGAATCCCGAGGAGTAGAAGTAGATCCAAGCTTGATTAAGGATACTTGGCATCAAGTTTAT
AGAAGACATTTTTTAAAAACAGCTCTAAACCATTGTAACCTTTGTCGAAGAGGTTTTTATTACTACC
AAAGGCATTTTGTAGATTCTGAGTTGGAATGCAATGATGTGGTCTTGTTTTGGCGTATACAGCGCAT
GCTTGCTATCACCGCAAATACTTTAAGGCAACAACTTACAAATACTGAAGTTAGGCGATTAGAGAAA
AATGTTAAAGAGGTATTGGAAGATTTTGCTGAAGATGGTGAGAAGAAGATTAAATTGCTTACTGGTA
AACGCGTTCAACTGGCGGAAGACCTCAAGAAAGTTAGAGAAATTCAAGAAAAACTTGATGCTTTCAT
TGAAGCTCTTCATCAGGAGAAATAA;
-OPA1 RTMₜₕ#135:
(SEQ ID NO: 29)
ctgcttctttttttgctaattggcaagttcactatcaatttttcacatacctttatatgtctta
atattagtaatatttaaatactgaacagtggattaaattagaacgagaacattattatagcgtt
gctcgagtactaactggtacctcttcttttttttctgcagGTAGTAGTCGTAGGTGATCAGAGTG
CTGGAAAGACTAGTGTGTTGGAAATGATTGCCCAAGCTCGAATATTCCCAAGAGGATCTGGGGAGAT
GATGACACGTTCTCCAGTTAAGGTGACTCTGAGTGAAGGTCCTCACCATGTGGCCCTATTTAAAGAT
AGTTCTCGGGAGTTTGATCTTACCAAAGAAGAAGATCTTGCAGCATTAAGACATGAAATAGAACTTC
GAATGAGGAAAAATGTGAAAGAAGGCTGTACCGTTAGCCCTGAGACCATATCCTTAAATGTAAAAGG
CCCTGGACTACAGAGGATGGTGCTTGTTGACTTACCAGGTGTGATTAATACTGTGACATCAGGCATG
GCTCCTGACACAAAGGAAACTATTTTCAGTATCAGCAAAGCTTACATGCAGAATCCTAATGCCATCA
TACTGTGTATTCAAGATGGATCTGTGGATGCTGAACGCAGTATTGTTACAGACTTGGTCAGTCAAAT
GGACCCTCATGGAAGGAGAACCATATTCGTTTTGACCAAAGTAGACCTGGCAGAGAAAAATGTAGCC
AGTCCAAGCAGGATTCAGCAGATAATTGAAGGAAAGCTCTTCCCAATGAAAGCTTTAGGTTATTTTG
CTGTTGTAACAGGAAAAGGGAACAGCTCTGAAAGCATTGAAGCTATAAGAGAATATGAAGAAGAGTT
TTTTCAGAATTCAAAGCTCCTAAAGACAAGCATGCTAAAGGCACACCAAGTGACTACAAGAAATTTA
AGCCTTGCAGTATCAGACTGCTTTTGGAAAATGGTACGAGAGTCTGTTGAACAACAGGCTGATAGTT
TCAAAGCAACACGTTTTAACCTTGAAACTGAATGGAAGAATAACTATCCTCGCCTGCGGGAACTTGA
CCGGAATGAACTATTTGAAAAAGCTAAAAATGAAATCCTTGATGAAGTTATCAGTCTGAGCCAGGTT
ACACCAAAACATTGGGAGGAAATCCTTCAACAATCTTTGTGGGAAAGAGTATCAACTCATGTGATTG
AAAACATCTACCTTCCAGCTGCGCAGACCATGAATTCAGGAACTTTTAACACCACAGTGGATATCAA
GCTTAAACAGTGGACTGATAAACAACTTCCTAATAAAGCAGTAGAGGTTGCTTGGGAGACCCTACAA
GAAGAATTTTCCCGCTTTATGACAGAACCGAAAGGGAAAGAGCATGATGACATATTTGATAAACTTA
AAGAGGCTGTTAAGGAAGAAAGTATTAAACGACACAAGTGGAATGACTTTGCGGAGGACAGCTTGAG
GGTTATTCAACACAATGCTTTGGAAGACCGATCCATATCTGATAAACAGCAATGGGATGCAGCTATT
TATTTTATGGAAGAGGCTCTGCAGGCTCGTCTCAAGGATACTGAAAATGCAATTGAAAACATGGTGG
GTCCAGACTGGAAAAAGAGGTGGTTATACTGGAAGAATCGGACCCAAGAACAGTGTGTTCACAATGA
AACCAAGAATGAATTGGAGAAGATGTTGAAATGTAATGAGGAGCACCCAGCTTATCTTGCAAGTGAT
GAAATAACCACAGTCCGGAAGAACCTTGAATCCCGAGGAGTAGAAGTAGATCCAAGCTTGATTAAGG
ATACTTGGCATCAAGTTTATAGAAGACATTTTTTAAAAACAGCTCTAAACCATTGTAACCTTTGTCG
AAGAGGTTTTTATTACTACCAAAGGCATTTTGTAGATTCTGAGTTGGAATGCAATGATGTGGTCTTG
TTTTGGCGTATACAGCGCATGCTTGCTATCACCGCAAATACTTTAAGGCAACAACTTACAAATACTG
AAGTTAGGCGATTAGAGAAAAATGTTAAAGAGGTATTGGAAGATTTTGCTGAAGATGGTGAGAAGAA
GATTAAATTGCTTACTGGTAAACGCGTTCAACTGGCGGAAGACCTCAAGAAAGTTAGAGAAATTCAA
GAAAAACTTGATGCTTTCATTGAAGCTCTTCATCAGGAGAAATAA;
and
-OPA1 RTMₜₕ#162:
(SEQ ID NO: 30)
taatgctgctctgagttttttagtcttttaaaatttctacaggtgaaaaaatattttaaaatcc
gtaaaaaaatggtgattgatgcatgtgtgcacattgaaaataggaacagcaaaactataactgga
tccactgtttatagaacgagaacattattatagcgttgctcgagtactaactggtacctcttctt
ttttttctgcagGTAGTAGTCGTAGGTGATCAGAGTGCTGGAAAGACTAGTGTGTTGGAAATGATT
GCCCAAGCTCGAATATTCCCAAGAGGATCTGGGGAGATGATGACACGTTCTCCAGTTAAGGTGACTC
TGAGTGAAGGTCCTCACCATGTGGCCCTATTTAAAGATAGTTCTCGGGAGTTTGATCTTACCAAAGA
AGAAGATCTTGCAGCATTAAGACATGAAATAGAACTTCGAATGAGGAAAAATGTGAAAGAAGGCTGT
ACCGTTAGCCCTGAGACCATATCCTTAAATGTAAAAGGCCCTGGACTACAGAGGATGGTGCTTGTTG
ACTTACCAGGTGTGATTAATACTGTGACATCAGGCATGGCTCCTGACACAAAGGAAACTATTTTCAG
TATCAGCAAAGCTTACATGCAGAATCCTAATGCCATCATACTGTGTATTCAAGATGGATCTGTGGAT
GCTGAACGCAGTATTGTTACAGACTTGGTCAGTCAAATGGACCCTCATGGAAGGAGAACCATATTCG
TTTTGACCAAAGTAGACCTGGCAGAGAAAAATGTAGCCAGTCCAAGCAGGATTCAGCAGATAATTGA
AGGAAAGCTCTTCCCAATGAAAGCTTTAGGTTATTTTGCTGTTGTAACAGGAAAAGGGAACAGCTCT
GAAAGCATTGAAGCTATAAGAGAATATGAAGAAGAGTTTTTTCAGAATTCAAAGCTCCTAAAGACAA
GCATGCTAAAGGCACACCAAGTGACTACAAGAAATTTAAGCCTTGCAGTATCAGACTGCTTTTGGAA
AATGGTACGAGAGTCTGTTGAACAACAGGCTGATAGTTTCAAAGCAACACGTTTTAACCTTGAAACT
GAATGGAAGAATAACTATCCTCGCCTGCGGGAACTTGACCGGAATGAACTATTTGAAAAAGCTAAAA
ATGAAATCCTTGATGAAGTTATCAGTCTGAGCCAGGTTACACCAAAACATTGGGAGGAAATCCTTCA
ACAATCTTTGTGGGAAAGAGTATCAACTCATGTGATTGAAAACATCTACCTTCCAGCTGCGCAGACC
ATGAATTCAGGAACTTTTAACACCACAGTGGATATCAAGCTTAAACAGTGGACTGATAAACAACTTC
CTAATAAAGCAGTAGAGGTTGCTTGGGAGACCCTACAAGAAGAATTTTCCCGCTTTATGACAGAACC
GAAAGGGAAAGAGCATGATGACATATTTGATAAACTTAAAGAGGCTGTTAAGGAAGAAAGTATTAAA
CGACACAAGTGGAATGACTTTGCGGAGGACAGCTTGAGGGTTATTCAACACAATGCTTTGGAAGACC
GATCCATATCTGATAAACAGCAATGGGATGCAGCTATTTATTTTATGGAAGAGGCTCTGCAGGCTCG
TCTCAAGGATACTGAAAATGCAATTGAAAACATGGTGGGTCCAGACTGGAAAAAGAGGTGGTTATAC
TGGAAGAATCGGACCCAAGAACAGTGTGTTCACAATGAAACCAAGAATGAATTGGAGAAGATGTTGA
AATGTAATGAGGAGCACCCAGCTTATCTTGCAAGTGATGAAATAACCACAGTCCGGAAGAACCTTGA
ATCCCGAGGAGTAGAAGTAGATCCAAGCTTGATTAAGGATACTTGGCATCAAGTTTATAGAAGACAT
TTTTTAAAAACAGCTCTAAACCATTGTAACCTTTGTCGAAGAGGTTTTTATTACTACCAAAGGCATT
TTGTAGATTCTGAGTTGGAATGCAATGATGTGGTCTTGTTTTGGCGTATACAGCGCATGCTTGCTAT
CACCGCAAATACTTTAAGGCAACAACTTACAAATACTGAAGTTAGGCGATTAGAGAAAAATGTTAAA
GAGGTATTGGAAGATTTTGCTGAAGATGGTGAGAAGAAGATTAAATTGCTTACTGGTAAACGCGTTC
AACTGGCGGAAGACCTCAAGAAAGTTAGAGAAATTCAAGAAAAACTTGATGCTTTCATTGAAGCTCT
TCATCAGGAGAAATAA;
-OPA1 RTMₜₕ#100:
(SEQ ID NO: 31)
GCAATCATTTCCAACACACTAGTCTTTCCAGCACTCTGATCTCCAACCACAACAACCTTCCCAC
AAAACAAAGAACACTTATTCATATTTTTCTGATACCatcgatgttaacgagaacattattatag
cgttgctcgagtactaactggtacctcttcttttttttctgcagGTAGTAGTCGTAGGTGATCA
GAGTGCTGGAAAGACTAGTGTGTTGGAAATGATTGCCCAAGCTCGAATATTCCCAAGAGGATCT
GGGGAGATGATGACACGTTCTCCAGTTAAGGTGACTCTGAGTGAAGGTCCTCACCATGTGGCCC
TATTTAAAGATAGTTCTCGGGAGTTTGATCTTACCAAAGAAGAAGATCTTGCAGCATTAAGACA
TGAAATAGAACTTCGAATGAGGAAAAATGTGAAAGAAGGCTGTACCGTTAGCCCTGAGACCATA
TCCTTAAATGTAAAAGGCCCTGGACTACAGAGGATGGTGCTTGTTGACTTACCAGGTGTGATTA
ATACTGTGACATCAGGCATGGCTCCTGACACAAAGGAAACTATTTTCAGTATCAGCAAAGCTTA
CATGCAGAATCCTAATGCCATCATACTGTGTATTCAAGATGGATCTGTGGATGCTGAACGCAGT
ATTGTTACAGACTTGGTCAGTCAAATGGACCCTCATGGAAGGAGAACCATATTCGTTTTGACCA
AAGTAGACCTGGCAGAGAAAAATGTAGCCAGTCCAAGCAGGATTCAGCAGATAATTGAAGGAAA
GCTCTTCCCAATGAAAGCTTTAGGTTATTTTGCTGTTGTAACAGGAAAAGGGAACAGCTCTGAA
AGCATTGAAGCTATAAGAGAATATGAAGAAGAGTTTTTTCAGAATTCAAAGCTCCTAAAGACAA
GCATGCTAAAGGCACACCAAGTGACTACAAGAAATTTAAGCCTTGCAGTATCAGACTGCTTTTG
GAAAATGGTACGAGAGTCTGTTGAACAACAGGCTGATAGTTTCAAAGCAACACGTTTTAACCTT
GAAACTGAATGGAAGAATAACTATCCTCGCCTGCGGGAACTTGACCGGAATGAACTATTTGAAA
AAGCTAAAAATGAAATCCTTGATGAAGTTATCAGTCTGAGCCAGGTTACACCAAAACATTGGGA
GGAAATCCTTCAACAATCTTTGTGGGAAAGAGTATCAACTCATGTGATTGAAAACATCTACCTT
CCAGCTGCGCAGACCATGAATTCAGGAACTTTTAACACCACAGTGGATATCAAGCTTAAACAGT
GGACTGATAAACAACTTCCTAATAAAGCAGTAGAGGTTGCTTGGGAGACCCTACAAGAAGAATT
TTCCCGCTTTATGACAGAACCGAAAGGGAAAGAGCATGATGACATATTTGATAAACTTAAAGAG
GCTGTTAAGGAAGAAAGTATTAAACGACACAAGTGGAATGACTTTGCGGAGGACAGCTTGAGGG
TTATTCAACACAATGCTTTGGAAGACCGATCCATATCTGATAAACAGCAATGGGATGCAGCTAT
TTATTTTATGGAAGAGGCTCTGCAGGCTCGTCTCAAGGATACTGAAAATGCAATTGAAAACATG
GTGGGTCCAGACTGGAAAAAGAGGTGGTTATACTGGAAGAATCGGACCCAAGAACAGTGTGTTC
ACAATGAAACCAAGAATGAATTGGAGAAGATGTTGAAATGTAATGAGGAGCACCCAGCTTATCT
TGCAAGTGATGAAATAACCACAGTCCGGAAGAACCTTGAATCCCGAGGAGTAGAAGTAGATCCA
AGCTTGATTAAGGATACTTGGCATCAAGTTTATAGAAGACATTTTTTAAAAACAGCTCTAAACC
ATTGTAACCTTTGTCGAAGAGGTTTTTATTACTACCAAAGGCATTTTGTAGATTCTGAGTTGGA
ATGCAATGATGTGGTCTTGTTTTGGCGTATACAGCGCATGCTTGCTATCACCGCAAATACTTTA
AGGCAACAACTTACAAATACTGAAGTTAGGCGATTAGAGAAAAATGTTAAAGAGGTATTGGAAG
ATTTTGCTGAAGATGGTGAGAAGAAGATTAAATTGCTTACTGGTAAACGCGTTCAACTGGCGGA
AGACCTCAAGAAAGTTAGAGAAATTCAAGAAAAACTTGATGCTTTCATTGAAGCTCTTCATCAG
GAGAAATAA;
-OPA1 RTMₜₕ#: 130
(SEQ ID NO: 32)
CCAGATCCTCTTGGGAATATTCGAGCTTGGGCAATCATTTCCAACACACTAGTCTTTCCAGCAC
TCTGATCTCCAACCACAACAACCTTCCCACAAAACAAAGAACACTTATTCATATTTTTCTGATA
CCatcgatgttaacgagaacattattatagcgttgctcgagtactaactggtacctcttctttt
ttttctgcagGTAGTAGTCGTAGGTGATCAGAGTGCTGGAAAGACTAGTGTGTTGGAAATGATTGCC
CAAGCTCGAATATTCCCAAGAGGATCTGGGGAGATGATGACACGTTCTCCAGTTAAGGTGACTCTGA
GTGAAGGTCCTCACCATGTGGCCCTATTTAAAGATAGTTCTCGGGAGTTTGATCTTACCAAAGAAGA
AGATCTTGCAGCATTAAGACATGAAATAGAACTTCGAATGAGGAAAAATGTGAAAGAAGGCTGT
ACCGTTAGCCCTGAGACCATATCCTTAAATGTAAAAGGCCCTGGACTACAGAGGATGGTGCTTG
TTGACTTACCAGGTGTGATTAATACTGTGACATCAGGCATGGCTCCTGACACAAAGGAAACTAT
TTTCAGTATCAGCAAAGCTTACATGCAGAATCCTAATGCCATCATACTGTGTATTCAAGATGGA
TCTGTGGATGCTGAACGCAGTATTGTTACAGACTTGGTCAGTCAAATGGACCCTCATGGAAGGA
GAACCATATTCGTTTTGACCAAAGTAGACCTGGCAGAGAAAAATGTAGCCAGTCCAAGCAGGAT
TCAGCAGATAATTGAAGGAAAGCTCTTCCCAATGAAAGCTTTAGGTTATTTTGCTGTTGTAACA
GGAAAAGGGAACAGCTCTGAAAGCATTGAAGCTATAAGAGAATATGAAGAAGAGTTTTTTCAGA
ATTCAAAGCTCCTAAAGACAAGCATGCTAAAGGCACACCAAGTGACTACAAGAAATTTAAGCCT
TGCAGTATCAGACTGCTTTTGGAAAATGGTACGAGAGTCTGTTGAACAACAGGCTGATAGTTTC
AAAGCAACACGTTTTAACCTTGAAACTGAATGGAAGAATAACTATCCTCGCCTGCGGGAACTTG
ACCGGAATGAACTATTTGAAAAAGCTAAAAATGAAATCCTTGATGAAGTTATCAGTCTGAGCCA
GGTTACACCAAAACATTGGGAGGAAATCCTTCAACAATCTTTGTGGGAAAGAGTATCAACTCAT
GTGATTGAAAACATCTACCTTCCAGCTGCGCAGACCATGAATTCAGGAACTTTTAACACCACAG
TGGATATCAAGCTTAAACAGTGGACTGATAAACAACTTCCTAATAAAGCAGTAGAGGTTGCTTG
GGAGACCCTACAAGAAGAATTTTCCCGCTTTATGACAGAACCGAAAGGGAAAGAGCATGATGAC
ATATTTGATAAACTTAAAGAGGCTGTTAAGGAAGAAAGTATTAAACGACACAAGTGGAATGACT
TTGCGGAGGACAGCTTGAGGGTTATTCAACACAATGCTTTGGAAGACCGATCCATATCTGATAA
ACAGCAATGGGATGCAGCTATTTATTTTATGGAAGAGGCTCTGCAGGCTCGTCTCAAGGATACT
GAAAATGCAATTGAAAACATGGTGGGTCCAGACTGGAAAAAGAGGTGGTTATACTGGAAGAATC
GGACCCAAGAACAGTGTGTTCACAATGAAACCAAGAATGAATTGGAGAAGATGTTGAAATGTAA
TGAGGAGCACCCAGCTTATCTTGCAAGTGATGAAATAACCACAGTCCGGAAGAACCTTGAATCC
CGAGGAGTAGAAGTAGATCCAAGCTTGATTAAGGATACTTGGCATCAAGTTTATAGAAGACATT
TTTTAAAAACAGCTCTAAACCATTGTAACCTTTGTCGAAGAGGTTTTTATTACTACCAAAGGCA
TTTTGTAGATTCTGAGTTGGAATGCAATGATGTGGTCTTGTTTTGGCGTATACAGCGCATGCTT
GCTATCACCGCAAATACTTTAAGGCAACAACTTACAAATACTGAAGTTAGGCGATTAGAGAAAA
ATGTTAAAGAGGTATTGGAAGATTTTGCTGAAGATGGTGAGAAGAAGATTAAATTGCTTACTGG
TAAACGCGTTCAACTGGCGGAAGACCTCAAGAAAGTTAGAGAAATTCAAGAAAAACTTGATGCT
TTCATTGAAGCTCTTCATCAGGAGAAATAA;
-OPA1 RTMₜₕ#158:
(SEQ ID NO: 33)
CTTAACTGGAGAACGTGTCATCATCTCCCCAGATCCTCTTGGGAATATTCGAGCTTGGGCAATC
ATTTCCAACACACTAGTCTTTCCAGCACTCTGATCTCCAACCACAACAACCTTCCCACAAAACA
AAGAACACTTATTCATATTTTTCTGATACCatcgatgttaacgagaacattattatagcgttgc
tcgagtactaactggtacctcttcttttttttctgcagGTAGTAGTCGTAGGTGATCAGAGTGCT
GGAAAGACTAGTGTGTTGGAAATGATTGCCCAAGCTCGAATATTCCCAAGAGGATCTGGGGAGATGA
TGACACGTTCTCCAGTTAAGGTGACTCTGAGTGAAGGTCCTCACCATGTGGCCCTATTTAAAGATAG
TTCTCGGGAGTTTGATCTTACCAAAGAAGAAGATCTTGCAGCATTAAGACATGAAATAGAACTTC
GAATGAGGAAAAATGTGAAAGAAGGCTGTACCGTTAGCCCTGAGACCATATCCTTAAATGTAAA
AGGCCCTGGACTACAGAGGATGGTGCTTGTTGACTTACCAGGTGTGATTAATACTGTGACATCA
GGCATGGCTCCTGACACAAAGGAAACTATTTTCAGTATCAGCAAAGCTTACATGCAGAATCCTA
ATGCCATCATACTGTGTATTCAAGATGGATCTGTGGATGCTGAACGCAGTATTGTTACAGACTT
GGTCAGTCAAATGGACCCTCATGGAAGGAGAACCATATTCGTTTTGACCAAAGTAGACCTGGCA
GAGAAAAATGTAGCCAGTCCAAGCAGGATTCAGCAGATAATTGAAGGAAAGCTCTTCCCAATGA
AAGCTTTAGGTTATTTTGCTGTTGTAACAGGAAAAGGGAACAGCTCTGAAAGCATTGAAGCTAT
AAGAGAATATGAAGAAGAGTTTTTTCAGAATTCAAAGCTCCTAAAGACAAGCATGCTAAAGGCA
CACCAAGTGACTACAAGAAATTTAAGCCTTGCAGTATCAGACTGCTTTTGGAAAATGGTACGAG
AGTCTGTTGAACAACAGGCTGATAGTTTCAAAGCAACACGTTTTAACCTTGAAACTGAATGGAA
GAATAACTATCCTCGCCTGCGGGAACTTGACCGGAATGAACTATTTGAAAAAGCTAAAAATGAA
ATCCTTGATGAAGTTATCAGTCTGAGCCAGGTTACACCAAAACATTGGGAGGAAATCCTTCAAC
AATCTTTGTGGGAAAGAGTATCAACTCATGTGATTGAAAACATCTACCTTCCAGCTGCGCAGAC
CATGAATTCAGGAACTTTTAACACCACAGTGGATATCAAGCTTAAACAGTGGACTGATAAACAA
CTTCCTAATAAAGCAGTAGAGGTTGCTTGGGAGACCCTACAAGAAGAATTTTCCCGCTTTATGA
CAGAACCGAAAGGGAAAGAGCATGATGACATATTTGATAAACTTAAAGAGGCTGTTAAGGAAGA
AAGTATTAAACGACACAAGTGGAATGACTTTGCGGAGGACAGCTTGAGGGTTATTCAACACAAT
GCTTTGGAAGACCGATCCATATCTGATAAACAGCAATGGGATGCAGCTATTTATTTTATGGAAG
AGGCTCTGCAGGCTCGTCTCAAGGATACTGAAAATGCAATTGAAAACATGGTGGGTCCAGACTG
GAAAAAGAGGTGGTTATACTGGAAGAATCGGACCCAAGAACAGTGTGTTCACAATGAAACCAAG
AATGAATTGGAGAAGATGTTGAAATGTAATGAGGAGCACCCAGCTTATCTTGCAAGTGATGAAA
TAACCACAGTCCGGAAGAACCTTGAATCCCGAGGAGTAGAAGTAGATCCAAGCTTGATTAAGGA
TACTTGGCATCAAGTTTATAGAAGACATTTTTTAAAAACAGCTCTAAACCATTGTAACCTTTGT
CGAAGAGGTTTTTATTACTACCAAAGGCATTTTGTAGATTCTGAGTTGGAATGCAATGATGTGG
TCTTGTTTTGGCGTATACAGCGCATGCTTGCTATCACCGCAAATACTTTAAGGCAACAACTTAC
AAATACTGAAGTTAGGCGATTAGAGAAAAATGTTAAAGAGGTATTGGAAGATTTTGCTGAAGAT
GGTGAGAAGAAGATTAAATTGCTTACTGGTAAACGCGTTCAACTGGCGGAAGACCTCAAGAAAG
TTAGAGAAATTCAAGAAAAACTTGATGCTTTCATTGAAGCTCTTCATCAGGAGAAATAA;
-OPA1 RTMₜₕ#166:
(SEQ ID NO: 34)
GCAATCATTTCCAACACACTAGTCTTTCCAGCACTCTGATCTCCAACCACAACAACCTTCCCAC
AAAACAAAGAACACTTATTCATATTTTTCTGATACCAAATTAAAATTAAAACCTATTTGTAATG
CTGCTCTGAGTTTTTTAGTCTTTTAAAATTTCTACAGGatcgatgttaacgagaacattattat
agcgttgctcgagtactaactggtacctcttcttttttttctgcagGTAGTAGTCGTAGGTGATC
AGAGTGCTGGAAAGACTAGTGTGTTGGAAATGATTGCCCAAGCTCGAATATTCCCAAGAGGATCTGG
GGAGATGATGACACGTTCTCCAGTTAAGGTGACTCTGAGTGAAGGTCCTCACCATGTGGCCCTATTT
AAAGATAGTTCTCGGGAGTTTGATCTTACCAAAGAAGAAGATCTTGCAGCATTAAGACATGAAATA
GAACTTCGAATGAGGAAAAATGTGAAAGAAGGCTGTACCGTTAGCCCTGAGACCATATCCTTAA
ATGTAAAAGGCCCTGGACTACAGAGGATGGTGCTTGTTGACTTACCAGGTGTGATTAATACTGT
GACATCAGGCATGGCTCCTGACACAAAGGAAACTATTTTCAGTATCAGCAAAGCTTACATGCAG
AATCCTAATGCCATCATACTGTGTATTCAAGATGGATCTGTGGATGCTGAACGCAGTATTGTTA
CAGACTTGGTCAGTCAAATGGACCCTCATGGAAGGAGAACCATATTCGTTTTGACCAAAGTAGA
CCTGGCAGAGAAAAATGTAGCCAGTCCAAGCAGGATTCAGCAGATAATTGAAGGAAAGCTCTTC
CCAATGAAAGCTTTAGGTTATTTTGCTGTTGTAACAGGAAAAGGGAACAGCTCTGAAAGCATTG
AAGCTATAAGAGAATATGAAGAAGAGTTTTTTCAGAATTCAAAGCTCCTAAAGACAAGCATGCT
AAAGGCACACCAAGTGACTACAAGAAATTTAAGCCTTGCAGTATCAGACTGCTTTTGGAAAATG
GTACGAGAGTCTGTTGAACAACAGGCTGATAGTTTCAAAGCAACACGTTTTAACCTTGAAACTG
AATGGAAGAATAACTATCCTCGCCTGCGGGAACTTGACCGGAATGAACTATTTGAAAAAGCTAA
AAATGAAATCCTTGATGAAGTTATCAGTCTGAGCCAGGTTACACCAAAACATTGGGAGGAAATC
CTTCAACAATCTTTGTGGGAAAGAGTATCAACTCATGTGATTGAAAACATCTACCTTCCAGCTG
CGCAGACCATGAATTCAGGAACTTTTAACACCACAGTGGATATCAAGCTTAAACAGTGGACTGA
TAAACAACTTCCTAATAAAGCAGTAGAGGTTGCTTGGGAGACCCTACAAGAAGAATTTTCCCGC
TTTATGACAGAACCGAAAGGGAAAGAGCATGATGACATATTTGATAAACTTAAAGAGGCTGTTA
AGGAAGAAAGTATTAAACGACACAAGTGGAATGACTTTGCGGAGGACAGCTTGAGGGTTATTCA
ACACAATGCTTTGGAAGACCGATCCATATCTGATAAACAGCAATGGGATGCAGCTATTTATTTT
ATGGAAGAGGCTCTGCAGGCTCGTCTCAAGGATACTGAAAATGCAATTGAAAACATGGTGGGTC
CAGACTGGAAAAAGAGGTGGTTATACTGGAAGAATCGGACCCAAGAACAGTGTGTTCACAATGA
AACCAAGAATGAATTGGAGAAGATGTTGAAATGTAATGAGGAGCACCCAGCTTATCTTGCAAGT
GATGAAATAACCACAGTCCGGAAGAACCTTGAATCCCGAGGAGTAGAAGTAGATCCAAGCTTGA
TTAAGGATACTTGGCATCAAGTTTATAGAAGACATTTTTTAAAAACAGCTCTAAACCATTGTAA
CCTTTGTCGAAGAGGTTTTTATTACTACCAAAGGCATTTTGTAGATTCTGAGTTGGAATGCAAT
GATGTGGTCTTGTTTTGGCGTATACAGCGCATGCTTGCTATCACCGCAAATACTTTAAGGCAAC
AACTTACAAATACTGAAGTTAGGCGATTAGAGAAAAATGTTAAAGAGGTATTGGAAGATTTTGC
TGAAGATGGTGAGAAGAAGATTAAATTGCTTACTGGTAAACGCGTTCAACTGGCGGAAGACCTC
AAGAAAGTTAGAGAAATTCAAGAAAAACTTGATGCTTTCATTGAAGCTCTTCATCAGGAGAAAT
AA;
-OPA1 RTMₜₕ#196:
(SEQ ID NO: 35)
CCAGATCCTCTTGGGAATATTCGAGCTTGGGCAATCATTTCCAACACACTAGTCTTTCCAGCAC
TCTGATCTCCAACCACAACAACCTTCCCACAAAACAAAGAACACTTATTCATATTTTTCTGATA
CCAAATTAAAATTAAAACCTATTTGTAATGCTGCTCTGAGTTTTTTAGTCTTTTAAAATTTCTA
CAGGatcgatgttaacgagaacattattatagcgttgctcgagtactaactggtacctcttctt
ttttttctgcagGTAGTAGTCGTAGGTGATCAGAGTGCTGGAAAGACTAGTGTGTTGGAAATGATT
GCCCAAGCTCGAATATTCCCAAGAGGATCTGGGGAGATGATGACACGTTCTCCAGTTAAGGTGACTC
TGAGTGAAGGTCCTCACCATGTGGCCCTATTTAAAGATAGTTCTCGGGAGTTTGATCTTACCAAAGA
AGAAGATCTTGCAGCATTAAGACATGAAATAGAACTTCGAATGAGGAAAAATGTGAAAGAAGGC
TGTACCGTTAGCCCTGAGACCATATCCTTAAATGTAAAAGGCCCTGGACTACAGAGGATGGTGC
TTGTTGACTTACCAGGTGTGATTAATACTGTGACATCAGGCATGGCTCCTGACACAAAGGAAAC
TATTTTCAGTATCAGCAAAGCTTACATGCAGAATCCTAATGCCATCATACTGTGTATTCAAGAT
GGATCTGTGGATGCTGAACGCAGTATTGTTACAGACTTGGTCAGTCAAATGGACCCTCATGGAA
GGAGAACCATATTCGTTTTGACCAAAGTAGACCTGGCAGAGAAAAATGTAGCCAGTCCAAGCAG
GATTCAGCAGATAATTGAAGGAAAGCTCTTCCCAATGAAAGCTTTAGGTTATTTTGCTGTTGTA
ACAGGAAAAGGGAACAGCTCTGAAAGCATTGAAGCTATAAGAGAATATGAAGAAGAGTTTTTTC
AGAATTCAAAGCTCCTAAAGACAAGCATGCTAAAGGCACACCAAGTGACTACAAGAAATTTAAG
CCTTGCAGTATCAGACTGCTTTTGGAAAATGGTACGAGAGTCTGTTGAACAACAGGCTGATAGT
TTCAAAGCAACACGTTTTAACCTTGAAACTGAATGGAAGAATAACTATCCTCGCCTGCGGGAAC
TTGACCGGAATGAACTATTTGAAAAAGCTAAAAATGAAATCCTTGATGAAGTTATCAGTCTGAG
CCAGGTTACACCAAAACATTGGGAGGAAATCCTTCAACAATCTTTGTGGGAAAGAGTATCAACT
CATGTGATTGAAAACATCTACCTTCCAGCTGCGCAGACCATGAATTCAGGAACTTTTAACACCA
CAGTGGATATCAAGCTTAAACAGTGGACTGATAAACAACTTCCTAATAAAGCAGTAGAGGTTGC
TTGGGAGACCCTACAAGAAGAATTTTCCCGCTTTATGACAGAACCGAAAGGGAAAGAGCATGAT
GACATATTTGATAAACTTAAAGAGGCTGTTAAGGAAGAAAGTATTAAACGACACAAGTGGAATG
ACTTTGCGGAGGACAGCTTGAGGGTTATTCAACACAATGCTTTGGAAGACCGATCCATATCTGA
TAAACAGCAATGGGATGCAGCTATTTATTTTATGGAAGAGGCTCTGCAGGCTCGTCTCAAGGAT
ACTGAAAATGCAATTGAAAACATGGTGGGTCCAGACTGGAAAAAGAGGTGGTTATACTGGAAGA
ATCGGACCCAAGAACAGTGTGTTCACAATGAAACCAAGAATGAATTGGAGAAGATGTTGAAATG
TAATGAGGAGCACCCAGCTTATCTTGCAAGTGATGAAATAACCACAGTCCGGAAGAACCTTGAA
TCCCGAGGAGTAGAAGTAGATCCAAGCTTGATTAAGGATACTTGGCATCAAGTTTATAGAAGAC
ATTTTTTAAAAACAGCTCTAAACCATTGTAACCTTTGTCGAAGAGGTTTTTATTACTACCAAAG
GCATTTTGTAGATTCTGAGTTGGAATGCAATGATGTGGTCTTGTTTTGGCGTATACAGCGCATG
CTTGCTATCACCGCAAATACTTTAAGGCAACAACTTACAAATACTGAAGTTAGGCGATTAGAGA
AAAATGTTAAAGAGGTATTGGAAGATTTTGCTGAAGATGGTGAGAAGAAGATTAAATTGCTTAC
TGGTAAACGCGTTCAACTGGCGGAAGACCTCAAGAAAGTTAGAGAAATTCAAGAAAAACTTGAT
GCTTTCATTGAAGCTCTTCATCAGGAGAAATAA;
-OPA1 RTMₜₕ#224:
(SEQ ID NO: 36)
CTTAACTGGAGAACGTGTCATCATCTCCCCAGATCCTCTTGGGAATATTCGAGCTTGGGCAATC
ATTTCCAACACACTAGTCTTTCCAGCACTCTGATCTCCAACCACAACAACCTTCCCACAAAACA
AAGAACACTTATTCATATTTTTCTGATACCAAATTAAAATTAAAACCTATTTGTAATGCTGCTC
TGAGTTTTTTAGTCTTTTAAAATTTCTACAGGatcgatgttaacgagaacattattatagcgtt
gctcgagtactaactggtacctcttcttttttttctgcagGTAGTAGTCGTAGGTGATCAGAGTG
CTGGAAAGACTAGTGTGTTGGAAATGATTGCCCAAGCTCGAATATTCCCAAGAGGATCTGGGGAGAT
GATGACACGTTCTCCAGTTAAGGTGACTCTGAGTGAAGGTCCTCACCATGTGGCCCTATTTAAAGAT
AGTTCTCGGGAGTTTGATCTTACCAAAGAAGAAGATCTTGCAGCATTAAGACATGAAATAGAACT
TCGAATGAGGAAAAATGTGAAAGAAGGCTGTACCGTTAGCCCTGAGACCATATCCTTAAATGTA
AAAGGCCCTGGACTACAGAGGATGGTGCTTGTTGACTTACCAGGTGTGATTAATACTGTGACAT
CAGGCATGGCTCCTGACACAAAGGAAACTATTTTCAGTATCAGCAAAGCTTACATGCAGAATCC
TAATGCCATCATACTGTGTATTCAAGATGGATCTGTGGATGCTGAACGCAGTATTGTTACAGAC
TTGGTCAGTCAAATGGACCCTCATGGAAGGAGAACCATATTCGTTTTGACCAAAGTAGACCTGG
CAGAGAAAAATGTAGCCAGTCCAAGCAGGATTCAGCAGATAATTGAAGGAAAGCTCTTCCCAAT
GAAAGCTTTAGGTTATTTTGCTGTTGTAACAGGAAAAGGGAACAGCTCTGAAAGCATTGAAGCT
ATAAGAGAATATGAAGAAGAGTTTTTTCAGAATTCAAAGCTCCTAAAGACAAGCATGCTAAAGG
CACACCAAGTGACTACAAGAAATTTAAGCCTTGCAGTATCAGACTGCTTTTGGAAAATGGTACG
AGAGTCTGTTGAACAACAGGCTGATAGTTTCAAAGCAACACGTTTTAACCTTGAAACTGAATGG
AAGAATAACTATCCTCGCCTGCGGGAACTTGACCGGAATGAACTATTTGAAAAAGCTAAAAATG
AAATCCTTGATGAAGTTATCAGTCTGAGCCAGGTTACACCAAAACATTGGGAGGAAATCCTTCA
ACAATCTTTGTGGGAAAGAGTATCAACTCATGTGATTGAAAACATCTACCTTCCAGCTGCGCAG
ACCATGAATTCAGGAACTTTTAACACCACAGTGGATATCAAGCTTAAACAGTGGACTGATAAAC
AACTTCCTAATAAAGCAGTAGAGGTTGCTTGGGAGACCCTACAAGAAGAATTTTCCCGCTTTAT
GACAGAACCGAAAGGGAAAGAGCATGATGACATATTTGATAAACTTAAAGAGGCTGTTAAGGAA
GAAAGTATTAAACGACACAAGTGGAATGACTTTGCGGAGGACAGCTTGAGGGTTATTCAACACA
ATGCTTTGGAAGACCGATCCATATCTGATAAACAGCAATGGGATGCAGCTATTTATTTTATGGA
AGAGGCTCTGCAGGCTCGTCTCAAGGATACTGAAAATGCAATTGAAAACATGGTGGGTCCAGAC
TGGAAAAAGAGGTGGTTATACTGGAAGAATCGGACCCAAGAACAGTGTGTTCACAATGAAACCA
AGAATGAATTGGAGAAGATGTTGAAATGTAATGAGGAGCACCCAGCTTATCTTGCAAGTGATGA
AATAACCACAGTCCGGAAGAACCTTGAATCCCGAGGAGTAGAAGTAGATCCAAGCTTGATTAAG
GATACTTGGCATCAAGTTTATAGAAGACATTTTTTAAAAACAGCTCTAAACCATTGTAACCTTT
GTCGAAGAGGTTTTTATTACTACCAAAGGCATTTTGTAGATTCTGAGTTGGAATGCAATGATGT
GGTCTTGTTTTGGCGTATACAGCGCATGCTTGCTATCACCGCAAATACTTTAAGGCAACAACTT
ACAAATACTGAAGTTAGGCGATTAGAGAAAAATGTTAAAGAGGTATTGGAAGATTTTGCTGAAG
ATGGTGAGAAGAAGATTAAATTGCTTACTGGTAAACGCGTTCAACTGGCGGAAGACCTCAAGAA
AGTTAGAGAAATTCAAGAAAAACTTGATGCTTTCATTGAAGCTCTTCATCAGGAGAAATAA;
-OPA1 RTMₜₕ#209:
(SEQ ID NO: 37)
GCAATCATTTCCAACACACTAGTCTTTCCAGCACTCTGATCTCCAACCACAACAACCTTCCCAC
AAAACAAAGAACACTTATTCATATTTTTCTGATACCAAATTAAAATTAAAACCTATTTGTAATG
CTGCTCTGAGTTTTTTAGTCTTTTAAAATTTCTACAGGTGAAAAAATATTTTAAAATCCGTAAA
AAAATGGTGATTGATGCatcgatgttaacgagaacattattatagcgttgctcgagtactaact
ggtacctcttcttttttttctgcagGTAGTAGTCGTAGGTGATCAGAGTGCTGGAAAGACTAGTGT
GTTGGAAATGATTGCCCAAGCTCGAATATTCCCAAGAGGATCTGGGGAGATGATGACACGTTCTCCA
GTTAAGGTGACTCTGAGTGAAGGTCCTCACCATGTGGCCCTATTTAAAGATAGTTCTCGGGAGTTTG
ATCTTACCAAAGAAGAAGATCTTGCAGCATTAAGACATGAAATAGAACTTCGAATGAGGAAAAAT
GTGAAAGAAGGCTGTACCGTTAGCCCTGAGACCATATCCTTAAATGTAAAAGGCCCTGGACTAC
AGAGGATGGTGCTTGTTGACTTACCAGGTGTGATTAATACTGTGACATCAGGCATGGCTCCTGA
CACAAAGGAAACTATTTTCAGTATCAGCAAAGCTTACATGCAGAATCCTAATGCCATCATACTG
TGTATTCAAGATGGATCTGTGGATGCTGAACGCAGTATTGTTACAGACTTGGTCAGTCAAATGG
ACCCTCATGGAAGGAGAACCATATTCGTTTTGACCAAAGTAGACCTGGCAGAGAAAAATGTAGC
CAGTCCAAGCAGGATTCAGCAGATAATTGAAGGAAAGCTCTTCCCAATGAAAGCTTTAGGTTAT
TTTGCTGTTGTAACAGGAAAAGGGAACAGCTCTGAAAGCATTGAAGCTATAAGAGAATATGAAG
AAGAGTTTTTTCAGAATTCAAAGCTCCTAAAGACAAGCATGCTAAAGGCACACCAAGTGACTAC
AAGAAATTTAAGCCTTGCAGTATCAGACTGCTTTTGGAAAATGGTACGAGAGTCTGTTGAACAA
CAGGCTGATAGTTTCAAAGCAACACGTTTTAACCTTGAAACTGAATGGAAGAATAACTATCCTC
GCCTGCGGGAACTTGACCGGAATGAACTATTTGAAAAAGCTAAAAATGAAATCCTTGATGAAGT
TATCAGTCTGAGCCAGGTTACACCAAAACATTGGGAGGAAATCCTTCAACAATCTTTGTGGGAA
AGAGTATCAACTCATGTGATTGAAAACATCTACCTTCCAGCTGCGCAGACCATGAATTCAGGAA
CTTTTAACACCACAGTGGATATCAAGCTTAAACAGTGGACTGATAAACAACTTCCTAATAAAGC
AGTAGAGGTTGCTTGGGAGACCCTACAAGAAGAATTTTCCCGCTTTATGACAGAACCGAAAGGG
AAAGAGCATGATGACATATTTGATAAACTTAAAGAGGCTGTTAAGGAAGAAAGTATTAAACGAC
ACAAGTGGAATGACTTTGCGGAGGACAGCTTGAGGGTTATTCAACACAATGCTTTGGAAGACCG
ATCCATATCTGATAAACAGCAATGGGATGCAGCTATTTATTTTATGGAAGAGGCTCTGCAGGCT
CGTCTCAAGGATACTGAAAATGCAATTGAAAACATGGTGGGTCCAGACTGGAAAAAGAGGTGGT
TATACTGGAAGAATCGGACCCAAGAACAGTGTGTTCACAATGAAACCAAGAATGAATTGGAGAA
GATGTTGAAATGTAATGAGGAGCACCCAGCTTATCTTGCAAGTGATGAAATAACCACAGTCCGG
AAGAACCTTGAATCCCGAGGAGTAGAAGTAGATCCAAGCTTGATTAAGGATACTTGGCATCAAG
TTTATAGAAGACATTTTTTAAAAACAGCTCTAAACCATTGTAACCTTTGTCGAAGAGGTTTTTA
TTACTACCAAAGGCATTTTGTAGATTCTGAGTTGGAATGCAATGATGTGGTCTTGTTTTGGCGT
ATACAGCGCATGCTTGCTATCACCGCAAATACTTTAAGGCAACAACTTACAAATACTGAAGTTA
GGCGATTAGAGAAAAATGTTAAAGAGGTATTGGAAGATTTTGCTGAAGATGGTGAGAAGAAGAT
TAAATTGCTTACTGGTAAACGCGTTCAACTGGCGGAAGACCTCAAGAAAGTTAGAGAAATTCAA
GAAAAACTTGATGCTTTCATTGAAGCTCTTCATCAGGAGAAATAA;
-OPA1 RTMₜₕ#239:
(SEQ ID NO: 38)
CCAGATCCTCTTGGGAATATTCGAGCTTGGGCAATCATTTCCAACACACTAGTCTTTCCAGCAC
TCTGATCTCCAACCACAACAACCTTCCCACAAAACAAAGAACACTTATTCATATTTTTCTGATA
CCAAATTAAAATTAAAACCTATTTGTAATGCTGCTCTGAGTTTTTTAGTCTTTTAAAATTTCTA
CAGGTGAAAAAATATTTTAAAATCCGTAAAAAAATGGTGATTGATGCatcgatgttaacgagaa
cattattatagcgttgctcgagtactaactggtacctcttcttttttttctgcagGTAGTAGTCG
TAGGTGATCAGAGTGCTGGAAAGACTAGTGTGTTGGAAATGATTGCCCAAGCTCGAATATTCCCAAG
AGGATCTGGGGAGATGATGACACGTTCTCCAGTTAAGGTGACTCTGAGTGAAGGTCCTCACCATGTG
GCCCTATTTAAAGATAGTTCTCGGGAGTTTGATCTTACCAAAGAAGAAGATCTTGCAGCATTAAGA
CATGAAATAGAACTTCGAATGAGGAAAAATGTGAAAGAAGGCTGTACCGTTAGCCCTGAGACCA
TATCCTTAAATGTAAAAGGCCCTGGACTACAGAGGATGGTGCTTGTTGACTTACCAGGTGTGAT
TAATACTGTGACATCAGGCATGGCTCCTGACACAAAGGAAACTATTTTCAGTATCAGCAAAGCT
TACATGCAGAATCCTAATGCCATCATACTGTGTATTCAAGATGGATCTGTGGATGCTGAACGCA
GTATTGTTACAGACTTGGTCAGTCAAATGGACCCTCATGGAAGGAGAACCATATTCGTTTTGAC
CAAAGTAGACCTGGCAGAGAAAAATGTAGCCAGTCCAAGCAGGATTCAGCAGATAATTGAAGGA
AAGCTCTTCCCAATGAAAGCTTTAGGTTATTTTGCTGTTGTAACAGGAAAAGGGAACAGCTCTG
AAAGCATTGAAGCTATAAGAGAATATGAAGAAGAGTTTTTTCAGAATTCAAAGCTCCTAAAGAC
AAGCATGCTAAAGGCACACCAAGTGACTACAAGAAATTTAAGCCTTGCAGTATCAGACTGCTTT
TGGAAAATGGTACGAGAGTCTGTTGAACAACAGGCTGATAGTTTCAAAGCAACACGTTTTAACC
TTGAAACTGAATGGAAGAATAACTATCCTCGCCTGCGGGAACTTGACCGGAATGAACTATTTGA
AAAAGCTAAAAATGAAATCCTTGATGAAGTTATCAGTCTGAGCCAGGTTACACCAAAACATTGG
GAGGAAATCCTTCAACAATCTTTGTGGGAAAGAGTATCAACTCATGTGATTGAAAACATCTACC
TTCCAGCTGCGCAGACCATGAATTCAGGAACTTTTAACACCACAGTGGATATCAAGCTTAAACA
GTGGACTGATAAACAACTTCCTAATAAAGCAGTAGAGGTTGCTTGGGAGACCCTACAAGAAGAA
TTTTCCCGCTTTATGACAGAACCGAAAGGGAAAGAGCATGATGACATATTTGATAAACTTAAAG
AGGCTGTTAAGGAAGAAAGTATTAAACGACACAAGTGGAATGACTTTGCGGAGGACAGCTTGAG
GGTTATTCAACACAATGCTTTGGAAGACCGATCCATATCTGATAAACAGCAATGGGATGCAGCT
ATTTATTTTATGGAAGAGGCTCTGCAGGCTCGTCTCAAGGATACTGAAAATGCAATTGAAAACA
TGGTGGGTCCAGACTGGAAAAAGAGGTGGTTATACTGGAAGAATCGGACCCAAGAACAGTGTGT
TCACAATGAAACCAAGAATGAATTGGAGAAGATGTTGAAATGTAATGAGGAGCACCCAGCTTAT
CTTGCAAGTGATGAAATAACCACAGTCCGGAAGAACCTTGAATCCCGAGGAGTAGAAGTAGATC
CAAGCTTGATTAAGGATACTTGGCATCAAGTTTATAGAAGACATTTTTTAAAAACAGCTCTAAA
CCATTGTAACCTTTGTCGAAGAGGTTTTTATTACTACCAAAGGCATTTTGTAGATTCTGAGTTG
GAATGCAATGATGTGGTCTTGTTTTGGCGTATACAGCGCATGCTTGCTATCACCGCAAATACTT
TAAGGCAACAACTTACAAATACTGAAGTTAGGCGATTAGAGAAAAATGTTAAAGAGGTATTGGA
AGATTTTGCTGAAGATGGTGAGAAGAAGATTAAATTGCTTACTGGTAAACGCGTTCAACTGGCG
GAAGACCTCAAGAAAGTTAGAGAAATTCAAGAAAAACTTGATGCTTTCATTGAAGCTCTTCATC
AGGAGAAATAA;
-OPA1 RTMₜₕ#267:
(SEQ ID NO: 39)
CTTAACTGGAGAACGTGTCATCATCTCCCCAGATCCTCTTGGGAATATTCGAGCTTGGGCAATC
ATTTCCAACACACTAGTCTTTCCAGCACTCTGATCTCCAACCACAACAACCTTCCCACAAAACA
AAGAACACTTATTCATATTTTTCTGATACCAAATTAAAATTAAAACCTATTTGTAATGCTGCTC
TGAGTTTTTTAGTCTTTTAAAATTTCTACAGGTGAAAAAATATTTTAAAATCCGTAAAAAAATG
GTGATTGATGCatcgatgttaacgagaacattattatagcgttgctcgagtactaactggtacc
tcttcttttttttctgcagGTAGTAGTCGTAGGTGATCAGAGTGCTGGAAAGACTAGTGTGTTGGA
AATGATTGCCCAAGCTCGAATATTCCCAAGAGGATCTGGGGAGATGATGACACGTTCTCCAGTTAAG
GTGACTCTGAGTGAAGGTCCTCACCATGTGGCCCTATTTAAAGATAGTTCTCGGGAGTTTGATCTTA
CCAAAGAAGAAGATCTTGCAGCATTAAGACATGAAATAGAACTTCGAATGAGGAAAAATGTGAA
AGAAGGCTGTACCGTTAGCCCTGAGACCATATCCTTAAATGTAAAAGGCCCTGGACTACAGAGG
ATGGTGCTTGTTGACTTACCAGGTGTGATTAATACTGTGACATCAGGCATGGCTCCTGACACAA
AGGAAACTATTTTCAGTATCAGCAAAGCTTACATGCAGAATCCTAATGCCATCATACTGTGTAT
TCAAGATGGATCTGTGGATGCTGAACGCAGTATTGTTACAGACTTGGTCAGTCAAATGGACCCT
CATGGAAGGAGAACCATATTCGTTTTGACCAAAGTAGACCTGGCAGAGAAAAATGTAGCCAGTC
CAAGCAGGATTCAGCAGATAATTGAAGGAAAGCTCTTCCCAATGAAAGCTTTAGGTTATTTTGC
TGTTGTAACAGGAAAAGGGAACAGCTCTGAAAGCATTGAAGCTATAAGAGAATATGAAGAAGAG
TTTTTTCAGAATTCAAAGCTCCTAAAGACAAGCATGCTAAAGGCACACCAAGTGACTACAAGAA
ATTTAAGCCTTGCAGTATCAGACTGCTTTTGGAAAATGGTACGAGAGTCTGTTGAACAACAGGC
TGATAGTTTCAAAGCAACACGTTTTAACCTTGAAACTGAATGGAAGAATAACTATCCTCGCCTG
CGGGAACTTGACCGGAATGAACTATTTGAAAAAGCTAAAAATGAAATCCTTGATGAAGTTATCA
GTCTGAGCCAGGTTACACCAAAACATTGGGAGGAAATCCTTCAACAATCTTTGTGGGAAAGAGT
ATCAACTCATGTGATTGAAAACATCTACCTTCCAGCTGCGCAGACCATGAATTCAGGAACTTTT
AACACCACAGTGGATATCAAGCTTAAACAGTGGACTGATAAACAACTTCCTAATAAAGCAGTAG
AGGTTGCTTGGGAGACCCTACAAGAAGAATTTTCCCGCTTTATGACAGAACCGAAAGGGAAAGA
GCATGATGACATATTTGATAAACTTAAAGAGGCTGTTAAGGAAGAAAGTATTAAACGACACAAG
TGGAATGACTTTGCGGAGGACAGCTTGAGGGTTATTCAACACAATGCTTTGGAAGACCGATCCA
TATCTGATAAACAGCAATGGGATGCAGCTATTTATTTTATGGAAGAGGCTCTGCAGGCTCGTCT
CAAGGATACTGAAAATGCAATTGAAAACATGGTGGGTCCAGACTGGAAAAAGAGGTGGTTATAC
TGGAAGAATCGGACCCAAGAACAGTGTGTTCACAATGAAACCAAGAATGAATTGGAGAAGATGT
TGAAATGTAATGAGGAGCACCCAGCTTATCTTGCAAGTGATGAAATAACCACAGTCCGGAAGAA
CCTTGAATCCCGAGGAGTAGAAGTAGATCCAAGCTTGATTAAGGATACTTGGCATCAAGTTTAT
AGAAGACATTTTTTAAAAACAGCTCTAAACCATTGTAACCTTTGTCGAAGAGGTTTTTATTACT
ACCAAAGGCATTTTGTAGATTCTGAGTTGGAATGCAATGATGTGGTCTTGTTTTGGCGTATACA
GCGCATGCTTGCTATCACCGCAAATACTTTAAGGCAACAACTTACAAATACTGAAGTTAGGCGA
TTAGAGAAAAATGTTAAAGAGGTATTGGAAGATTTTGCTGAAGATGGTGAGAAGAAGATTAAAT
TGCTTACTGGTAAACGCGTTCAACTGGCGGAAGACCTCAAGAAAGTTAGAGAAATTCAAGAAAA
ACTTGATGCTTTCATTGAAGCTCTTCATCAGGAGAAATAA;
-OPA1 RTMₜₕ#217:
(SEQ ID NO: 40)
GCAATCATTTCCAACACACTAGTCTTTCCAGCACTCTGATCTCCAACCACAACAACCTTCCCAC
AAAACAAAGAACACTTATTCATATTTTTCTGATACCAAATTAAAATTAAAACCTATTTGTAATG
CTGCTCTGAGTTTTTTAGTCTTTTAAAATTTCTACAGGTGAAAAAATATTTTAAAATCCGTAAA
AAAATGGTGATTGATGCATGTGTGCatcgatgttaacgagaacattattatagcgttgctcgag
tactaactggtacctcttcttttttttctgcagGTAGTAGTCGTAGGTGATCAGAGTGCTGGAAAG
ACTAGTGTGTTGGAAATGATTGCCCAAGCTCGAATATTCCCAAGAGGATCTGGGGAGATGATGACAC
GTTCTCCAGTTAAGGTGACTCTGAGTGAAGGTCCTCACCATGTGGCCCTATTTAAAGATAGTTCTCG
GGAGTTTGATCTTACCAAAGAAGAAGATCTTGCAGCATTAAGACATGAAATAGAACTTCGAATGA
GGAAAAATGTGAAAGAAGGCTGTACCGTTAGCCCTGAGACCATATCCTTAAATGTAAAAGGCCC
TGGACTACAGAGGATGGTGCTTGTTGACTTACCAGGTGTGATTAATACTGTGACATCAGGCATG
GCTCCTGACACAAAGGAAACTATTTTCAGTATCAGCAAAGCTTACATGCAGAATCCTAATGCCA
TCATACTGTGTATTCAAGATGGATCTGTGGATGCTGAACGCAGTATTGTTACAGACTTGGTCAG
TCAAATGGACCCTCATGGAAGGAGAACCATATTCGTTTTGACCAAAGTAGACCTGGCAGAGAAA
AATGTAGCCAGTCCAAGCAGGATTCAGCAGATAATTGAAGGAAAGCTCTTCCCAATGAAAGCTT
TAGGTTATTTTGCTGTTGTAACAGGAAAAGGGAACAGCTCTGAAAGCATTGAAGCTATAAGAGA
ATATGAAGAAGAGTTTTTTCAGAATTCAAAGCTCCTAAAGACAAGCATGCTAAAGGCACACCAA
GTGACTACAAGAAATTTAAGCCTTGCAGTATCAGACTGCTTTTGGAAAATGGTACGAGAGTCTG
TTGAACAACAGGCTGATAGTTTCAAAGCAACACGTTTTAACCTTGAAACTGAATGGAAGAATAA
CTATCCTCGCCTGCGGGAACTTGACCGGAATGAACTATTTGAAAAAGCTAAAAATGAAATCCTT
GATGAAGTTATCAGTCTGAGCCAGGTTACACCAAAACATTGGGAGGAAATCCTTCAACAATCTT
TGTGGGAAAGAGTATCAACTCATGTGATTGAAAACATCTACCTTCCAGCTGCGCAGACCATGAA
TTCAGGAACTTTTAACACCACAGTGGATATCAAGCTTAAACAGTGGACTGATAAACAACTTCCT
AATAAAGCAGTAGAGGTTGCTTGGGAGACCCTACAAGAAGAATTTTCCCGCTTTATGACAGAAC
CGAAAGGGAAAGAGCATGATGACATATTTGATAAACTTAAAGAGGCTGTTAAGGAAGAAAGTAT
TAAACGACACAAGTGGAATGACTTTGCGGAGGACAGCTTGAGGGTTATTCAACACAATGCTTTG
GAAGACCGATCCATATCTGATAAACAGCAATGGGATGCAGCTATTTATTTTATGGAAGAGGCTC
TGCAGGCTCGTCTCAAGGATACTGAAAATGCAATTGAAAACATGGTGGGTCCAGACTGGAAAAA
GAGGTGGTTATACTGGAAGAATCGGACCCAAGAACAGTGTGTTCACAATGAAACCAAGAATGAA
TTGGAGAAGATGTTGAAATGTAATGAGGAGCACCCAGCTTATCTTGCAAGTGATGAAATAACCA
CAGTCCGGAAGAACCTTGAATCCCGAGGAGTAGAAGTAGATCCAAGCTTGATTAAGGATACTTG
GCATCAAGTTTATAGAAGACATTTTTTAAAAACAGCTCTAAACCATTGTAACCTTTGTCGAAGA
GGTTTTTATTACTACCAAAGGCATTTTGTAGATTCTGAGTTGGAATGCAATGATGTGGTCTTGT
TTTGGCGTATACAGCGCATGCTTGCTATCACCGCAAATACTTTAAGGCAACAACTTACAAATAC
TGAAGTTAGGCGATTAGAGAAAAATGTTAAAGAGGTATTGGAAGATTTTGCTGAAGATGGTGAG
AAGAAGATTAAATTGCTTACTGGTAAACGCGTTCAACTGGCGGAAGACCTCAAGAAAGTTAGAG
AAATTCAAGAAAAACTTGATGCTTTCATTGAAGCTCTTCATCAGGAGAAATAA;
-OPA1 RTMₜₕ#247:
(SEQ ID NO: 41)
CCAGATCCTCTTGGGAATATTCGAGCTTGGGCAATCATTTCCAACACACTAGTCTTTCCAGCAC
TCTGATCTCCAACCACAACAACCTTCCCACAAAACAAAGAACACTTATTCATATTTTTCTGATA
CCAAATTAAAATTAAAACCTATTTGTAATGCTGCTCTGAGTTTTTTAGTCTTTTAAAATTTCTA
CAGGTGAAAAAATATTTTAAAATCCGTAAAAAAATGGTGATTGATGCATGTGTGCatcgatgtt
aacgagaacattattatagcgttgctcgagtactaactggtacctcttcttttttttctgcagG
TAGTAGTCGTAGGTGATCAGAGTGCTGGAAAGACTAGTGTGTTGGAAATGATTGCCCAAGCTCGAAT
ATTCCCAAGAGGATCTGGGGAGATGATGACACGTTCTCCAGTTAAGGTGACTCTGAGTGAAGGTCCT
CACCATGTGGCCCTATTTAAAGATAGTTCTCGGGAGTTTGATCTTACCAAAGAAGAAGATCTTGCA
GCATTAAGACATGAAATAGAACTTCGAATGAGGAAAAATGTGAAAGAAGGCTGTACCGTTAGCC
CTGAGACCATATCCTTAAATGTAAAAGGCCCTGGACTACAGAGGATGGTGCTTGTTGACTTACC
AGGTGTGATTAATACTGTGACATCAGGCATGGCTCCTGACACAAAGGAAACTATTTTCAGTATC
AGCAAAGCTTACATGCAGAATCCTAATGCCATCATACTGTGTATTCAAGATGGATCTGTGGATG
CTGAACGCAGTATTGTTACAGACTTGGTCAGTCAAATGGACCCTCATGGAAGGAGAACCATATT
CGTTTTGACCAAAGTAGACCTGGCAGAGAAAAATGTAGCCAGTCCAAGCAGGATTCAGCAGATA
ATTGAAGGAAAGCTCTTCCCAATGAAAGCTTTAGGTTATTTTGCTGTTGTAACAGGAAAAGGGA
ACAGCTCTGAAAGCATTGAAGCTATAAGAGAATATGAAGAAGAGTTTTTTCAGAATTCAAAGCT
CCTAAAGACAAGCATGCTAAAGGCACACCAAGTGACTACAAGAAATTTAAGCCTTGCAGTATCA
GACTGCTTTTGGAAAATGGTACGAGAGTCTGTTGAACAACAGGCTGATAGTTTCAAAGCAACAC
GTTTTAACCTTGAAACTGAATGGAAGAATAACTATCCTCGCCTGCGGGAACTTGACCGGAATGA
ACTATTTGAAAAAGCTAAAAATGAAATCCTTGATGAAGTTATCAGTCTGAGCCAGGTTACACCA
AAACATTGGGAGGAAATCCTTCAACAATCTTTGTGGGAAAGAGTATCAACTCATGTGATTGAAA
ACATCTACCTTCCAGCTGCGCAGACCATGAATTCAGGAACTTTTAACACCACAGTGGATATCAA
GCTTAAACAGTGGACTGATAAACAACTTCCTAATAAAGCAGTAGAGGTTGCTTGGGAGACCCTA
CAAGAAGAATTTTCCCGCTTTATGACAGAACCGAAAGGGAAAGAGCATGATGACATATTTGATA
AACTTAAAGAGGCTGTTAAGGAAGAAAGTATTAAACGACACAAGTGGAATGACTTTGCGGAGGA
CAGCTTGAGGGTTATTCAACACAATGCTTTGGAAGACCGATCCATATCTGATAAACAGCAATGG
GATGCAGCTATTTATTTTATGGAAGAGGCTCTGCAGGCTCGTCTCAAGGATACTGAAAATGCAA
TTGAAAACATGGTGGGTCCAGACTGGAAAAAGAGGTGGTTATACTGGAAGAATCGGACCCAAGA
ACAGTGTGTTCACAATGAAACCAAGAATGAATTGGAGAAGATGTTGAAATGTAATGAGGAGCAC
CCAGCTTATCTTGCAAGTGATGAAATAACCACAGTCCGGAAGAACCTTGAATCCCGAGGAGTAG
AAGTAGATCCAAGCTTGATTAAGGATACTTGGCATCAAGTTTATAGAAGACATTTTTTAAAAAC
AGCTCTAAACCATTGTAACCTTTGTCGAAGAGGTTTTTATTACTACCAAAGGCATTTTGTAGAT
TCTGAGTTGGAATGCAATGATGTGGTCTTGTTTTGGCGTATACAGCGCATGCTTGCTATCACCG
CAAATACTTTAAGGCAACAACTTACAAATACTGAAGTTAGGCGATTAGAGAAAAATGTTAAAGA
GGTATTGGAAGATTTTGCTGAAGATGGTGAGAAGAAGATTAAATTGCTTACTGGTAAACGCGTT
CAACTGGCGGAAGACCTCAAGAAAGTTAGAGAAATTCAAGAAAAACTTGATGCTTTCATTGAAG
CTCTTCATCAGGAGAAATAA;
and
-OPA1 RTMₜₕ#275:
(SEQ ID NO: 42)
CTTAACTGGAGAACGTGTCATCATCTCCCCAGATCCTCTTGGGAATATTCGAGCTTGGGCAATC
ATTTCCAACACACTAGTCTTTCCAGCACTCTGATCTCCAACCACAACAACCTTCCCACAAAACA
AAGAACACTTATTCATATTTTTCTGATACCAAATTAAAATTAAAACCTATTTGTAATGCTGCTC
TGAGTTTTTTAGTCTTTTAAAATTTCTACAGGTGAAAAAATATTTTAAAATCCGTAAAAAAATG
GTGATTGATGCATGTGTGCatcgatgttaacgagaacattattatagcgttgctcgagtactaa
ctggtacctcttcttttttttctgcagGTAGTAGTCGTAGGTGATCAGAGTGCTGGAAAGACTAGT
GTGTTGGAAATGATTGCCCAAGCTCGAATATTCCCAAGAGGATCTGGGGAGATGATGACACGTTCTC
CAGTTAAGGTGACTCTGAGTGAAGGTCCTCACCATGTGGCCCTATTTAAAGATAGTTCTCGGGAGTT
TGATCTTACCAAAGAAGAAGATCTTGCAGCATTAAGACATGAAATAGAACTTCGAATGAGGAAAA
ATGTGAAAGAAGGCTGTACCGTTAGCCCTGAGACCATATCCTTAAATGTAAAAGGCCCTGGACT
ACAGAGGATGGTGCTTGTTGACTTACCAGGTGTGATTAATACTGTGACATCAGGCATGGCTCCT
GACACAAAGGAAACTATTTTCAGTATCAGCAAAGCTTACATGCAGAATCCTAATGCCATCATAC
TGTGTATTCAAGATGGATCTGTGGATGCTGAACGCAGTATTGTTACAGACTTGGTCAGTCAAAT
GGACCCTCATGGAAGGAGAACCATATTCGTTTTGACCAAAGTAGACCTGGCAGAGAAAAATGTA
GCCAGTCCAAGCAGGATTCAGCAGATAATTGAAGGAAAGCTCTTCCCAATGAAAGCTTTAGGTT
ATTTTGCTGTTGTAACAGGAAAAGGGAACAGCTCTGAAAGCATTGAAGCTATAAGAGAATATGA
AGAAGAGTTTTTTCAGAATTCAAAGCTCCTAAAGACAAGCATGCTAAAGGCACACCAAGTGACT
ACAAGAAATTTAAGCCTTGCAGTATCAGACTGCTTTTGGAAAATGGTACGAGAGTCTGTTGAAC
AACAGGCTGATAGTTTCAAAGCAACACGTTTTAACCTTGAAACTGAATGGAAGAATAACTATCC
TCGCCTGCGGGAACTTGACCGGAATGAACTATTTGAAAAAGCTAAAAATGAAATCCTTGATGAA
GTTATCAGTCTGAGCCAGGTTACACCAAAACATTGGGAGGAAATCCTTCAACAATCTTTGTGGG
AAAGAGTATCAACTCATGTGATTGAAAACATCTACCTTCCAGCTGCGCAGACCATGAATTCAGG
AACTTTTAACACCACAGTGGATATCAAGCTTAAACAGTGGACTGATAAACAACTTCCTAATAAA
GCAGTAGAGGTTGCTTGGGAGACCCTACAAGAAGAATTTTCCCGCTTTATGACAGAACCGAAAG
GGAAAGAGCATGATGACATATTTGATAAACTTAAAGAGGCTGTTAAGGAAGAAAGTATTAAACG
ACACAAGTGGAATGACTTTGCGGAGGACAGCTTGAGGGTTATTCAACACAATGCTTTGGAAGAC
CGATCCATATCTGATAAACAGCAATGGGATGCAGCTATTTATTTTATGGAAGAGGCTCTGCAGG
CTCGTCTCAAGGATACTGAAAATGCAATTGAAAACATGGTGGGTCCAGACTGGAAAAAGAGGTG
GTTATACTGGAAGAATCGGACCCAAGAACAGTGTGTTCACAATGAAACCAAGAATGAATTGGAG
AAGATGTTGAAATGTAATGAGGAGCACCCAGCTTATCTTGCAAGTGATGAAATAACCACAGTCC
GGAAGAACCTTGAATCCCGAGGAGTAGAAGTAGATCCAAGCTTGATTAAGGATACTTGGCATCA
AGTTTATAGAAGACATTTTTTAAAAACAGCTCTAAACCATTGTAACCTTTGTCGAAGAGGTTTT
TATTACTACCAAAGGCATTTTGTAGATTCTGAGTTGGAATGCAATGATGTGGTCTTGTTTTGGC
GTATACAGCGCATGCTTGCTATCACCGCAAATACTTTAAGGCAACAACTTACAAATACTGAAGT
TAGGCGATTAGAGAAAAATGTTAAAGAGGTATTGGAAGATTTTGCTGAAGATGGTGAGAAGAAG
ATTAAATTGCTTACTGGTAAACGCGTTCAACTGGCGGAAGACCTCAAGAAAGTTAGAGAAATTC
AAGAAAAACTTGATGCTTTCATTGAAGCTCTTCATCAGGAGAAATAA.

### Results in Fibroblasts from Biopsies of OPA1 Patients

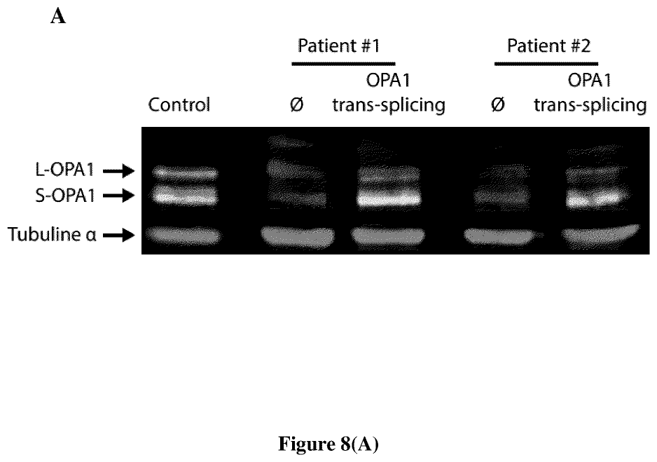

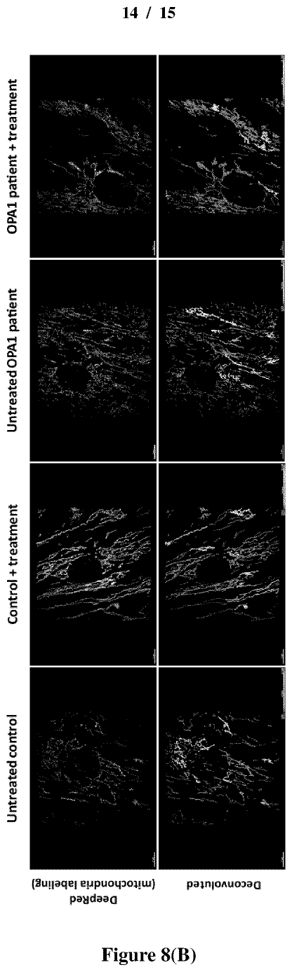

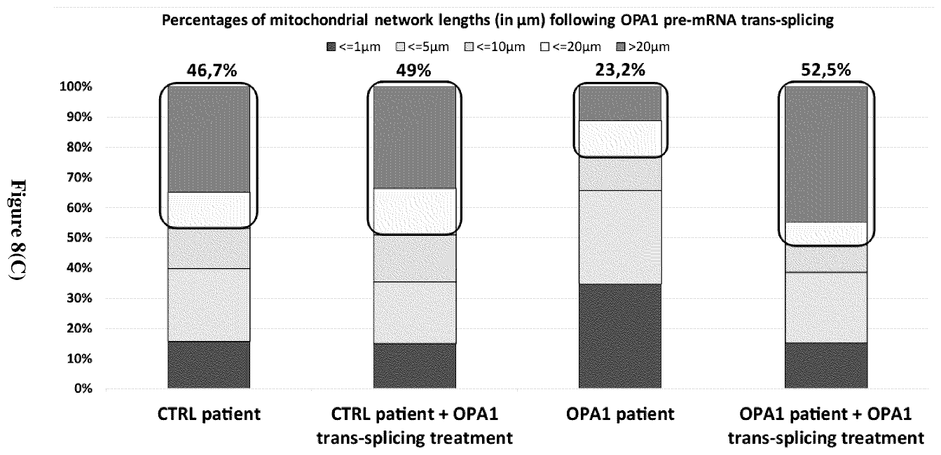

The results obtained are presented on FIG. 8.

The expression of OPA1 trans-splicing therapeutic pre-messenger RNA in fibroblasts from biopsies of OPA1 patients has established the cellular therapeutic proof of concept by: 1) Correction of OPA1 haploinsufficiency and the absence of protein overexpression (FIG. 8A); 2) induction of fusion of a pathologically fragmented mitochondrial network (FIG. 8B-C); 3) absence of deleterious effect on a healthy mitochondrial network (control) (FIG. 8B-C).

1. A nucleic acid OPA1 pre-mRNA trans-splicing molecule comprising, operatively linked in a 5′-to-3′ direction or a 3′-to-5′ direction:
(a) a binding domain configured to bind to an OPA1 target sequence comprising an intron, an intron/exon junction or an exon;
(b) a splicing domain configured to mediate trans-splicing; and
(c) a coding domain comprising a functional OPA1 exon,
wherein the nucleic acid OPA1 pre-mRNA trans-splicing molecule is configured to trans-splice the coding domain to an endogenous mutated OPA1 exon adjacent to the OPA1 target sequence, thereby replacing the endogenous mutated OPA1 exon with the functional OPA1 exon and correcting a mutation in OPA1 transcripts.

2. The nucleic acid OPA1 pre-mRNA trans-splicing molecule according to claim 1, wherein the OPA1 target sequence comprising an intron, an intron/exon junction or an exon is Homo sapiens OPA1 intron 8-9/Exon 9.

3. The nucleic acid OPA1 pre-mRNA trans-splicing molecule according to or claim 2, wherein the Homo sapiens OPA1 intron 8-9/Exon 9 consisting of SEQ ID NO: 1.

4. The nucleic acid OPA1 pre-mRNA trans-splicing molecule according to claim 3, wherein the binding domain is configured to bind the OPA1 target sequence at a binding site within or encompassing nucleotides 1 to 52 of SEQ ID NO: 1, or a binding site within or encompassing nucleotides 1 to 50 of SEQ ID NO: 1, or at a binding site within or encompassing nucleotides 261 to 315 of SEQ ID NO: 1, or at a binding site within or encompassing nucleotides 361 to 603 of SEQ ID NO: 1, or at a binding site within or encompassing nucleotides 361 to 460 of SEQ ID NO: 1, or at a binding site within or encompassing nucleotides 375 to 460 of SEQ ID NO: 1; or nucleotides 461 to 603 of SEQ ID NO: 1, or at a binding site within or encompassing nucleotides 510 to 784 of SEQ ID NO: 1, or at a binding site within or encompassing nucleotides 510 to 756 of SEQ ID NO 1, or at a binding site within or encompassing nucleotides 510 to 726 of SEQ ID NO 1, or at a binding site within or encompassing nucleotides 518 to 784 of SEQ ID NO 1, or at a binding site within or encompassing nucleotides 518 to 756 of SEQ ID NO 1, or at a binding site within or encompassing nucleotides 518 to 726 of SEQ ID NO 1, or at a binding site within or encompassing nucleotides 561 to 784 of SEQ ID NO 1, or at a binding site within or encompassing nucleotides 561 to 756 of SEQ ID NO 1, or at a binding site within or encompassing nucleotides 561 to 726 of SEQ ID NO 1, or at a binding site within or encompassing nucleotides 627 to 784 of SEQ ID NO 1, or at a binding site within or encompassing nucleotides 627 to 756 of SEQ ID NO 1, or at a binding site within or encompassing nucleotides 627 to 726 of SEQ ID NO 1, or at a combination of said binding sites.

5. The nucleic acid OPA1 pre-mRNA trans-splicing molecule according to claim 3, wherein the binding domain is complementary to six or more consecutive nucleotides of a binding site within or encompassing nucleotides 1 to 52 of SEQ ID NO: 1, or nucleotides 1 to 50 of SEQ ID NO: 1, or nucleotides 261 to 315 of SEQ ID NO: 1, or nucleotides 361 to 603 of SEQ ID NO: 1, or nucleotides 361 to 460 of SEQ ID NO: 1, or nucleotides 375 to 460 of SEQ ID NO: 1; or nucleotides 461 to 603 of SEQ ID NO: 1, or nucleotides 510 to 784 of SEQ ID NO: 1, or nucleotides 510 to 756 of SEQ ID NO 1, or nucleotides 510 to 726 of SEQ ID NO 1, or nucleotides 518 to 784 of SEQ ID NO 1, or nucleotides 518 to 756 of SEQ ID NO 1, or nucleotides 518 to 726 of SEQ ID NO 1, or nucleotides 561 to 784 of SEQ ID NO 1, or nucleotides 561 to 756 of SEQ ID NO 1, or nucleotides 561 to 726 of SEQ ID NO 1, or nucleotides 627 to 784 of SEQ ID NO 1, or nucleotides 627 to 756 of SEQ ID NO 1, or nucleotides 627 to 726 of SEQ ID NO 1, or at a combination of said binding sites.

6. The nucleic acid OPA1 pre-mRNA trans-splicing molecule according to claim 3, wherein the binding domain comprises, or consists of, SEQ ID NO: 2 (BD #114) or is a fragment thereof; or the binding domain comprises, or consists of, SEQ ID NO: 3 (BD #128) or is a fragment thereof; or the binding domain comprises, or consists of, SEQ ID NO: 4 (BD #162) or is a fragment thereof; or the binding domain comprises, or consists of, SEQ ID NO: 5 (BD #135) or is a fragment thereof; or the binding domain comprises, or consists of, SEQ ID NO: 6 (BD #106) or is a fragment thereof; or the binding domain comprises, or consists of, SEQ ID NO: 7 (BD #100) or is a fragment thereof; or the binding domain comprises, or consists of, SEQ ID NO: 8 (BD #130) or is a fragment thereof; or the binding domain comprises, or consists of, SEQ ID NO: 9 (BD #158) or is a fragment thereof; or the binding domain comprises, or consists of, SEQ ID NO: 10 (BD #166) or is a fragment thereof; or the binding domain comprises, or consists of, SEQ ID NO: 11 (BD #196) or is a fragment thereof; or the binding domain comprises, or consists of, SEQ ID NO: 12 (BD #224) or is a fragment thereof; or the binding domain comprises, or consists of, SEQ ID NO: 13 (BD #209) or is a fragment thereof; or the binding domain comprises, or consists of, SEQ ID NO: 14 (BD #239) or is a fragment thereof; or the binding domain comprises, or consists of, SEQ ID NO: 15 (BD #267) or is a fragment thereof; or the binding domain comprises, or consists of, SEQ ID NO: 16 (BD #217) or is a fragment thereof; or the binding domain comprises, or consists of, SEQ ID NO: 17 (BD #247) or is a fragment thereof; or the binding domain comprises, or consists of, SEQ ID NO: 18 (BD #275) or is a fragment thereof; or the binding domain is a combination of said binding domains.

7. The nucleic acid OPA1 pre-mRNA trans-splicing molecule according to claim 1, wherein the mutation is in any one of OPA1 exons 10 to 31.

8. The nucleic acid OPA1 pre-mRNA trans-splicing molecule according to claim 7, wherein the mutation is associated with a hereditary optic neuropathy.

9. The nucleic acid OPA1 pre-mRNA trans-splicing molecule according to claim 8, wherein the mutation is associated with a hereditary optic neuropathy selected from Autosomal Dominant Optic Atrophy (ADOA), Autosomal Dominant Optic Atrophy Plus (ADOA+) and Behr's Syndrome.

10. The nucleic acid OPA1 pre-mRNA trans-splicing molecule according to claim 7, wherein the mutation is associated with the optic neuropathy: susceptibility to normal tension glaucoma.

11. The nucleic acid OPA1 pre-mRNA trans-splicing molecule according to claim 8, wherein the mutation is associated with an extra-ophthalmic disease selected from sensorineural deafness, peripheral neuropathies, Parkinson syndrome and mitochondrial myopathy.

12. A nucleic acid OPA1 pre-mRNA trans-splicing molecule according to claim 11, wherein the mitochondrial myopathy is mitochondrial depletion syndrome 14.

13. The nucleic acid OPA1 pre-mRNA trans-splicing molecule according to claim 1, wherein the coding domain comprises functional OPA1 exons 9 to 31.

14. The nucleic acid OPA1 pre-mRNA trans-splicing molecule according to claim 1, wherein the nucleic acid OPA1 pre-mRNA trans-splicing molecule consists of SEQ ID NO: 27 (OPA1 RTMₜₕ#114), or SEQ ID NO: 28 (OPA1 RTMₜₕ#128), or SEQ ID NO: 30 (OPA1 RTMₜₕ#162), or SEQ ID NO: 29 (OPA1 RTMₜₕ#135) or SEQ ID NO: 26 (OPA1 RTMₜₕ#106), or SEQ ID NO: 31 (OPA1 RTMₜₕ#100), or SEQ ID NO: 32 (OPA1 RTMₜₕ#130), or SEQ ID NO: 33 (OPA1 RTMₜₕ#158), or SEQ ID NO: 34 (OPA1 RTMₜₕ#166), or SEQ ID NO: 35 (OPA1 RTMₜₕ#196), or SEQ ID NO: 36 (OPA1 RTMₜₕ#224), or SEQ ID NO: 37 (OPA1 RTMₜₕ#209), or SEQ ID NO: 38 (OPA1 RTMₜₕ#239), or SEQ ID NO: 39 (OPA1 RTMₜₕ#267), or SEQ ID NO: 40 (OPA1 RTMₜₕ#217), or SEQ ID NO: 41 (OPA1 RTMₜₕ#247), or SEQ ID NO: 42 (OPA1 RTMₜₕ#275).

15. A cloning vector comprising a nucleic acid OPA1 pre-mRNA trans-splicing molecule according to claim 1.

16. The cloning vector according to claim 15, wherein said cloning vector is selected from plasmids, minicircles, cosmids, YAC vectors, BAC vectors, and viral vectors.

17. The cloning vector according to claim 16, wherein the viral vector is an Adeno-Associated Virus (AAV), in particular a recombinant Adeno-Associated Virus (rAAV), a single-stranded Adeno-Associated Virus (ssAAV) or a self-complementary Adeno-Associated Virus (scAAV).

18. The cloning vector according to claim 15, wherein the recombinant adeno-associated virus targets retinal ganglion cells, photoreceptor cells, amacrine cells, horizontal cells, bipolar cells, or cells of the auditory nerve.

19. A cell comprising a nucleic acid OPA1 pre-mRNA trans-splicing molecule according to claim 1.

20-26.

28. A pharmaceutical composition comprising a nucleic acid OPA1 pre-mRNA trans-splicing molecule according to claim 1, or a cloning vector comprising said nucleic acid OPA1 pre-mRNA trans-splicing molecule, or a cell vector comprising said nucleic acid OPA1 pre-mRNA trans-splicing molecule, and at least one pharmaceutically acceptable carrier or excipient.

29. A method for treating or preventing a disease or disorder associated with a OPA1 gene mutation in a subject, the method comprising a step of administering to said subject in need thereof a therapeutically effective amount of:
a nucleic acid OPA1 pre-mRNA trans-splicing molecule according to claim 1, or a cloning vector comprising said nucleic acid OPA1 pre-mRNA trans-splicing molecule, or a cell comprising said nucleic acid OPA1 pre-mRNA trans-splicing molecule; or
cells which have been contacted with said nucleic acid OPA1 pre-mRNA trans-splicing molecule or with a cloning vector comprising said nucleic acid OPA1 pre-mRNA trans-splicing molecule.

30. The method according to claim 29, wherein the disease or disorder associated with a OPA1 gene mutation is a hereditary optic neuropathy or an extra-ophthalmic disease.

31. The method according to claim 30, wherein the disease or disorder associated with a OPA1 gene mutation is a hereditary optic neuropathy selected from Autosomal Dominant Optic Atrophy (ADOA), Autosomal Dominant Optic Atrophy Plus (ADOA+) and Behr's Syndrome.

32. The method according to claim 30, wherein the disease or disorder associated with a OPA1 gene mutation is the optic neuropathy: susceptibility to normal tension glaucoma.

33. The method according to claim 30, wherein the disease or disorder associated with a OPA1 gene mutation is associated with an extra-ophthalmic disease selected from sensorineural deafness, peripheral neuropathies, Parkinson syndrome and mitochondrial myopathy.

34. The method according to claim 33, wherein mitochondrial myopathy is mitochondrial depletion syndrome 14.


SEQUENCE LISTING (89 sequences)

SEQ ID NO: 1 - DNA, 784 bp, Homo sapiens
  source 1..784: mol_type = genomic DNA
gtatgtgaaaaattgatagtgaacttgccaattagcaaaaaaagaagcagcttagcttcctaaaaattatgtgtatatatgtacacatacacatatatacatactagatgtaggcatttatattttttatgtaatcttacatgttccaagtaatgtcttaagcaatattatttgactattttagttcattataaatattaataaatataagtacattatatttatgagttactgtatgtgttataaaggaagatatttggctttatatgtcttaatattagtaatatttaaatactgaacagtggattaaattagccatatgcgtgaaatttaagctaatagaattgaaaatgtgtttgtaaacagtaaactagctatggaaaagatttatggaaagttaataacctggttttagaaatactggtttaaattagcacaagtttttaaaataaaaattaggctataaacagtggatccagttatagttttgctgttcctattttcaatgtgcacacatgcatcaatcaccatttttttacggattttaaaatattttttcacctgtagaaattttaaaagactaaaaaactcagagcagcattacaaataggttttaattttaatttggtatcagaaaaatatgaataagtgttctttgttttgtgggaaggttgttgtggttggagatcagagtgctggaaagactagtgtgttggaaatgattgcccaagctcgaatattcccaagaggatctggggagatgatgacacgttctccagttaag

SEQ ID NO: 2 - DNA, 55 bp, synthetic construct
  source 1..55: mol_type = other DNA
ctaatttaatccactgttcagtatttaaatattactaatattaagacatataaag

SEQ ID NO: 3 - DNA, 86 bp, synthetic construct
  source 1..86: mol_type = other DNA
cctaatttttattttaaaaacttgtgctaatttaaaccagtatttctaaaaccaggttattaactttccataaatcttttccatag

SEQ ID NO: 4 - DNA, 143 bp, synthetic construct
  source 1..143: mol_type = other DNA
taatgctgctctgagttttttagtcttttaaaatttctacaggtgaaaaaatattttaaaatccgtaaaaaaatggtgattgatgcatgtgtgcacattgaaaataggaacagcaaaactataactggatccactgtttatag

SEQ ID NO: 5 - DNA, 105 bp, synthetic construct
  source 1..105: mol_type = other DNA
ctgcttctttttttgctaattggcaagttcactatcaatttttcacatacctttatatgtcttaatattagtaatatttaaatactgaacagtggattaaattag

SEQ ID NO: 6 - DNA, 250 bp, synthetic construct
  source 1..250: mol_type = other DNA
cctaatttttattttaaaaacttgtgctaatttaaaccagtatttctaaaaccaggttattaactttccataaatcttttccatagctgcttctttttttgctaattggcaagttcactatcaatttttcacatacctttatatgtcttaatattagtaatatttaaatactgaacagtggattaaattagcttaaatttcacgcatatggctagtttactgtttacaaacacattttcaattctattag

SEQ ID NO: 7 - DNA, 100 bp, synthetic construct
  source 1..100: mol_type = other DNA
gcaatcatttccaacacactagtctttccagcactctgatctccaaccacaacaaccttcccacaaaacaaagaacacttattcatatttttctgatacc

SEQ ID NO: 8 - DNA, 130 bp, synthetic construct
  source 1..130: mol_type = other DNA
ccagatcctcttgggaatattcgagcttgggcaatcatttccaacacactagtctttccagcactctgatctccaaccacaacaaccttcccacaaaacaaagaacacttattcatatttttctgatacc

SEQ ID NO: 9 - DNA, 158 bp, synthetic construct
  source 1..158: mol_type = other DNA
cttaactggagaacgtgtcatcatctccccagatcctcttgggaatattcgagcttgggcaatcatttccaacacactagtctttccagcactctgatctccaaccacaacaaccttcccacaaaacaaagaacacttattcatatttttctgatacc

SEQ ID NO: 10 - DNA, 166 bp, synthetic construct
  source 1..166: mol_type = other DNA
gcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacagg

SEQ ID NO: 11 - DNA, 196 bp, synthetic construct
  source 1..196: mol_type = other DNA
ccagatcctcttgggaatattcgagcttgggcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacagg

SEQ ID NO: 12 - DNA, 224 bp, synthetic construct
  source 1..224: mol_type = other DNA
cttaactggagaacgtgtcatcatctccccagatcctcttgggaatattcgagcttgggcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacagg

SEQ ID NO: 13 - DNA, 209 bp, synthetic construct
  source 1..209: mol_type = other DNA
gcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggtgaaaaaatattttaaaatccgtaaaaaaatggtgattgatgc

SEQ ID NO: 14 - DNA, 239 bp, synthetic construct
  source 1..239: mol_type = other DNA
ccagatcctcttgggaatattcgagcttgggcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggtgaaaaaatattttaaaatccgtaaaaaaatggtgattgatgc

SEQ ID NO: 15 - DNA, 267 bp, synthetic construct
  source 1..267: mol_type = other DNA
cttaactggagaacgtgtcatcatctccccagatcctcttgggaatattcgagcttgggcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggtgaaaaaatattttaaaatccgtaaaaaaatggtgattgatgc

SEQ ID NO: 16 - DNA, 217 bp, synthetic construct
  source 1..217: mol_type = other DNA
gcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggtgaaaaaatattttaaaatccgtaaaaaaatggtgattgatgcatgtgtgc

SEQ ID NO: 17 - DNA, 247 bp, synthetic construct
  source 1..247: mol_type = other DNA
ccagatcctcttgggaatattcgagcttgggcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggtgaaaaaatattttaaaatccgtaaaaaaatggtgattgatgcatgtgtgc

SEQ ID NO: 18 - DNA, 275 bp, synthetic construct
  source 1..275: mol_type = other DNA
cttaactggagaacgtgtcatcatctccccagatcctcttgggaatattcgagcttgggcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggtgaaaaaatattttaaaatccgtaaaaaaatggtgattgatgcatgtgtgc

SEQ ID NO: 19 - skipped

SEQ ID NO: 20 - DNA, 16 bp, synthetic construct
  source 1..16: mol_type = other DNA
tcttcttttttttctg

SEQ ID NO: 21 - skipped

SEQ ID NO: 22 - DNA, 27 bp, synthetic construct
  source 1..27: mol_type = other DNA
gagaacattattatagcgttgctcgag

SEQ ID NO: 23 - DNA, 60 bp, synthetic construct
  source 1..60: mol_type = other DNA
gagaacattattatagcgttgctcgagtactaactggtacctcttcttttttttctgcag

SEQ ID NO: 24 - DNA, 2013 bp, Homo sapiens
  source 1..2013: mol_type = other DNA
gtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 25 - DNA, 2040 bp, synthetic construct
  source 1..2040: mol_type = other DNA
ctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataagtcgacggtaccgcgggcccg

SEQ ID NO: 26 - DNA, 2326 bp, synthetic construct
  source 1..2326: mol_type = other DNA
cctaatttttattttaaaaacttgtgctaatttaaaccagtatttctaaaaccaggttattaactttccataaatcttttccatagctgcttctttttttgctaattggcaagttcactatcaatttttcacatacctttatatgtcttaatattagtaatatttaaatactgaacagtggattaaattagcttaaatttcacgcatatggctagtttactgtttacaaacacattttcaattctattagaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 27 - DNA, 2131 bp, synthetic construct
  source 1..2131: mol_type = other DNA
ctaatttaatccactgttcagtatttaaatattactaatattaagacatataaagaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 28 - DNA, 2162 bp, synthetic construct
  source 1..2162: mol_type = other DNA
cctaatttttattttaaaaacttgtgctaatttaaaccagtatttctaaaaccaggttattaactttccataaatcttttccatagaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 29 - DNA, 2181 bp, synthetic construct
  source 1..2181: mol_type = other DNA
ctgcttctttttttgctaattggcaagttcactatcaatttttcacatacctttatatgtcttaatattagtaatatttaaatactgaacagtggattaaattagaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 30 - DNA, 2219 bp, synthetic construct
  source 1..2219: mol_type = other DNA
taatgctgctctgagttttttagtcttttaaaatttctacaggtgaaaaaatattttaaaatccgtaaaaaaatggtgattgatgcatgtgtgcacattgaaaataggaacagcaaaactataactggatccactgtttatagaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 31 - DNA, 2185 bp, synthetic construct
  source 1..2185: mol_type = other DNA
gcaatcatttccaacacactagtctttccagcactctgatctccaaccacaacaaccttcccacaaaacaaagaacacttattcatatttttctgataccatcgatgttaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 32 - DNA, 2215 bp, synthetic construct
  source 1..2215: mol_type = other DNA
ccagatcctcttgggaatattcgagcttgggcaatcatttccaacacactagtctttccagcactctgatctccaaccacaacaaccttcccacaaaacaaagaacacttattcatatttttctgataccatcgatgttaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 33 - DNA, 2243 bp, synthetic construct
  source 1..2243: mol_type = other DNA
cttaactggagaacgtgtcatcatctccccagatcctcttgggaatattcgagcttgggcaatcatttccaacacactagtctttccagcactctgatctccaaccacaacaaccttcccacaaaacaaagaacacttattcatatttttctgataccatcgatgttaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 34 - DNA, 2251 bp, synthetic construct
  source 1..2251: mol_type = other DNA
gcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggatcgatgttaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 35 - DNA, 2281 bp, synthetic construct
  source 1..2281: mol_type = other DNA
ccagatcctcttgggaatattcgagcttgggcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggatcgatgttaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 36 - DNA, 2309 bp, synthetic construct
  source 1..2309: mol_type = other DNA
cttaactggagaacgtgtcatcatctccccagatcctcttgggaatattcgagcttgggcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggatcgatgttaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 37 - DNA, 2294 bp, synthetic construct
  source 1..2294: mol_type = other DNA
gcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggtgaaaaaatattttaaaatccgtaaaaaaatggtgattgatgcatcgatgttaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 38 - DNA, 2324 bp, synthetic construct
  source 1..2324: mol_type = other DNA
ccagatcctcttgggaatattcgagcttgggcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggtgaaaaaatattttaaaatccgtaaaaaaatggtgattgatgcatcgatgttaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 39 - DNA, 2352 bp, synthetic construct
  source 1..2352: mol_type = other DNA
cttaactggagaacgtgtcatcatctccccagatcctcttgggaatattcgagcttgggcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggtgaaaaaatattttaaaatccgtaaaaaaatggtgattgatgcatcgatgttaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 40 - DNA, 2302 bp, synthetic construct
  source 1..2302: mol_type = other DNA
gcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggtgaaaaaatattttaaaatccgtaaaaaaatggtgattgatgcatgtgtgcatcgatgttaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 41 - DNA, 2332 bp, synthetic construct
  source 1..2332: mol_type = other DNA
ccagatcctcttgggaatattcgagcttgggcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggtgaaaaaatattttaaaatccgtaaaaaaatggtgattgatgcatgtgtgcatcgatgttaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 42 - DNA, 2360 bp, synthetic construct
  source 1..2360: mol_type = other DNA
cttaactggagaacgtgtcatcatctccccagatcctcttgggaatattcgagcttgggcaatcatttccaacacactagtctttccagcactctgatctccaaccacaacaaccttcccacaaaacaaagaacacttattcatatttttctgataccaaattaaaattaaaacctatttgtaatgctgctctgagttttttagtcttttaaaatttctacaggtgaaaaaatattttaaaatccgtaaaaaaatggtgattgatgcatgtgtgcatcgatgttaacgagaacattattatagcgttgctcgagtactaactggtacctcttcttttttttctgcaggtagtagtcgtaggtgatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 43 - DNA, 29 bp, synthetic construct
  source 1..29: mol_type = other DNA
gtatgtgaaaaattgatagtgaacttgcc

SEQ ID NO: 44 - DNA, 25 bp, synthetic construct
  source 1..25: mol_type = other DNA
cttaactggagaacgtgtcatcatc

SEQ ID NO: 45 - DNA, 45 bp, synthetic construct
  source 1..45: mol_type = other DNA
gccgaggtaagagatatcgtatgtgaaaaattgatagtgaacttg

SEQ ID NO: 46 - DNA, 39 bp, synthetic construct
  source 1..39: mol_type = other DNA
tagactcgagcggccgccttaactggagaacgtgtcatc

SEQ ID NO: 47 - DNA, 23 bp, synthetic construct
  source 1..23: mol_type = other DNA
atcttcttcgaggatgacggcaa

SEQ ID NO: 48 - DNA, 36 bp, synthetic construct
  source 1..36: mol_type = other DNA
gctatggaaaagatttatggaaagttaataacctgg

SEQ ID NO: 49 - DNA, 30 bp, synthetic construct
  source 1..30: mol_type = other DNA
gtttaaactcaatggtgatggtgatgatga

SEQ ID NO: 50 - DNA, 33 bp, synthetic construct
  source 1..33: mol_type = other DNA
aaactataactggatccactgtttatagcctaa

SEQ ID NO: 51 - DNA, 57 bp, synthetic construct
  source 1..57: mol_type = other DNA
ttctcgttaacatcgatggtatcagaaaaatatgaataagtgttctttgttttgtgg

SEQ ID NO: 52 - DNA, 53 bp, synthetic construct
  source 1..53: mol_type = other DNA
ttctcgttaacatcgatcctgtagaaattttaaaagactaaaaaactcagagc

SEQ ID NO: 53 - DNA, 62 bp, synthetic construct
  source 1..62: mol_type = other DNA
ttctcgttaacatcgatgcatcaatcaccatttttttacggattttaaaatattttttcacc

SEQ ID NO: 54 - DNA, 48 bp, synthetic construct
  source 1..48: mol_type = other DNA
ttctcgttaacatcgatgcacacatgcatcaatcaccatttttttacg

SEQ ID NO: 55 - DNA, 45 bp, synthetic construct
  source 1..45: mol_type = other DNA
acccttcaccgaattcgcaatcatttccaacacactagtctttcc

SEQ ID NO: 56 - DNA, 42 bp, synthetic construct
  source 1..42: mol_type = other DNA
acccttcaccgaattcccagatcctcttgggaatattcgagc

SEQ ID NO: 57 - DNA, 44 bp, synthetic construct
  source 1..44: mol_type = other DNA
acccttcaccgaattccttaactggagaacgtgtcatcatctcc

SEQ ID NO: 58 - DNA, 22 bp, synthetic construct
  source 1..22: mol_type = other DNA
ggctaactagagaacccactgc

SEQ ID NO: 59 - DNA, 22 bp, synthetic construct
  source 1..22: mol_type = other DNA
ccatcctccttgaaatcggtgc

SEQ ID NO: 60 - DNA, 195 bp, synthetic construct
  source 1..195: mol_type = other DNA
ctatggaaaagatttatggaaagttaataacctggttttagaaatactggtttaaattagcacaagtttttaaaataaaaattagggtatgtgaaaaattgatagtgaacttgccaattagcaaaaaaagaagcagccatatgcgtgaaatttaagctaatagaattgaaaatgtgtttgtaaacagtaaactag

SEQ ID NO: 61 - DNA, 55 bp, synthetic construct
  source 1..55: mol_type = other DNA
ctttatatgtcttaatattagtaatatttaaatactgaacagtggattaaattag

SEQ ID NO: 62 - DNA, 86 bp, synthetic construct
  source 1..86: mol_type = other DNA
ctatggaaaagatttatggaaagttaataacctggttttagaaatactggtttaaattagcacaagtttttaaaataaaaattagg

SEQ ID NO: 63 - DNA, 50 bp, synthetic construct
  source 1..50: mol_type = other DNA
gtatgtgaaaaattgatagtgaacttgccaattagcaaaaaaagaagcag

SEQ ID NO: 64 - DNA, 143 bp, synthetic construct
  source 1..143: mol_type = other DNA
ctataaacagtggatccagttatagttttgctgttcctattttcaatgtgcacacatgcatcaatcaccatttttttacggattttaaaatattttttcacctgtagaaattttaaaagactaaaaaactcagagcagcatta

SEQ ID NO: 65 - DNA, 47 bp, synthetic construct
  source 1..47: mol_type = other DNA
cgagctcaagcttcgaattccctaatttttattttaaaaacttgtgc

SEQ ID NO: 66 - DNA, 46 bp, synthetic construct
  source 1..46: mol_type = other DNA
cgagctcaagcttcgaattcctaatttaatccactgttcagtattt

SEQ ID NO: 67 - DNA, 42 bp, synthetic construct
  source 1..42: mol_type = other DNA
cgagctcaagcttcgaattcctgcttctttttttgctaattg

SEQ ID NO: 68 - DNA, 43 bp, synthetic construct
  source 1..43: mol_type = other DNA
cgagctcaagcttcgaattctaatgctgctctgagttttttag

SEQ ID NO: 69 - DNA, 39 bp, synthetic construct
  source 1..39: mol_type = other DNA
ctgcaggtagtagtcgtaggtgatcagagtgctggaaag

SEQ ID NO: 70 - DNA, 42 bp, synthetic construct
  source 1..42: mol_type = other DNA
ctgatcacctacgactactacctgcagaaaaaaaagaagagg

SEQ ID NO: 71 - DNA, 45 bp, synthetic construct
  source 1..45: mol_type = other DNA
cgggcccgcggtaccgtcgacttatttctcctgatgaagagcttc

SEQ ID NO: 72 - DNA, 354 bp, synthetic construct
  source 1..354: mol_type = other DNA
cgagctcaagcttcgaattccctaatttttattttaaaaacttgtgctaatttaaaccagtatttctaaaaccaggttattaactttccataaatcttttccatagctgcttctttttttgctaattggcaagttcactatcaatttttcacatacctttatatgtcttaatattagtaatatttaaatactgaacagtggattaaattagcttaaatttcacgcatatggctagtttactgtttacaaacacattttcaattctattagaacgagaacattattatagcgttgctcgagtactaactggtacctcttcttttttttctgcaggtagtagtcgtaggtgatcag

SEQ ID NO: 73 - DNA, 159 bp, synthetic construct
  source 1..159: mol_type = other DNA
cgagctcaagcttcgaattcctaatttaatccactgttcagtatttaaatattactaatattaagacatataaagaacgagaacattattatagcgttgctcgagtactaactggtacctcttcttttttttctgcaggtagtagtcgtaggtgatcag

SEQ ID NO: 74 - DNA, 190 bp, synthetic construct
  source 1..190: mol_type = other DNA
cgagctcaagcttcgaattccctaatttttattttaaaaacttgtgctaatttaaaccagtatttctaaaaccaggttattaactttccataaatcttttccatagaacgagaacattattatagcgttgctcgagtactaactggtacctcttcttttttttctgcaggtagtagtcgtaggtgatcag

SEQ ID NO: 75 - DNA, 209 bp, synthetic construct
  source 1..209: mol_type = other DNA
cgagctcaagcttcgaattcctgcttctttttttgctaattggcaagttcactatcaatttttcacatacctttatatgtcttaatattagtaatatttaaatactgaacagtggattaaattagaacgagaacattattatagcgttgctcgagtactaactggtacctcttcttttttttctgcaggtagtagtcgtaggtgatcag

SEQ ID NO: 76 - DNA, 247 bp, synthetic construct
  source 1..247: mol_type = other DNA
cgagctcaagcttcgaattctaatgctgctctgagttttttagtcttttaaaatttctacaggtgaaaaaatattttaaaatccgtaaaaaaatggtgattgatgcatgtgtgcacattgaaaataggaacagcaaaactataactggatccactgtttatagaacgagaacattattatagcgttgctcgagtactaactggtacctcttcttttttttctgcaggtagtagtcgtaggtgatcag

SEQ ID NO: 77 - DNA, 100 bp, synthetic construct
  source 1..100: mol_type = other DNA
ggtatcagaaaaatatgaataagtgttctttgttttgtgggaaggttgttgtggttggagatcagagtgctggaaagactagtgtgttggaaatgattgc

SEQ ID NO: 78 - DNA, 130 bp, synthetic construct
  source 1..130: mol_type = other DNA
ggtatcagaaaaatatgaataagtgttctttgttttgtgggaaggttgttgtggttggagatcagagtgctggaaagactagtgtgttggaaatgattgcccaagctcgaatattcccaagaggatctgg

SEQ ID NO: 79 - DNA, 158 bp, synthetic construct
  source 1..158: mol_type = other DNA
ggtatcagaaaaatatgaataagtgttctttgttttgtgggaaggttgttgtggttggagatcagagtgctggaaagactagtgtgttggaaatgattgcccaagctcgaatattcccaagaggatctggggagatgatgacacgttctccagttaag

SEQ ID NO: 80 - DNA, 166 bp, synthetic construct
  source 1..166: mol_type = other DNA
cctgtagaaattttaaaagactaaaaaactcagagcagcattacaaataggttttaattttaatttggtatcagaaaaatatgaataagtgttctttgttttgtgggaaggttgttgtggttggagatcagagtgctggaaagactagtgtgttggaaatgattgc

SEQ ID NO: 81 - DNA, 196 bp, synthetic construct
  source 1..196: mol_type = other DNA
cctgtagaaattttaaaagactaaaaaactcagagcagcattacaaataggttttaattttaatttggtatcagaaaaatatgaataagtgttctttgttttgtgggaaggttgttgtggttggagatcagagtgctggaaagactagtgtgttggaaatgattgcccaagctcgaatattcccaagaggatctgg

SEQ ID NO: 82 - DNA, 224 bp, synthetic construct
  source 1..224: mol_type = other DNA
cctgtagaaattttaaaagactaaaaaactcagagcagcattacaaataggttttaattttaatttggtatcagaaaaatatgaataagtgttctttgttttgtgggaaggttgttgtggttggagatcagagtgctggaaagactagtgtgttggaaatgattgcccaagctcgaatattcccaagaggatctggggagatgatgacacgttctccagttaag

SEQ ID NO: 83 - DNA, 209 bp, synthetic construct
  source 1..209: mol_type = other DNA
gcatcaatcaccatttttttacggattttaaaatattttttcacctgtagaaattttaaaagactaaaaaactcagagcagcattacaaataggttttaattttaatttggtatcagaaaaatatgaataagtgttctttgttttgtgggaaggttgttgtggttggagatcagagtgctggaaagactagtgtgttggaaatgattgc

SEQ ID NO: 84 - DNA, 239 bp, synthetic construct
  source 1..239: mol_type = other DNA
gcatcaatcaccatttttttacggattttaaaatattttttcacctgtagaaattttaaaagactaaaaaactcagagcagcattacaaataggttttaattttaatttggtatcagaaaaatatgaataagtgttctttgttttgtgggaaggttgttgtggttggagatcagagtgctggaaagactagtgtgttggaaatgattgcccaagctcgaatattcccaagaggatctgg

SEQ ID NO: 85 - DNA, 267 bp, synthetic construct
  source 1..267: mol_type = other DNA
gcatcaatcaccatttttttacggattttaaaatattttttcacctgtagaaattttaaaagactaaaaaactcagagcagcattacaaataggttttaattttaatttggtatcagaaaaatatgaataagtgttctttgttttgtgggaaggttgttgtggttggagatcagagtgctggaaagactagtgtgttggaaatgattgcccaagctcgaatattcccaagaggatctggggagatgatgacacgttctccagttaag

SEQ ID NO: 86 - DNA, 217 bp, synthetic construct
  source 1..217: mol_type = other DNA
gcacacatgcatcaatcaccatttttttacggattttaaaatattttttcacctgtagaaattttaaaagactaaaaaactcagagcagcattacaaataggttttaattttaatttggtatcagaaaaatatgaataagtgttctttgttttgtgggaaggttgttgtggttggagatcagagtgctggaaagactagtgtgttggaaatgattgc

SEQ ID NO: 87 - DNA, 247 bp, synthetic construct
  source 1..247: mol_type = other DNA
gcacacatgcatcaatcaccatttttttacggattttaaaatattttttcacctgtagaaattttaaaagactaaaaaactcagagcagcattacaaataggttttaattttaatttggtatcagaaaaatatgaataagtgttctttgttttgtgggaaggttgttgtggttggagatcagagtgctggaaagactagtgtgttggaaatgattgcccaagctcgaatattcccaagaggatctgg

SEQ ID NO: 88 - DNA, 275 bp, synthetic construct
  source 1..275: mol_type = other DNA
gcacacatgcatcaatcaccatttttttacggattttaaaatattttttcacctgtagaaattttaaaagactaaaaaactcagagcagcattacaaataggttttaattttaatttggtatcagaaaaatatgaataagtgttctttgttttgtgggaaggttgttgtggttggagatcagagtgctggaaagactagtgtgttggaaatgattgcccaagctcgaatattcccaagaggatctggggagatgatgacacgttctccagttaag

SEQ ID NO: 89 - DNA, 78 bp, synthetic construct
  source 1..78: mol_type = other DNA
gaattcatcgatgttaacgagaacattattatagcgttgctcgagtactaactggtacctcttcttttttttctgcag